(19) United States
(12) Patent Application Publication
(10) Pub. No.: US 2010/0075973 A1
(43) Pub. Date: Mar. 25, 2010

(54) POLO-LIKE KINASE INHIBITORS

(75) Inventors: Sheldon X. Cao, San Diego, CA (US); Takashi Ichikawa, Tokyo (JP); Andre A. Kiryanov, San Diego, CA (US); Christopher McBride, San Diego, CA (US); Srinivasa Reddy Natala, San Diego, CA (US); Stephen W. Kaldor, Del Mar, CA (US); Jeffrey A. Stafford, San Diego, CA (US)

Correspondence Address: TAKEDA SAN DIEGO, INC., 10410 SCIENCE CENTER DRIVE, SAN DIEGO, CA, 92121, US

(73) Assignee: TAKEDA PHARMACEUTICAL COMPANY LIMITED, Osaka (JP)

(21) Appl. No.: 12/544,419

(22) Filed: Aug. 20, 2009

Related U.S. Application Data
(60) Provisional application No. 61/092,704, filed on Aug. 28, 2008.
(60) Provisional application No. 61/171,371, filed on Apr. 21, 2009.

(51) Int. Cl. A61K 31/5025; C07D 471/14; A61P 35/00

(52) U.S. Cl. 514/250; 544/251

(57) ABSTRACT

The present invention provides PLK inhibitors of the formula

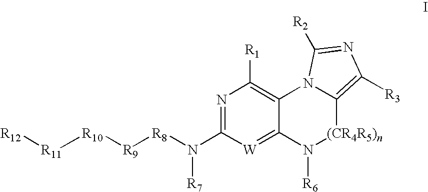

wherein the variables are as defined herein. Also provided are pharmaceutical compositions, kits and articles of manufacture comprising such compounds; methods and intermediates useful for making the compounds; and methods of using the compounds.

### RELATED APPLICATION

This application claims the benefit of U.S. Provisional Application No. 61/092,704, filed Aug. 28, 2008, and U.S. Provisional Application No. 61/171,371, filed Apr. 21, 2009; both of which are incorporated herein by reference.

### FIELD OF THE INVENTION

The present invention relates to medicinal chemistry and pharmaceutical science.

### BACKGROUND OF THE INVENTION

Kinases are responsible for the control of a wide variety of signal transduction processes by phosphorylation of their target proteins. Kinases regulated processes include proliferation, growth, differentiation, metabolism, cell cycle events, apoptosis, motility, transcription, and translation. Kinases can function, either directly or indirectly, to activate, inactivate, or modulate the activity of the target protein. These target proteins may include, for example, metabolic enzymes, regulatory proteins, receptors, cytoskeletal proteins, ion channels or pumps, or transcription factors.

Polo-like kinases (PLKs including PLK1, PLK2, PLK3 and PLK4) are serine/threonine protein kinases that are involved in the regulation of the cell cycle. In mammalian cells, PLK1 levels increase during mitosis. Target proteins for PLKs include cyclin B, a cohesin subunit of the mitotic spindle, subunits of the anaphase promoting complex, and mammalian kinesin-like protein 1 and other motor proteins. PLK1 has a role in the regulation of CDKs through phosphorylation and activation of Cdc25C leading to activation of CDK1 which is involved in cell division processes.

Modulation of kinases, such as PLK, is an especially attractive target for therapeutics. Certain inhibitors of PLK are disclosed in WO 2006/018185, WO 2007/095188, and WO 2008/076392. There is a continued need to find therapeutic agents to treat human diseases, including the treatment of multi-drug resistant cancers. The present invention provides inhibitors of PLKs.

### SUMMARY OF THE INVENTION

The present invention provides compounds of formula I:

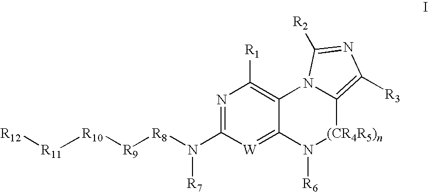

wherein

R₁ is selected from the group consisting of hydrogen, halo, amino, alkylamino, C₁₋₄ alkoxy, C₁₋₄ alkyl, and SOₓX₁

x is selected from the group consisting of 0, 1, and 2;

X₁ is selected from the group consisting of optionally substituted C₁₋₄ alkyl, optionally substituted C₃₋₈ cycloalkyl, and optionally substituted C₄₋₁₂ aryl;

R₂ is selected from the group consisting of hydrogen, halo, and C₁₋₄ alkyl;

R₃ is selected from the group consisting of amino, hydrogen, halo, nitro, cyano, optionally substituted C₁₋₆ alkyl, C₁₋₄ alkoxy, C₁₋₉ amide, C₁₋₅ oxycarbonyl, and N(X₂)(X₃);

X₂ is selected from the group consisting of hydrogen and C₁₋₄ alkyl and X₃ is selected from the group consisting of C₁₋₄ alkyl, C₁₋₇ alkylcarbonyl, and C₁₋₆ sulfonyl;

n is selected from the group consisting of 1 and 2;

R₄, each time taken, is independently selected from the group consisting of hydrogen, halo, hydroxy, optionally substituted C₁₋₁₀ alkyl, optionally substituted C₄₋₁₂ aryloxy, optionally substituted heteroC₁₋₁₀ aryloxy, optionally substituted C₃₋₈ cycloalkyl, optionally substituted C₄₋₁₂ aryl, and optionally substituted heteroC₁₋₁₀ aryl;

R₅, each time taken, is independently selected from the group consisting of hydrogen, halo, optionally substituted C₁₋₁₀ alkyl, optionally substituted C₃₋₈ cycloalkyl, optionally substituted C₄₋₁₂ aryl, and optionally substituted heteroC₁₋₁₀ aryl; or

R₄ and R₅ taken together with the carbon to which they are attached to form C═O; or

R₄ and R₅ taken together with the carbon to which they are attached form an optionally substituted C₃₋₈ cycloalkyl ring; or

when n is 2, one of R₄ or R₅ on different carbons is taken together along with the carbons to which they are attached to form an optionally substituted C₃₋₈ cycloalkyl ring;

R₆ is selected from the group consisting of hydrogen, optionally substituted C₁₋₆ alkyl, C₁₋₃ sulfonyl, and optionally substituted C₃₋₈ cycloalkyl;

R₇ is hydrogen or a substituent convertible in vivo to hydrogen;

R₈ is selected from the group consisting of optionally substituted C₄₋₁₂ arylene, optionally substituted heteroC₁₋₁₀ arylene, optionally substituted C₃₋₈ cycloalkylene, optionally substituted heteroC₃₋₆ cycloalkylene, optionally substituted C₇₋₁₂ bicycloalkylene, and optionally substituted heteroC₃₋₁₂ bicycloalkylene;

R₉ is selected from the group consisting of —CONJ₉-, —NJ₉CO—, —NJ₉-, —SO₂NJ₉-, —NJ₉SO₂—,

J₉ is selected from the group consisting of hydrogen and C₁₋₄ alkyl;

R₁₀ is selected from the group consisting of optionally substituted C₄₋₁₂ arylene, optionally substituted heteroC₁₋₁₀ arylene, optionally substituted C₃₋₈ cycloalkylene, optionally substituted heteroC₃₋₆ cycloalkylene, optionally substituted C₇₋₁₂ bicycloalkylene, and optionally substituted heteroC₃₋₁₂ bicycloalkylene;

R₁₁ is selected from the group consisting of optionally substituted C₁₋₄ alkylene, optionally substituted C₁₋₄ azaalkylene, optionally substituted C₃₋₈ cycloalkylene, and optionally substituted heteroC₃₋₆ cycloalkylene;

R₁₂ is optionally substituted C₃₋₈ cycloalkyl, when R₁₁ is selected from the group consisting of optionally substituted C₁₋₄ alkylene and optionally substituted C₁₋₄ azaalkylene;

R₁₂ is optionally substituted C₁₋₄ alkyl, when R₁₁ is selected from the group consisting of optionally substituted C₃₋₈ cycloalkylene and optionally substituted heteroC₃₋₆ cycloalkylene;

W is N or CR₁₃;

R₁₃ is selected from the group consisting of hydrogen, halo, nitro, cyano, optionally substituted C₁₋₆ alkyl, and optionally substituted C₁₋₄ alkoxy;

and pharmaceutically acceptable salts thereof.

The present invention also provides pharmaceutical compositions, comprising: a compound of formula I and a pharmaceutically acceptable excipient.

The compounds of the invention are inhibitors of PLK they are useful for the treatment of conditions associated with PLK, including cancer; inflammatory conditions; autoimmune diseases; cardiovascular diseases; infectious diseases; nephrological diseases; neurodegenerative diseases; skin disease; bone diseases; the protection of proliferating cells; and other conditions. Thus, the invention provides methods of treating conditions associated with PLK, comprising: administering to a patient in need thereof an effective amount of a compound of formula I. Further, the present invention provides for the use of compounds of formula I for the manufacture of a medicament for the treatment of conditions associated with PLK.

The present invention also provides an article of manufacture: comprising at least one compound of formula I and a label. Also provided are kits comprising at least one compound of formula I, a label, and apparatus for administration of the inhibitor.

The present invention also provides processes from making PLK inhibitors and intermediates thereof.

### DETAILED DESCRIPTION OF THE INVENTION

The term “C₁₋₇ alkylcarbonyl” refers to a carbonyl linked to a C₁₋₆ alkyl group or an optionally substituted phenyl. It is understood that the designation of C₁₋₇ for this group does not include carbon atoms or any optional substituent.

The term “C₂₋₄ alkenyl” a straight or branched alkenyl chain having from two to four carbon atoms and one or more carbon-carbon double bonds, and includes ethylene, propylene, iso-propylene, butylene, iso-butylene, sec-butylene, and the like.

The term “C₁₋₄ alkyl” refers to a straight or branched alkyl chain having from one to four carbon atoms.

The term “C₁₋₆ alkyl” refers to a straight or branched alkyl chain having from one to four carbon atoms.

The term “C₁₋₁₀ alkyl” refers to a straight or branched alkyl chain having from one to ten carbon atoms.

The term “optionally substituted C₁₋₄ alkyl” refers to a C₁₋₄ alkyl optionally having from 1 to 6 substituents independently selected from the group consisting of amino, C₂₋₄ alkenyl, C₁₋₄ alkoxy, C₁₋₉ amide, aminoC₁₋₈ alkyl, C₁₋₅ oxycarbonyl, cyano, C₃₋₈ cycloalkyl, C₃₋₈ cycloalkoxy, halo, hydroxy, nitro, oxo, and optionally substituted phenyl.

The term “optionally substituted C₁₋₆ alkyl” refers to a C₁₋₆ alkyl optionally having from 1 to 6 substituents independently selected from the group consisting of C₂₋₄ alkenyl, C₁₋₄ alkoxy, C₁₋₉ amide, C₁₋₅ oxycarbonyl, cyano, C₃₋₈ cycloalkyl, halo, hydroxy, and optionally substituted phenyl.

The term “optionally substituted C₁₋₁₀ alkyl” refers to a C₁₋₁₀ alkyl optionally having from 1 to 8 substituents independently selected from the group consisting of C₂₋₄ alkenyl, C₁₋₄ alkoxy, optionally substituted amide, amino, optionally substituted ester, cyano, C₃₋₈ cycloalkyl, C₃₋₈ cycloalkoxy, halo, hydroxy, nitro, oxo, and optionally substituted phenyl.

The term “optionally substituted C₁₋₄ alkylene” refers to a bivalent C₁₋₄ alkyl, for example one that is attached to both R₁₀ and R₁₂ of formula I, optionally having 1 to 6 substituents independently selected from the group consisting of C₂₋₄ alkenyl, C₁₋₄ alkoxy, C₁₋₉ amide, aminoC₁₋₈ alkyl, C₁₋₅ oxycarbonyl, cyano, C₃₋₈ cycloalkyl, C₃₋₈ cycloalkoxy, halo, hydroxy, nitro, oxo, and optionally substituted phenyl.

The term “C₁₋₄ alkoxy” refers to a C₁₋₄ alkyl attached through an oxygen atom.

The term “optionally substituted C₁₋₄ alkoxy” refers to a C₁₋₄ alkoxy optionally having from 1 to 5 substituents independently selected from the group consisting of C₂₋₄ alkenyl, cyano, C₃₋₈ cycloalkyl, C₃₋₈ cycloalkoxy, halo, hydroxy, nitro, oxo, and optionally substituted phenyl.

The term “C₁₋₉ amide” refers to a primary amide or one having one or two C₁₋₄ alkyls, for example amide —CONH₂ and —CONHCH₃.

The term “aminoC₁₋₈ alkyl” refers to an amino substituted with one or two C₁₋₄ alkyls, for example —NHCH₃.

The term “C₄₋₁₂ aryl” refers to a monoyclic and polycyclic unsaturated, conjugated hydrocarbon having aromatic character and having four to ten carbon atoms, and includes phenyl.

The term “optionally substituted C₄₋₁₂ aryl” refers to a C₄₋₁₂ aryl optionally having 1 to 5 substituents independently selected from the group consisting of C₁₋₄ alkyl, C₁₋₄ alkoxy, cyano, halogen, hydroxyl, nitro, and trifluoromethyl.

The term “optionally substituted C₄₋₁₂ arylene” refers to a bivalent C₄₋₁₂ aryl, for example one that is attached to both R₉ and R₁₁ of formula I, optionally having 1 to 4 substituents independently selected from the group consisting of C₁₋₄ alkyl, C₁₋₄ alkoxy, cyano, halogen, hydroxyl, nitro, and trifluoromethyl.

The term “optionally substituted C₄₋₁₂ aryloxy” refers to a C₄₋₁₂ aryl attached through an oxygen atom and optionally having 1 to 5 substituents independently selected from the group consisting of C₁₋₄ alkyl, C₁₋₄ alkoxy, cyano, halogen, hydroxyl, nitro, and trifluoromethyl.

The term “optionally substituted C₁₋₄ azaalkylene” refers to bivalent C₁₋₄ alkyl group in which a carbon atom has been replaced by a nitrogen which itself is substituted with a hydrogen or a C₁₋₄ alkyl and in which the remaining carbon atoms optionally having from 1 to 6 substituents independently selected from the group consisting of C₂₋₄ alkenyl, optionally substituted amide, optionally substituted ester, C₃₋₈ cycloalkyl, nitro, and optionally substituted phenyl.

The term “C₇₋₁₂ bicycloalkyl” refers to a bicyclic alkyl rings having from seven to twelve carbon atoms, and includes bicycl[2.3.0]heptyl and bicycle[2.2.1]heptyl.

The term “optionally substituted C₇₋₁₂ bicycloalkyl” refers to a C₇₋₁₂ bicycloalkyl optionally having from 1 to 3 substituents independently selected from the group consisting of C₂₋₄ alkenyl, C₁₋₄ alkoxy, C₁₋₉ amide, C₁₋₅ oxycarbonyl, halo, hydroxy, nitro, and oxo.

The term “optionally substituted C₇₋₁₂ bicycloalkylene” refers to a bivalent C₇₋₁₂ bicycloalkylene, for example one that is attached to both R₉ and R₁₁ of formula I, optionally having 1 to 3 substituents independently selected from the group consisting of C₂₋₄ alkenyl, C₁₋₄ alkoxy, C₁₋₉ amide, C₁₋₅ oxycarbonyl, halo, hydroxy, nitro, and oxo.

The term “C₁₋₅ oxycarbonyl” refers to a carboxy group (—CO₂H) and C₁₋₄ alkyl ester thereof.

The term “C₃₋₈ cycloalkyl” refers to an alkyl ring having from three to eight carbon atoms, and includes cyclopropyl, cyclobutyl, cyclopentyl, cyclohexyl, and the like.

The term “optionally substituted C₃₋₈ cycloalkyl” refers to a C₃₋₈ cycloalkyl optionally having from 1 to 6 substituents independently selected from the group consisting of optionally substituted C₁₋₄ alkyl, C₂₋₄ alkenyl, C₁₋₄ alkoxy, C₁₋₉ amide, aminoC₁₋₈ alkyl, C₁₋₅ oxycarbonyl, cyano, C₃₋₈ cycloalkyl, C₃₋₈ cycloalkoxy, halo, hydroxy, nitro, oxo, and optionally substituted phenyl.

The term “C₃₋₈ cycloalkoxy” refers to a C₃₋₈ cycloalkyl attached through an oxygen atom.

The term “optionally substituted C₃₋₈ cycloalkylene” refers to a bivalent C₃₋₈ cycloalkyl, for example one that is attached to both R₉ and R₁₁ of formula I, optionally having from 1 to 6 substituents independently selected from the group consisting of optionally substituted C₁₋₄ alkyl, C₂₋₄ alkenyl, C₁₋₄ alkoxy, C₁₋₉ amide, aminoC₁₋₈ alkyl, C₁₋₅ oxycarbonyl, cyano, C₃₋₈ cycloalkyl, C₃₋₈ cycloalkoxy, halo, hydroxy, nitro, oxo, and optionally substituted phenyl.

The terms “halogen” and “halo” refers to a chloro, fluoro, bromo or iodo atom.

The term “heteroC₃₋₆ cycloalkyl” refers to a 4 to 10 membered monocyclic saturated ring having three to six ring carbon atoms and having one or more heteroatoms selected from the group consisting of nitrogen, oxygen, and sulfur. For example, but not limiting, the term includes azetidine, pyrrolidine, piperidine, piperazine, morpholine, and the like.

The term “optionally substituted heteroC₃₋₆ cycloalkyl” refers to a heteroC₃₋₆ cycloalkyl optionally substituted on the ring carbons with 1 to 4 substituents independently selected from the group consisting of optionally substituted C₁₋₄ alkyl, C₂₋₄ alkenyl, C₁₋₄ alkoxy, C₁₋₉ amide, aminoC₁₋₈ alkyl, C₁₋₅ oxycarbonyl, cyano, C₃₋₈ cycloalkyl, C₃₋₈ cycloalkoxy, halo, hydroxy, nitro, oxo, and optionally substituted phenyl; and optionally substituted on any ring nitrogen with a substituent selected from the group consisting of optionally substituted C₁₋₄ alkyl, C₂₋₄ alkenyl, C₃₋₈ cycloalkyl, and optionally substituted phenyl.

The term “optionally substituted heteroC₃₋₆ cycloalkylene” refers to a bivalent heteroC₃₋₆ cycloalkylene, for example one that is attached to both R₁₀ and R₁₂ of formula I, optionally substituted on the ring carbons with 1 to 4 substituents independently selected from the group consisting of optionally substituted C₁₋₄ alkyl, C₂₋₄ alkenyl, C₁₋₄ alkoxy, C₁₋₉ amide, aminoC₁₋₈ alkyl, C₁₋₅ oxycarbonyl, cyano, C₃₋₈ cycloalkyl, C₃₋₈ cycloalkoxy, halo, hydroxy, nitro, oxo, and optionally substituted phenyl; and optionally substituted on any ring nitrogen with a substituent selected from the group consisting of optionally substituted C₁₋₄ alkyl, C₂₋₄ alkenyl, C₃₋₈ cycloalkyl, and optionally substituted phenyl.

The term “heteroC₃₋₁₂ bicycloalkyl” refers to a three to fourteen membered bicyclic saturated rings having three to twelve ring carbon atoms and having one or more ring atoms selected from the group consisting of nitrogen, oxygen, and sulfur.

The term “optionally substituted heteroC₃₋₁₂ bicycloalkylene” refers to a bivalent heteroC₃₋₁₂ bicycloalkyl, for example one that is attached to both R₉ and R₁₁ of formula I, optionally substituted on the ring carbons with 1 to 4 substituents independently selected from the group consisting of optionally substituted C₁₋₄ alkyl, C₂₋₄ alkenyl, C₁₋₄ alkoxy, C₁₋₉ amide, aminoC₁₋₈ alkyl, C₁₋₅ oxycarbonyl, cyano, C₃₋₈ cycloalkyl, C₃₋₈ cycloalkoxy, halo, hydroxy, nitro, oxo, and optionally substituted phenyl; and optionally substituted on any ring nitrogen with a substituent selected from the group consisting of optionally substituted C₁₋₄ alkyl, C₂₋₄ alkenyl, C₃₋₈ cycloalkyl, and optionally substituted phenyl.

The term “heteroC₁₋₁₀ aryl” refers to a five to twelve membered monoyclic and polycyclic unsaturated, conjugated ring having aromatic character and having one to ten carbon atoms and one or more heteroatoms selected from the group consisting of nitrogen, oxygen, and sulfur. For example, but not limiting, the term includes azepine, diazepine, furan, thiophene, imidazole, isothiazole, isoxazole, oxadiazole, oxazole, oxadiazole, pyrazine, pyrazole, pyridazine, pyridine, pyrimidine, pyrroline, thiazole, thiadiazole, triazole, tetrazole, benzazepine, benzodiazepine, benzofuran, benzothiophene, benzimidazole, imidazopyridine, quinazoline, thienopyridine, indolizine, imidazopyridine, quinoline, isoquinoline, indole, benzoxazole, benzopyrazole, benzothiazole, and the like.

The term “optionally substituted heteroC₁₋₁₀ aryl” refers to a heteroC₁₋₁₀ aryl optionally having 1 to 5 substituents independently selected from the group consisting of C₁₋₄ alkyl, C₁₋₄ alkoxy, cyano, halogen, hydroxyl, nitro, oxo, and trifluoromethyl.

The term “optionally substituted heteroC₁₋₁₀ arylene” refers to a bivalent heteroC₁₋₁₀ arylene, for example one that is attached to both R₁₀ and R₁₂ of formula I, optionally substituted on the ring carbons with 1 to 4 substituents independently selected from the group consisting of optionally substituted C₁₋₄ alkyl, C₂₋₄ alkenyl, C₁₋₄ alkoxy, C₁₋₉ amide, aminoC₁₋₈ alkyl, C₁₋₅ oxycarbonyl, cyano, C₃₋₈ cycloalkyl, C₃₋₈ cycloalkoxy, halo, hydroxy, nitro, oxo, and optionally substituted phenyl; and optionally substituted on any ring nitrogen with a substituent selected from the group consisting of optionally substituted C₁₋₄ alkyl, C₃₋₈ cycloalkyl, and optionally substituted phenyl.

The term “optionally substituted heteroC₁₋₁₀ aryloxy” refers to a heteroC₁₋₁₀ aryl attached through an oxygen atom and having 1 to 5 substituents independently selected from the group consisting of C₁₋₄ alkyl, C₁₋₄ alkoxy, cyano, halogen, hydrogen, hydroxyl, nitro, and trifluoromethyl.

The term “a substituent convertible in vivo to hydrogen” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, and sulfonyl groups.

The term “oxo” refers to an oxygen atom having a double bond to the carbon to which it is attached to form the carbonyl of a ketone or aldehyde.

The term “optionally substituted phenyl” refers to a phenyl group optionally having 1 to 5 substituents independently selected from the group consisting of amino, C₂₋₄ alkenyl, C₁₋₄ alkyl, C₁₋₄ alkoxy, C₁₋₉ amide, aminoC₁₋₈ alkyl, C₁₋₅ oxycarbonyl, cyano, halogen, hydrogen, hydroxyl, nitro, and trifluoromethyl.

The term “C₁₋₆ sulfonyl” refers to a sulfonyl linked to a C₁₋₆ alkyl group or an optionally substituted phenyl.

The term “C₁₋₃ sulfonyl” refers to a —SO₂—C₁₋₃ alkyl group.

The term “pharmaceutically acceptable salt” refers to salts of pharmaceutically acceptable organic acids and bases or inorganic acids and bases. Such salts are well known in the art and include those described in Journal of Pharmaceutical Science, 66, 2-19 (1977). Examples are the hydrochloride and mesylate salts.

Generally combinations of the substituents in the present invention result in stable compounds. Here the term stable means compounds that are not substantially altered by production, recovery, and storage. Excluded from the groups described herein, unless otherwise noted, are ketal, hemiketals, aminals, hemiaminals, ketenes, and the like.

One of skill in the art will recognize that for the bivalent radicals herein a number of variations of attachment are possible. All such attachments of the present bivalent radicals are contemplated by the present invention.

The skilled artisan will appreciate that certain of the compounds of the present invention exist as isomers. All mixtures of stereoisomers and diastereomers, in any ratio, and specific stereoisomers and diastereomers of the compounds of the invention are contemplated to be within the scope of the present invention.

The term “compounds of the invention” include the embodiment of formula I and the embodiments and examples described herein.

In one embodiment the compounds of the invention include a compound of formula I wherein R₈ is optionally substituted 1,4-phenylene counting from the point of attachment to the nitrogen bearing R₈ being the 1-position wherein one substituent is 3-fluoro or 3-chloro. Such compounds are believed to advantageously provide activity against multi-drug resistant tumors.

Another embodiment relates to any of the embodiments above wherein R₂ is hydrogen.

Another embodiment relates to any of the embodiments above wherein R₃ is selected from the group consisting of hydrogen, halo, cyano, trifluoromethyl, hydroxy substituted C₁₋₄ alkyl, C₁₋₉ amide, and C₁₋₅ oxycarbonyl.

Another embodiment relates to any of the embodiments above wherein W is N.

Another embodiment relates to any of the embodiments above wherein R₁ is hydrogen.

Another embodiment relates to any of the embodiments above wherein R₈ is optionally substituted C₄₋₁₂ arylene.

Another embodiment relates to any of the embodiments above or below wherein R₈ is optionally substituted C₄₋₁₂ aryl wherein one substituent is C₁₋₄ alkoxy.

Another embodiment relates to any of the embodiments above or below wherein R₉ is —CONJ₉-, particularly wherein J₉ is hydrogen.

Another embodiment relates to any of the embodiments above or below wherein R₁₀ is selected from the group consisting of optionally substituted C₃₋₈ cycloalkylene and optionally substituted heteroC₃₋₆ cycloalkylene, particularly wherein R₁₀ is C₃₋₈ cycloalkylene.

Another embodiment relates to any of the embodiments above or below wherein R₁₁ is selected from the group consisting of C₁₋₄ alkylene, optionally substituted C₃₋₈ cycloalkylene, and optionally substituted heteroC₃₋₆ cycloalkylene, particularly wherein R₁₁ is heteroC₃₋₆ cycloalkylene.

Another embodiment relates to any of the embodiments above or below wherein R₈ is optionally substituted C₄₋₁₂ arylene wherein one substituent is halo.

Another embodiment relates to any of the embodiments above or below wherein R₈ is optionally substituted C₄₋₁₂ arylene wherein one substituent is fluoro.

Another embodiment relates to any of the embodiments above or below wherein R₆ is selected from the group consisting of optionally substituted C₁₋₆ alkyl and optionally substituted C₃₋₈ cycloalkyl.

Another embodiment relates to any of the embodiments above or below wherein R₆ is selected from the group consisting of C₁₋₆ alkyl and C₃₋₈ cycloalkyl.

In an even more specific embodiment relates to any of the embodiments above or below wherein R₈ is optionally substituted 1,4-phenylene counting from the point of attachment to the nitrogen bearing R₈ being the 1-position wherein one substituent is 3-fluoro or 3-chloro.

Another embodiment relates to any of the embodiments above wherein one of R₄ or R₅ is not hydrogen, (C₁₋₁₀)alkyl, or (C₃₋₁₂)cycloalkyl.

Another embodiment is the compound 4-(7-cyano-6-ethyl-5-isopropyl-5,6-dihydroimidazo[1,5-f]pteridine-3-ylamino)-N-(4-(4-cyclopropylmethyl)piperazin-1-yl)cyclohexyl-2-fluoro-5-methoxybenzamide.

The present compounds have advantageous pharmacodynamic and pharmacokinetic properties, such as bioavailability. In particular, the compound 4-(R)-7-cyano-6-ethyl-5-isopropyl-5,6-dihydroimidazo[1,5-f]pteridin-3-ylamino)-N-((1r,4R)-4-(4-(cyclopropylmethyl)piperazin-1-yl)cyclohexyl)-2-fluoro-5-methoxybenzamide advantageously provides activity against multi-drug resistant tumors and has advantageous pharmacodynamic and pharmacokinetic properties, such as bioavailability.

The compounds of the invention can be prepared by a variety of procedures, some of which are described below. All substituents, unless otherwise indicated, are as previously defined. The products of each step can be recovered by conventional methods including extraction, evaporation, precipitation, chromatography, filtration, trituration, crystallization, and the like.

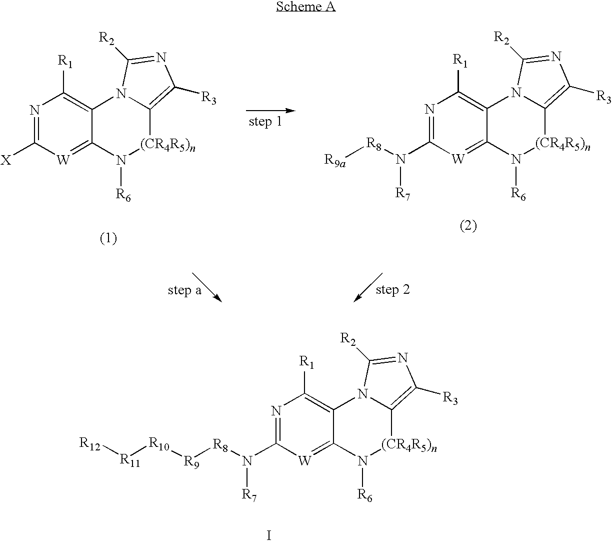

Scheme A, step 1, depicts the reaction of an appropriate compound of formula (1) with an appropriate compound of formula R₉ₐ—R₈—NH—R₇ to give a compound of formula (2). An appropriate compound of formula (1) is one in which X is a leaving group, such as chloro or bromo. Compounds of formula (1) are known in the art and are readily prepared from amides of the formula below:

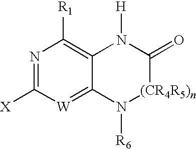

which are themselves well known, for example, see US 2006/0176380 and WO 2008/076392. An appropriate compound of formula R₉ₐ—R₈—NH—R₇ is one in which R₇ and R₈ are as required in the final compound of formula I or give rise to R₇ and R₈ as desired and R₉ₐ ultimately gives rise to R₉ in formula I and may be a protected R₉. The use and selection of protecting groups is well known and appreciated in the art. See for example, Protecting Groups in Organic Synthesis, Theodora Greene (Wiley-Interscience)).

While the reaction is discussed as using a compound of the formula R₉ₐ—R₈—NH—R₇ it is understood that the reaction can be carried out with other reagents, such as R₁₂—R₁₁—R₁₀—R₉—R₈—NH—R₇ as depicted in Scheme A, step a and discussed below. Other approaches are contemplated as well. For example, a group transferring what will give rise to R₁₁—R₁₀—R₉—R₈—NH—R₇, either in one step or in two or more steps, followed by introduction of the group R₁₂. A variety of other alternatives to introduce the groups group R₁₂, R₁₁, R₁₀, R₉, R₈, and R₇ are readily contemplated by the skilled person.

For example, a compound of formula (1) is reacted with an appropriate compound of formula R₉ₐ—R₈—NH—R₇. The reaction is carried out in a solvent, such as lower alcohol. The reaction is carried out with the use of a suitable acid, such as aqueous hydrochloric acid. The reaction typically is carried at temperatures of from about 80° C. to 100° C. The reaction typically requires 10 to 48 hours.

Alternately, for example, a compound of formula I is reacted with a compound of formula R₉ₐ—R₈—NH—R₇ without a solvent, or in a solvent such as, sulpholane, dimethylformamide, dimethyl acetamide, N-methylpyrrolidone, dimethylsufloxide, dioxane, and the like. The reaction typically is carried at temperatures of from about 80° C. to 200° C. The reaction typically requires 1 to 48 hours.

In yet another variation a compound of formula I is reacted with a compound of the formula R₉ₐ—R₈—NH—R₇ using Buchwald conditions. For example, the reaction carried out in solvent, such as dioxane and dimethylacetamide. The reaction may be carried out using a palladium catalyst such palladium acetate, with our without a ligand, such as xanthophos. The reaction is carried out using an excess of a base such as cesium carbonate. The reaction may be facilitated by microwave irradiation. The reaction is typically carried out at temperatures of from about 120° C. to 200° C. and generally requires about 10 minutes to 2 hours.

In an optional step, not shown, a protected compound of formula (2) is deprotected.

Scheme A, step 2, depicts the reaction of a compound of formula (2) with a compound that introduces R₁₂—R₁₁—R₁₀. Such reactions generally involve amide formations, sulfonation reactions, and alkylation reactions that are will understood in the art.

In an example of an amidation reaction a compound of formula (2) in which R₉ₐ is a carboxylic acid is reacted with a compound of the formula R₁₂—R₁₁—R₁₀—NHR₇ to give a compound of formula I in which R₉ is —CONJ₉-. Standard amide forming conditions are well known, including those using coupling agents, including those used in peptide couplings, such as 2-(1H-7-azabenzotriazol-1-yl)-1,1,3,3-tetramethyl uronium hexafluorophosphate methanaminium (HATU), dicyclohexylcarbodiimide (DCC), and 1-(3-dimethylaminopropyl)-3-ethylcarbodiimide hydrochloride. Such reactions are generally carried out using a base, such as N-methylmorpholine or triethylamine, in a suitable solvent such as dichloromethane, dimethylformamide (DMF), THF, and the like. If necessary or desired, an additive such as 4-(dimethylamino)pyridine, 1-hydroxybenzotriazole, and the like may be used to facilitate the reaction. Alternatively, other carboxylic acid equivalents, including acylating agents, such as an appropriate acylimidazole or a mixed anhydride or an appropriate acid halide may be reacted with the amine. It is readily understood that the same methodology can be used to prepare compound in which R₉ is —NJ₉CO— by starting with a compound of formula (2) in which R₉ₐ is an amine (having the group —NJ₉) with a compound of the formula R₁₂—R₁₁—R₁₀—CO₂H.

In an analogous manner compounds of formula I in which R₉ is —SO₂NJ₉- and —NJ₉ SO₂— can be formed by the reaction of sulfonyl halides with amines.

Alternately, a compound of formula I can be prepared directly as depicted in Scheme A, step a, by reacting a compound of formula I with a compound of formula R₁₂—R₁₁—R₁₀—R₉—R₈—NH—R₇. Such reactions are well known in the art. See WO 2006/021548. For example, the reaction carried out in solvent, such as dioxane and dimethylacetamide. The reaction may be carried out using a palladium catalyst such palladium acetate, with our without a ligand, such as xanthophos. The reaction is carried out using an excess of a base such as cesium carbonate. The reaction may be facilitated by microwave irradiation. The reaction is typically carried out at temperatures of from about 120° C. to 200° C. and generally requires about 10 minutes to 2 hours. The use of protecting groups may be beneficial in introducing such a group and if so can be readily employed by the skilled person.

In an additional optional step, not shown, a racemic mixture is resolved to give isomerically pure product. The specific pure stereoisomers and diasteromers of compounds of the invention can be prepared by a variety of methods known to the skilled person, such as resolution of mixtures and stereoselective synthesis by using stereochemically pure starting materials. The specific isomers of either starting materials or compounds of the invention can be obtained by techniques well known in the art, such as those found in Asymmetric Synthesis, edited by James P. Morrison (Academic Press 1983), Stereochemistry of Organic Compounds, E. I. Eliel and S. H. Wilen (Wiley 1994) and Enantiomers, Racemates, and Resolutions, J. Jacques, A. Collet, and S. H. Wilen (Wiley 1991), and those found in WO 97/25983 and WO 99/04778 such as chromatography, including on chiral stationary phases, enzymatic resolutions, or resolution of diastereomers formed for that purpose, including fractional crystallization of diastereomeric salts. As used herein the term “isomerically pure” refers to greater than 80%, preferably greater than 90%, more preferably greater than 95%, most preferably greater than 97% of a particular isomer.

It is also understood that the product of the present process may be used as a pharmaceutically acceptable salts which would be formed in an optional step, not shown. The formation of such salts is well known and readily practiced by the skilled person.

The present invention is further illustrated by the following examples and preparations. The terms and abbreviations used in the examples have their usual meaning unless otherwise indicated. Where indicated products of the preparations and examples were purified by chromatograph. Reverse phase chromatography was typically carried out on Waters Sunfire C-18, 5 μm, 30 mm×75 mm column or Phenomenex Gemini C-18 30 mm×75 mm AXIA column eluted with gradients of either 0.035% TFA in acetonitrile and 0.05% TFA in water or 10 mM ammonium bicarbonate in water and 10 mM ammonium bicarbonate in 20/80 water/acetonitrile. After isolation by reverse phase chromatography the product was typically obtained by evaporation of the solvent or evaporation of the solvent dilution with water adjusting the pH to basic, for example with saturated aqueous sodium carbonate, and extraction into an organic phase, followed by separation, drying, filtration, and evaporation. Normal phase chromatography was typically carried out using a NH-silica column eluted with gradients of ethyl acetate and hexanes. After isolation by normal phase chromatography the product was typically obtained by evaporation of the solvent. These examples and preparations are illustrative only and are not intended to limit the invention in any way.

### Preparation 1: 4-(5-Cyclopentyl-7-(ethoxycarbonyl)-5,6-dihydroimidazo[1,5-f]pteridin-3-ylamino)-3-methoxybenzoic acid

To a stirred solution of ethyl isocyanoacetate (327.8 μL, 3.0 mmol) in THF (5 mL) at −78° C. in dry ice-acetone bath, was added 1.5 M lithium diisopropylamide (2.0 mL, 3.0 mmol) in THF drop wise. The resulting solution was stirred for one hour. Meanwhile, sodium hydride (60% dispersion in mineral oil) (96 mg, 2.4 mmol) was added in portions to a stirred solution of 2-chloro-8-cyclopentyl-7,8-dihydropteridin-6(5H)-one (504 mg, 2.0 mmol) in THF (15 mL) under N₂ atmosphere in another round bottom flask. This reaction mixture was stirred at ambient temperature for 30 min. then cooled to −30° C. Diethyl chloro phosphate (346 μL, 2.4 mmol) was added drop wise, then slowly allowed to warm up to room temperature stirred for 30 min. Then cooled back to −30° C. and then transferred through cannula into the ethyl isocyanoacetate solution flask at −78° C. After 30 min., slowly allow to warm up to room temperature continued stirring for another one hour. After 2.0 hours, the reaction mixture was quenched with acetic acid (3.0 mmol), then evaporated under reduced pressure. Dissolved in CHCl₃ (75 mL) and washed with water, dried over Na₂SO₄ and evaporated. The crude reddish solid was purified by reverse phase chromatography to give ethyl 3-chloro-5-cyclopentyl-5,6-dihydroimidazo[1,5-f]pteridine-7-carboxylate. ¹H NMR (400 MHz, DMSO-d₆) δ ppm 1.31 (t, J=7.07 Hz, 3H) 1.50-1.98 (m, 8H) 4.26 (q, J=7.07 Hz, 2H) 4.92 (s, 2H) 5.01 (m, 1H) 8.60 (s, 1H) 8.70 (s, 1H). MS (ES) [M+H] calculated for C₁₆H₁₈ClN₅O₂, 348; found 348. A mixture of compound ethyl 3-chloro-5-cyclopentyl-5,6-dihydroimidazo[1,5-f]pteridine-7-carboxylate (0.3 g, 0.87 mmol), 4-amino-3-methoxybenzoic acid (217 mg, 1.3 mmol), isopropanol (25 mL), and concentrated hydrochloric acid (8 drops) was stirred at 90° C. for 20 h. The solid formed in the reaction mixture was filtered to give 0.21 g of the title compound. MS (ES) [M+H] found 479.

### Preparation 2: (R)-4-(5-Cyclopentyl-7-(ethoxycarbonyl)-6-ethyl-5,6-dihydroimidazo[1,5-f]pteridin-3-ylamino)-3-methoxybenzoic acid

To a stirred solution of ethyl isocyanoacetate (656 μL, 6.0 mmol) in THF (10 mL) at about −78° C. in dry ice-acetone bath was added 2.0 M lithium diisopropylamide (3.0 mL, 6.0 mmol) in THF drop wise. The resulting solution was stirred for one hour. Meanwhile, sodium hydride (60% dispersion in mineral oil) (192 mg, 4.8 mmol) was added in portions to a stirred solution of (R)-2-chloro-8-cyclopentyl-7-ethyl-7,8-dihydropteridin-6(5H)-one (1.12 g, 4.0 mmol) in THF (25 mL) under N₂ atmosphere in another round bottom flask. This reaction mixture was stirred at ambient temperature for 30 min. then cooled to −30° C. Diethyl chloro phosphate (692 μL, 4.8 mmol) was added drop wise, then slowly allowed to warm up to room temperature stirred for 30 min. Then cooled back to −30° C. and then transferred through cannula into ethyl isocyanoacetate solution flask at −78° C. After 30 min., slowly allowed to warm up to room temperature continued stirring for another one hour. After 2 hours, quenched with acetic acid 6.0 mmol), then evaporated the reaction mixture under reduced pressure. Dissolved this mixture in CHCl₃ (75 mL) and washed with water, dried over Na₂SO₄ and evaporated. The crude reddish was purified by reverse phase chromatography to give (R)-ethyl 3-chloro-5-cyclopentyl-6-ethyl-5,6-dihydroimidazo[1,5-f]pteridine-7-carboxylate. ¹H NMR (400 MHz, DMSO-d₆) δ ppm 0.59 (t, J=7.33 Hz, 3H) 1.31 (t, J=7.20 Hz, 3H) 1.42-1.71 (m, 2H) 1.76-2.05 (m, 8H) 4.12-4.40 (m, 3H) 5.44 (dd, J=5.94, 3.41 Hz, 1H) 8.61 (s, 1H) 8.77 (s, 1H). MS (ES) [M+H] found 376. A mixture of compound (R)-ethyl 3-chloro-5-cyclopentyl-6-ethyl-5,6-dihydroimidazo[1,5-f]pteridine-7-carboxylate (1.13 g, 3.0 mmol), 4-amino-3-methoxybenzoic acid (0.75 g, 4.5 mmol), isopropanol (25 mL), and concentrated hydrochloric acid (15 drops) was stirred at 90° C. for 20 h. The solid formed in the reaction mixture was filtered to give the title compound. ¹H NMR (400 MHz, DMSO-d₆) δ ppm 0.62 (t, J=7.45 Hz, 2H) 1.31 (t, J=7.07 Hz, 3H) 1.56-2.07 (m, 10H) 3.92 (s, 3H) 4.13-4.42 (m, 4H) 5.46 (m, 1H) 7.54-7.63 (m, 2H) 8.18 (br. s., 1H) 8.55 (s, 1H) 8.72 (s, 1H). MS (ES) [M+H] found 507.

### Preparation 3: Ethyl 3-chloro-5-cyclopentyl-6,7-dihydro-5H-imidazo[1,5-d]pyrimido[4,5-b][1,4]diazepine-8-carboxylate

A method analogous to Preparation 1 gave the title compound. ¹H NMR (400 MHz, DMSO-d₆) δ ppm 1.30 (t, J=7.45 Hz, 3H) 1.48-1.92 (m, 8H) 3.52 (br. s., 2H) 3.62-3.78 (m, 2H) 3.94 (s, 3H) 4.26 (q, J=7.16 Hz, 2H) 5.04 (quin, J=8.15 Hz, 1H) 7.46-7.69 (m, 2H) 8.14 (d, J=8.34 Hz, 1H) 8.29 (s, 1H) 8.47 (s, 1H) 9.46 (br. s., 1H). MS (ES) [M+H] found 362.

### Preparation 4: 4-(5-Cyclopentyl-8-(ethoxycarbonyl)-6,7-dihydro-5H-imidazo[1,5-d]pyrimido[4,5-b][1,4]diazepin-3-ylamino)-3-methoxybenzoic acid

A method analogous to Preparation 1 gave the title compound. ¹H NMR (400 MHz, DMSO-d₆) δ ppm 1.29 (t, J=7.45 Hz, 3H) 1.48-1.92 (m, 8H) 3.41-3.50 (m, 2H) 3.57-3.72 (m, 2H) 3.90-4.08 (m, 1H) 4.12-4.33 (m, 2H) 5.00 (quin, J=8.15 Hz, 1H) 8.12 (s, 1H) 8.46 (s, 1H). MS (ES) [M+H] found 493.

### Preparation 5: 4-(5-Cyclopentyl-8-(ethoxycarbonyl)-7,7-difluoro-6,7-dihydro-5H-imidazo[1,5-d]pyrimido[4,5-b][1,4]diazepin-3-ylamino)-3-methoxybenzoic acid

1H-Benzotriazole-1-methanol (51.0 g, 0.342 mol) was weighed into a round bottom flask and solubilized in EtOH (800 mL). Dibenzylamine (67.5 g, 0.342 mol) was added slowly (over 5 min) to the rapidly stirred solution. Formation of a white precipitate was observed shortly after starting addition. The solution was abandoned to stir for 24 h. At this time the reaction is judged complete by NMR (product fragments on LCMS to show only benzotriazole). The majority of the solvent was removed by rotovap and diethyl ether (1 L) was added to the residue with vigorous stirring. This mixture was filtered, the filtrand washed with ether and dried under vacuum to yield N-(dibenzylaminomethyl)benzotriazole as a fluffy white solid (112 g, quat. yield). ¹H NMR in CDCl₃: (400 MHz) δ ppm 3.80 (s, 4H) 5.48 (s, 2H) 7.21 (d, J=8.34 Hz, 1H) 7.34-7.43 (m, 1H) 7.49 (d, 1H) 8.09 (d, J=7.83 Hz, 1H). MS (ES) [M+H] found 329. To a suspension of zinc dust (2.7 g, 41.6 mmol) in dry THF (75 mL), stirred under argon atmosphere, was added chlorotrimethylsilane (2.63 mL, 20.8 mmol) followed, 10 min later, by ethyl dibromo-fluoroacetate (3.92 g, 20.8 mmol). After 10 min a slight exotherm was detected. The reaction was left to activate for 1 hour, whereupon it was cooled in an ice bath and a solution of N-(Dibenzylaminomethyl)benzotriazole (6.83 g, 20.8 mmol) in THF (50 mL) was added dropwise (over 30 minutes) and then the reaction mixture was allowed to warm to room temperature. After 18 h at r.t., NaHCO₃ (sat., 50 mL) was added, let stir for 20 minutes, the reaction was filtered on Celite, and the filter pad was washed with EtOAc. The layers were separated and the aqueous phase was extracted with EtOAc (3×50 mL). The organic layers were combined and washed with 1N HCl (70 mL), brine (70 mL), then dried over MgSO₄. After evaporation of the solvent, the residue was poured into rapidly stirring ether (100 mL); the solid formed was removed by filtration and discarded. The ether was evaporated from the filtrate to yield a dark yellow syrup. This crude residue was purified on silica gel column chromatographically (0-10% EtOAc:Hexanes) to yield ethyl 3-(dibenzylamino)-2,2-difluoropropanoate as a clear liquid (3.6 g, 50% yield). ¹H NMR in CDCl₃: (400 MHz) δ ppm 1.18 (t, J=7.07 Hz, 3H) 3.14 (t, J=13.26 Hz, 2H) 3.69 (s, 4H) 4.14 (q, J=7.16 Hz, 2H) 7.14-7.33 (m, 10H). MS (ES) [M+H] found 334.

In a round bottom flask, ethyl 3-(dibenzylamino)-2,2-difluoropropanoate (1.72 g, 5.2 mmol) was solubilized in EtOH (25 mL) and TFA added (0.4 mL, 5.5 mmol). Under an atmosphere of nitrogen Pd(OH)₂/C (170 mg of 20% Pd by wt. wet) was added. The reaction mixture was repeatedly purged with nitrogen and then left under hydrogen overnight. At this point the reaction was deemed complete by LCMS, filtered through a pad of Celite, the pad washed with EtOH and the filtrate concentrated without heating to yield ethyl 3-amino-2,2-difluoropropanoate-TFA salt as a foggy syrup which starts to crystallize upon standing (1.31 g, 94% yield). ¹H NMR in d₆-DMSO (400 MHz) δ ppm 1.29 (t, J=7.20 Hz, 3H) 3.72 (t, J=16.17 Hz, 2H) 4.34 (q, J=7.24 Hz, 2H). MS (ES) [M+H] found 154.

To a round bottom flask was added ethyl 3-amino-2,2-difluoropropanoate (1.31 g, 4.9 mmol), THF (50 mL), cyclopentanone (0.46 mL, 5.1 mmol), and NaOAc (400 mg, 4.9 mmol). To this mixture was added sodium triacetoxyborohydride (1.6 g, 7.3 mmol) portionwise over 15 minutes. The reaction was left to stir overnight. It was then added slowly to a stirring solution of ice (30 mL), NaHCO₃ (sat., 10 mL), and EtOAc (100 mL) cooled in an ice-salt bath. The layers were then separated and the aqueous pH further adjusted to 11 using 25% NaOH while cooling in the bath. The aqueous layer was washed with EtOAc (2×50 mL), the organic extracts combined, washed with cold NaHCO₃, (sat. 20 mL×2) brine (20 mL), dried over MgSO₄, filtered and concentrated to yield ethyl 3-(cyclopentylamino)-2,2-difluoropropanoate as a clear syrup (960 mg, 89%). ¹H NMR in d₆-DMSO (400 MHz) δ ppm 1.25 (t, J=7.07 Hz, 2H) 1.34-1.74 (m, 8H) 3.00 (q, 1H) 3.11 (t, J=14.15 Hz, 2H) 4.27 (q, J=7.07 Hz, 2H). MS (ES) [M+H] found 222.

Ethyl 3-(cyclopentylamino)-2,2-difluoropropanoate (396 mg, 1.79 mmol) was solubilized in acetone (40 mL, dry). The solution was cooled in an ice salt bath under a nitrogen atmosphere and K₂CO₃ (495 mg, 3.58 mmol) added. To this, a solution of 2,4-dichloro-5-nitropyrimidine (378 mg, 1.97 mmol) in acetone (10 mL, dry) was added dropwise. Upon completion of addition the reaction mixture abandoned and allowed to slowly warm to room temperature and stir overnight. The mixture was then filtered through paper, the filter pad washed with acetone, and the filtrate concentrated. The concentrate was then solubilized in EtOAc (10 mL). Hexanes (70 mL) were then added and the solution was put on the rotovap to slowly concentrate. Yellow crystals form and are collected by filtration to yield 360 mg of ethyl 3-((2-chloro-5-nitropyrimidin-4-yl)(cyclopentyl)amino)-2,2-difluoropropanoate (53%). The filtrate was found to contain a 1:1:1 mixture of the product, dichloronitropyrimidine, and the 2-addition product. This material was concentrated and set aside for future use. ¹H NMR in d₆-DMSO (400 MHz) δ ppm 1.22 (t, J=7.20 Hz, 3H) 1.36-1.98 (m, 8H) 3.64 (q, 1H) 4.25 (q, J=7.24 Hz, 2H) 4.35 (t, J=13.77 Hz, 2H) 8.94 (s, 1H). MS (ES) [M+H] found 379.

Ethyl 3-((2-chloro-5-nitropyrimidin-4-yl)(cyclopentypamino)-2,2-difluoropropanoate (1.0 g, 2.7 mmol) was dissolved in AcOH (10 mL) and then cooled in an ice bath. Iron powder (296 mg, 5.3 mmol) was added followed by the slow addition of HCl (1.5 mL, conc.). After 10 minutes the reaction was transferred to a heat bath and left to stir at 60° C. for 5 hours. The reaction was then cooled, the stir bar and unreacted iron removed by filtration through paper, and the solvent volume reduced by about 75% on a rotovap. The mixture was then diluted with ice water (15 mL) and EtOAc (20 mL), the layers separated, the aqueous layer washed with EtOAc (2×30 mL), the organic extracts combined and washed with sat. NaHCO₃ (20 mL), brine (20 mL), dried over MgSO₄, and filtered and concentrated to yield a brown syrup. Trituration with EtOAc (15 mL) and ether (100 mL) was used to coax out a light yellow solid to give 2-chloro-9-cyclopentyl-7,7-difluoro-8,9-dihydro-5H-pyrimido[5,4-b][1,4]diazepin-6(7H)-one (535 mg, 65% yield). ¹H NMR in d₆-DMSO (400 MHz) δ ppm 1.41-1.96 (m, 8H) 3.98 (t, J=11.49 Hz, 2H) 4.87 (q, 1H) 8.12 (s, 1H) 11.06 (br. s., 1H). MS (ES) [M+H] found 303.

A method analogous to Preparation 1 gave the title compound. ¹H NMR (400 MHz, DMSO-d₆) δ ppm 1.29 (t, J=8.00 Hz, 3H) 1.58 (br. s., 4H) 1.74 (br. s., 2H) 1.97 (br. s., 2H) 3.94 (m, 5H) 4.29 (q, J=7.07 Hz, 2H) 4.61 (t, J=8.08 Hz, 1H) 7.44-7.70 (m, 2H) 8.31 (d, J=8.34 Hz, 1H) 8.48 (s, 1H) 8.72 (br. s., 1H) 8.91 (s, 1H).

### Preparation 6: (R)-4-(5-Cyclopentyl-7-(ethoxycarbonyl)-6-ethyl-5,6-dihydroimidazo[1,5-f]pteridin-3-ylamino)-2-fluoro-5-methoxybenzoic acid

(R)-ethyl 3-chloro-5-cyclopentyl-6-ethyl-5,6-dihydroimidazo[1,5-f]pteridine-7-carboxylate (1.5 g, 4.0 mmol), 4-amino-2-fluoro-5-methoxybenzoic acid (888 mg, 4.8 mmol), palladium acetate (89.8 mg, 0.4 mmol), xanthophos (462.8 mg, 0.8 mmol), and cesium carbonate (5.2 g, 16 mmol) were dissolved in dioxane:dimethylacetamide (2:1, 20 mL) in a sealed cap glass microwave vessel and microwave irradiated under Buchwald coupling reaction at 160° C. for 30 minutes. Then added water and filtered through Celite® bed and acidified to pH=5 using 1 N HCl and extracted into EtOAc (2×150 mL) and evaporated under reduced pressure to a minimal amount and filtered the solid precipitated out using vacuum filtration and dried to give 1.45 grams of product (69%). ¹H NMR (400 MHz, DMSO-d₆) δ ppm 0.62 (t, J=7.45 Hz, 3H) 1.31 (t, J=7.07 Hz, 3H) 1.64 (d, J=5.31 Hz, 2H) 1.72-2.01 (m, 7H) 2.05-2.25 (m, 1H) 3.34 (br. s., 1H) 3.92 (s, 3H) 4.27 (q, J=6.99 Hz, 2H) 4.37-4.56 (m, 1H) 5.43 (dd, J=6.95, 3.16 Hz, 1H) 7.35 (d, J=6.82 Hz, 1H) 8.03 (s, 1H) 8.30 (d, J=13.64 Hz, 1H) 8.53 (s, 1H) 8.74 (s, 1H). MS (ES) [M+H] found 525.

### Preparation 7: (R)-4-(5-Cyclopentyl-7-(ethoxycarbonyl)-6-methyl-5,6-dihydroimidazo[1,5-f]pteridin-3-ylamino)-3-methoxybenzoic acid

A stirred solution of ethyl isocyanoacetate (2.6 mL, 23.67 mmol) in THF (20 mL) at −78° C. in dry ice-acetone bath, added 2.0 M lithium diisopropylamide (11.8 mL, 23.67 mmol) in THF drop wise. The resulting solution was stirred for one hour at this temperature. Meanwhile, sodium hydride (60% dispersion in mineral oil) (821 mg, 20.52 mmol) was added in portions to a stirred solution of (R)-2-chloro-8-cyclopentyl-7-methyl-7,8-dihydropteridin-6(5H)-one (4.2 g, 15.78 mmol) in THF (50 mL) under N₂ atmosphere in another round bottom flask. This reaction mixture was stirred at ambient temperature for 30 min. then cooled to −30° C. Diethyl chloro phosphate (2.72 mL, 18.93 mmol) was added drop wise, then slowly allowed to warm up to room temperature stirred for 30 min. Then cooled back to −30° C. and then transferred through cannula into ethyl isocyanoacetate solution flask at −78° C. After 30 min., slowly allowed to warm up to room temperature continued stirring for another one hour. After completing the reaction, quenched with acetic acid (21 mmol), then evaporated the reaction mixture under reduced pressure. Dissolved this mixture in CHCl₃ (200 mL) and washed with water, dried over Na₂SO₄ and evaporated. The crude reddish solid was purified by reverse phase chromatography to yield (R)-ethyl 3-chloro-5-cyclopentyl-6-methyl-5,6-dihydroimidazo[1,5-f]pteridine-7-carboxylate. ¹H NMR (400 MHz, CDCl₃) δ ppm 1.41-1.50 (m, 6H), 1.64-2.13 (m, 8H), 4.38-4.56 (m, 3H), 5.56 (q, J=6.57 Hz, 1H), 7.96 (s, 1H), 8.33 (s, 1H). MS (ES) [M+H] found 362. A mixture of compound (R)-ethyl 3-chloro-5-cyclopentyl-6-methyl-5,6-dihydroimidazo[1,5-f]pteridine-7-carboxylate (1.0 g, 2.78 mmol), 4-amino-3-methoxybenzoic acid (0.70 g, 4.16 mmol), isopropanol (15 mL), and concentrated hydrochloric acid (15 drops) was stirred at 90° C. for 20 h. The solid formed in the reaction mixture was filtered to give 0.85 g the title compound as white solid (62%). MS (ES) [M+H] found 493.

### Preparation 8: (R)-4-(7-Cyano-5-cyclopentyl-6-ethyl-5,6-dihydroimidazo[1,5-f]pteridin-3-ylamino)-3-methoxybenzoic acid

Lithium hexamethyldisilazide (LiHMDS) (1M in THF, 103 mL, 0.103 mol) was added to a solution of (R)-2-chloro-8-cyclopentyl-7-ethyl-7,8-dihydropteridin-6(5H)-one (26.32 g, 94.0 mmol) in THF at −50° C. under nitrogen. The reaction was allowed to warm to room temperature and stirred for 10 minutes. It was then cooled to −50° C. and diethyl chlorophosphate (14.8 mL, 17.8 g, and 0.103 mol) was added via syringe over about 2 min. After the addition was complete, the reaction mixture was brought to room temperature and stirred until greater than 90% conversion appeared to be achieved (about 1-2 h). This solution was cooled to −78° C., and a cold (−78° C.) solution of freshly prepared isocyanoacetonitrile was added. The reaction mixture was stirred for 2-3 min and tBuOK was added (50.0 g, 0.446 mol) in one portion with vigorous stirring. The reaction mixture was stirred at −78° C. for 20-30 min, poured into 1.4 L of cold water and stirred for 20 min. The resulting yellow precipitate was filtered, washed with water (2×100 mL) and dried in air (about 1 h). The resulting precipitate was dissolved in dichloromethane (about 700 mL), dried (MgSO4), filtered and concentrated in vacuo. The solid was diluted with ether (100 mL) and stirred vigorously until a fine suspension resulted. The solid was filtered and dried in vacuum to afford the title compound as a white solid (21.6 g, 70%). ¹H NMR (400 MHz, DMSO-d₆) δ ppm 0.73 (t, J=7.33 Hz, 3H) 1.46-1.71 (m, 2H) 1.76-2.06 (m, 8H) 4.28 (quin, J=8.40 Hz, 1H) 5.33 (t, J=5.68 Hz, 1H) 8.73 (s, 1H) 8.79 (s, 1H). MS (ES) [M+H], found 329. (R)-3-chloro-5-cyclopentyl-6-ethyl-5,6-dihydroimidazo[1,5-f]pteridine-7-carbonitrile (1.52 g, 4.62 mmol) and 4-amino-3-methoxybenzoic acid (0.850 g, 5.08 mmol) were dissolved in dioxane (25 mL) and pTSA was added (0.636 g, 3.69 mmol). The resulting suspension was stirred at 100° C. in a closed vial for 2 d and cooled to room temperature. It was diluted with ethyl acetate (50 mL) and the precipitate was filtered off, washed well with ethyl acetate (100 mL) and dried to afford the crude title compound as a light brown solid (2.12 g, quant). ¹H NMR (DMSO-d₆) δ: 9.50 (br. s., 1H), 8.88 (s, 1H), 8.70 (s, 1H), 8.11 (d, J=8.6 Hz, 1H), 7.65 (dd, J=8.3, 1.8 Hz, 1H), 7.58 (d, J=1.8 Hz, 1H), 5.43 (dd, J=6.4, 4.4 Hz, 1H), 4.44-4.59 (m, 1H), 3.93 (s, 3H), 1.82-1.40 (m, 10H), 0.73 (t, J=7.3 Hz, 3H); MS (ES) [M+H] found 460.

### Preparation 9: (R)-4-(7-Cyano-5-cyclopentyl-6-ethyl-5,6-dihydroimidazo[1,5-f]pteridin-3-ylamino)-2-fluoro-5-methoxybenzoic acid

In a microwave glass vessel (R)-3-chloro-5-cyclopentyl-6-ethyl-5,6-dihydroimidazo[1,5-f]pteridine-7-carbonitrile (984 mg, 3.0 mmol), 4-amino-2-fluoro-5-methoxybenzoic acid (666 mg, 3.6 mmol) were dissolved in 12 mL of 1:1 dimethyl acetamide (DMA) and dioxane mixture and added xanthophos (4,5-Bis(diphenylphosphino)-9,9-dimethylxanthene) (347 mg, 0.6 mmol), Cs₂CO₃ (3.91 g, 12.0 mmol) and Pd(OAc)₂ (67 mg, 0.3 mmol). The reaction was performed in a sealed tube and microwave irradiated at 160° C. for 20 min. The reaction mixture was diluted with 20 mL water and filtered through a Celite® bed under vacuum, and acidified to pH-5 with diluted HCl and extracted into ethyl acetate several times. The combined ethyl acetate was evaporated to a minimal volume and the resulted precipitated was collected by filtration gave the white solid product (1.02 g, 71%). ¹H NMR (400 MHz, DMSO-d₆) δ ppm 0.74 (t, J=7.33 Hz, 2H) 1.54-2.09 (m, 10H) 3.87 (s, 3H) 4.44 (quin, J=8.59 Hz, 1H) 5.19 (dd, J=8.08, 3.54 Hz, 1H) 7.31 (d, J=6.82 Hz, 1H) 7.97 (s, 1H) 8.10 (d, J=13.14 Hz, 1H) 8.65 (s, 1H) 8.74 (s, 1H); MS (ES) [M+H] found 478.

### Preparation 10.1: (R)-4-(7-Cyano-6-ethyl-5-isopropyl-5,6-dihydroimidazo[1,5-f]pteridin-3-ylamino)-3-methoxybenzoic acid

Lithium hexamethyldisilazide (1M in THF, 103 mL, 0.103 mol) was added to a solution of (R)-2-chloro-7-ethyl-8-isopropyl-7,8-dihydropteridin-6(5H)-one (23.9 g, 94.0 mmol) in THF at −50° C. under nitrogen. The reaction was allowed to warm to room temperature and stirred for 10 minutes. It was then cooled to −50° C. and diethyl chlorophosphate (14.8 mL, 17.8 g, and 0.103 mol) was added via syringe over about 2 min. After the addition was complete, the reaction mixture was brought to room temperature and stirred until greater than 90% conversion appeared to be achieved (about 1-2 h). This solution was cooled to −78° C., and a cold (−78° C.) solution of freshly prepared isocyanoacetonitrile was added. The reaction mixture was stirred for 2-3 min and tBuOK was added (50.0 g, 0.446 mol) in one portion with vigorous stirring. The reaction mixture was stirred at −78° C. for 20-30 min, poured into 1.4 L of cold water and stirred for 20 min. The resulting yellow precipitate was filtered, washed with water (2×100 mL) and dried in air (about 1 h). The resulting precipitate was dissolved in dichloromethane (about 700 mL), dried (MgSO₄), filtered and concentrated in vacuo. The solid was diluted with ether (100 mL) and stirred vigorously until a fine suspension resulted. The solid was filtered and dried in vacuum to afford (R)-3-chloro-6-ethyl-5-isopropyl-5,6-dihydroimidazo[1,5-f]pteridine-7-carbonitrile as a white solid (21.2 g, 75%). ¹H NMR (DMSO-d₆) δ: 8.79 (s, 1H), 8.72 (s, 1H), 5.33 (dd, J=6.4, 4.7 Hz, 1H), 4.35-4.48 (m, 1H), 1.77-1.88 (m, 2H), 1.40 (d, J=2.0 Hz, 3H), 1.38 (d, J=2.0 Hz, 3H), 0.71 (t, J=7.3 Hz, 3H); ESI-MS: m/z 303 (M+H)⁺ (R)-3-Chloro-6-ethyl-5-isopropyl-5,6-dihydroimidazo[1,5-f]pteridine-7-carbonitrile (1.40 g, 4.62 mmol) and 4-amino-3-methoxybenzoic acid (0.850 g, 5.08 mmol) were dissolved in dioxane (25 mL) and para-toluenesulfonic acid was added (0.636 g, 3.69 mmol). The resulting suspension was stirred at 100° C. in a closed vial for 2 d and cooled to room temperature. It was diluted with ethyl acetate (50 mL) and the precipitate was filtered off, washed well with ethyl acetate (100 mL) and dried to afford crude title compound as a light brown solid (2.00 g, quant). ¹H NMR (DMSO-d₆) δ: 9.50 (br. s., 1H), 8.88 (s, 1H), 8.70 (s, 1H), 8.11 (d, J=8.6 Hz, 1H), 7.65 (dd, J=8.3, 1.8 Hz, 1H), 7.58 (d, J=1.8 Hz, 1H), 5.43 (dd, J=6.4, 4.4 Hz, 1H), 4.44-4.59 (m, 1H), 3.93 (s, 3H), 1.82-1.95 (m, 2H), 1.41 (d, J=4.5 Hz, 3H), 1.40 (d, J=4.8 Hz, 3H), 0.73 (t, J=7.3 Hz, 3H); MS (ES) [M+H] found 434.

### Preparation 10.2: (R)-4-(7-Cyano-6-ethyl-5-isopropyl-5,6-dihydroimidazo[1,5-f]pteridin-3-ylamino)-3-methoxybenzoic acid

To a stirred solution of aminoacetonitrile hydrochloride (42 g, 454 mmol) in dichloromethane (450 mL), N,N-dimethylformamide dimethyl acetal (79 mL, 590 mmol) was added, followed by triethyl amine (96 mL, 681 mmol). After stirring for 4 hours at room temperature the solvent is reduced to about 10% its original volume in vacuo, the resulting slurry was diluted with diethyl ether (450 mL) and then filtered. The filtrate was then concentrated to yield a tan liquid. This liquid was then distilled at 100° C. at 60 mbar (6 kPa) to yield acetonitriledimethylamidine as a clear liquid (33 g, 65% yield) which was stored in a refrigerator under nitrogen.

LHMDS (1.0 M in hexanes, 72 mL, and 72 mmol) was added slowly to a solution of (R)-2-chloro-7-ethyl-8-isopropyl-7,8-dihydropteridin-6(5H)-one (16.6 g, 65.4 mmol) stirring in THF (250 mL) in an acetone:dry ice bath under nitrogen. Diethyl chlorophosphate (10.3 mL, 72 mmol) was then added over the course of 2 minutes. The reaction was allowed to stir in the cold bath for 30 minutes before removing the bath and allowing the reaction to warm to room temperature and stir for 1 hour.

The reaction mixture was again cooled in an acetone/dry ice bath under nitrogen. Freshly distilled acetonitriledimethylamidine was added slowly (14.5 g, 130.8 mmol), followed by LHMDS (1.0 M in hexanes, 131 mL, 131 mmol). After 45 minutes acetic acid was added (75 mL) and the solution heated to 70° C. for 20 minutes. The reaction was then cooled in an ice bath, filtered, and the filtrate poured into quickly stirring ice water (1.5 L). After stirring for 10 minutes, filtration yielded the product as a yellow solid which was then washed by sonication with diethyl ether and filtered to yield (R)-3-chloro-6-ethyl-5-isopropyl-5,6-dihydroimidazo[1,5-f]pteridine-7-carbonitrile as a cream colored solid (18 g, 92% yield).

The title compound was obtained using an analogue condition described in Preparation 10.1 from (R)-3-Chloro-6-ethyl-5-isopropyl-5,6-dihydroimidazo[1,5-f]pteridine-7-carbonitrile and 4-amino-3-methoxybenzoic acid in dioxane. ¹H NMR (DMSO-d₆) δ: 9.50 (br. s., 1H), 8.88 (s, 1H), 8.70 (s, 1H), 8.11 (d, J=8.6 Hz, 1H), 7.65 (dd, J=8.3, 1.8 Hz, 1H), 7.58 (d, J=1.8 Hz, 1H), 5.43 (dd, J=6.4, 4.4 Hz, 1H), 4.44-4.59 (m, 1H), 3.93 (s, 3H), 1.82-1.95 (m, 2H), 1.41 (d, J=4.5 Hz, 3H), 1.40 (d, J=4.8 Hz, 3H), 0.73 (t, J=7.3 Hz, 3H); MS (ES) [M+H] found 434.

### Preparation 11.1: (R)-4-(7-Cyano-6-ethyl-5-isopropyl-5,6-dihydroimidazo[1,5-f]pteridin-3-ylamino)-2-fluoro-5-methoxybenzoic acid

(R)-3-chloro-6-ethyl-5-isopropyl-5,6-dihydroimidazo[1,5-f]pteridine-7-carbonitrile (1.40 g, 4.62 mmol) and 4-amino-2-fluoro-3-methoxybenzoic acid (0.850 g, 5.08 mmol) were dissolved in dioxane (25 mL) and p-toluenesulfonic acid was added (0.636 g, 3.69 mmol). The resulting suspension was stirred at 100° C. in a closed vial for 2 days and cooled to room temperature. It was diluted with ethyl acetate (50 mL) and the precipitate was filtered off, washed well with ethyl acetate (100 mL) and dried to afford the crude title compound as white solid in 60-70% yield. ¹H NMR (DMSO-d₆) δ: 8.78 (s, 1H), 8.66 (s, 1H), 8.33 (d, J=13.6 Hz, 1H), 8.28 (br. s., 1H), 7.38 (d, J=6.8 Hz, 1H), 5.25-5.33 (m, 1H), 4.54-4.67 (m, 1H), 3.93 (s, 3H), 1.76-1.87 (m, 2H), 1.46 (d, J=6.8 Hz, 3H), 1.42 (d, J=6.8 Hz, 3H), 0.74 (t, J=7.3 Hz, 3H); MS (ES) [M+H] found 452.

### Preparation 11.2: (R)-4-(7-Cyano-6-ethyl-5-isopropyl-5,6-dihydroimidazo[1,5-f]pteridin-3-ylamino)-2-fluoro-5-methoxybenzoic acid

(R)-3-Chloro-6-ethyl-5-isopropyl-5,6-dihydroimidazo[1,5-f]pteridine-7-carbonitrile (1.06 g, 3.5 mmol), 4-amino-2-fluoro-3-methoxybenzoic acid (0.676 g, 3.65 mmol), palladium (II) acetate (76 mg, 0.348 mmol), 4,5-Bis(diphenylphosphino)-9,9-dimethylxanthene (0.405 g, 0.71 mmol), and cesium carbonate (4.55 g, 14.0 mmol) were combined in N,N-dimethylacetamide (15 mL) and sonicated to a fine suspension. Dioxane (5 mL) was added and the reaction mixture was degassed by 3 cycles of vacuum and nitrogen fill. The reaction vessel was capped and the mixture was microwaved at 160° C. for 15 minutes, cooled, diluted with water (10 mL) and brine (50 mL) and the pH was adjusted to about 2 with hydrochloric acid, to give precipitate. The precipitate was collected and washed with methanol and ethyl acetate to give about 1.03 g of the title compound.

### Preparation 12: 4-(4-Methylpiperazin-1-yl)cyclohexanamine (cis and trans isomers)

In a 100 mL flask, 4-acetamidocyclohexanone (300 mg, 1.93 mmol), N-methyl piperazine (387 g, 3.866 mmol) were dissolved in dry toluene (25 mL) and added methanesulfonic acid (MSA) (50 μL) and refluxed using Dean-Stark apparatus for five hours with occasional decanting toluene from Dean-Stark apparatus 3-4 times. Then removed half of the toluene from the reaction using Dean-Stark apparatus and cooled to 50° C. and added 25 mL ethanol then removed the Dean-Stark apparatus and cooled to 15-20° C. NaBH₄ (2.126 mmol) was then added portion wise under nitrogen atmosphere. Continued stirring the reaction for overnight, 2.0 mL of 4 N HCl was added drop wise to the reaction. The volatiles removed from the reaction under reduced pressure. The gummy solid reaction mixture was basified with 5.0 mL sat. K₂CO₃, diluted with another 5.0 mL water and further basified with 50% NaOH solution to pH of about 13. Then added EtOAc (50 mL) and filtered the solid product and washed with EtOAc and ether and collected N-(4-(4-methylpiperazin-1-yl)cyclohexyl)acetamide as white powder (328 mg, 71%). ¹H NMR (400 MHz, DMSO-d₆). ESI-MS: m/z 240 (M+H)⁺. N-(4-(4-methylpiperazin-1-yl)cyclohexyl)acetamide (328 mg, 1.372 mmol) was refluxed in 24% conc.HCl solution for 20 hours at 115° C. The solution was evaporated under reduced pressure and the solid product obtained was dissolved and recrystallized from isopropyl alcohol to get the product as white hydrochloride salt (175 mg). ESI-MS: m/z 198 (M+H)⁺.

### Preparation 13: 4-(4-Ethylpiperazin-1-yl)cyclohexanamine (cis and trans isomers)

In a 250 mL flask, 4-acetamidocyclohexanone (5.0 g, 32.2 mmol), N-ethyl piperazine (7.3 g, 64.4 mmol) were dissolved in dry toluene (125 mL) and added methanesulfonic acid (MSA) (1 mL) and refluxed using Dean-Stark apparatus for five hours with occasional decanting toluene from Dean-Stark apparatus 3-4 times. Half of the toluene was removed from the apparatus and the mixture was cooled to 50° C. and added 75 mL ethanol then removed the Dean-Stark apparatus and cooled to 15-20° C. NaBH₄ (35.4 mmol) was added portion wise under nitrogen atmosphere. Continued stirring the reaction for overnight. Then added 20 mL of 4 N HCl drop wise to the reaction. Removed volatiles from the reaction under reduced pressure. Basified the gummy solid reaction mixture with 20 mL sat.K₂CO₃, diluted with another 20 mL water and further basified with 50% NaOH solution to pH about 13. Then added EtOAc (100 mL) and filtered the solid product and washed with EtOAc and ether and collected N-(4-(4-ethylpiperazin-1-yl)cyclohexyl)acetamide (cis and trans isomers) as white powder (7.1 g, 87%). ¹H NMR (400 MHz, DMSO-d6). ESI-MS: m/z 254 (M+H)⁺. N-(4-(4-ethylpiperazin-1-yl)cyclohexyl)acetamide (7.1 g, 28.1 mmol) was refluxed in 24% conc.HCl solution for 20 hours at 115° C. Then evaporated the solution under reduced pressure and the solid product was obtained was dissolved and recrystallized from isopropyl alcohol to get the product as white hydrochloride salt (8.1 g). ESI-MS: m/z 212 (M+H)¹.

### Preparation 14: 4-(4-Propylpiperazin-1-yl)cyclohexanamine (cis and trans isomers)

In a 250 mL flask, 4-acetamidocyclohexanone (4 g, 25.8 mmol), N-propyl piperazine (6.8 g, 53.1 mmol) were dissolved in dry toluene (125 mL) and added methanesulfonic acid (MSA) (835 μL) and refluxed using Dean-Stark apparatus for five hours with occasional decanting toluene from Dean-Stark apparatus 3-4 times. Then removed half of the toluene from the apparatus and cooled to 50° C. and added 75 mL ethanol then removed the Dean-Stark apparatus and cooled to 15-20° C. Added NaBH₄ (28.4 mmol) portion wise under nitrogen atmosphere. The reaction was continued for overnight. After 30 min, 20 mL of 4 N HCl was added drop wise to the reaction. The volatiles was removed from the reaction under reduced pressure. The gummy solid reaction mixture was basified with 20 mL sat.K₂CO₃, diluted with 20 mL water and further basified with 50% NaOH solution to pH about 13. Then added EtOAc (100 mL) and filtered the solid product and washed with EtOAc and ether and collected N-(4-(4-propylpiperazin-1-yl)cyclohexyl)acetamide (cis and trans isomers) as white powder (9.4 g, 73%). ESI-MS: m/z 268 (M+H)¹.

N-(4-(4-propylpiperazin-1-yl)cyclohexyl)acetamide (9.4 g, 35.3 mmol) was refluxed in 24% conc.HCl solution for 20 hours at 115° C. Then evaporated the solution under reduced pressure and the solid product obtained was dissolved and recrystallized from isopropyl alcohol to get the product as white hydrochloride salt (6.4 g). ESI-MS: m/z 226 (M+H)¹.

### Preparation 15: 4-(4-i-Propylpiperazin-1-yl)cyclohexanamine (cis and trans isomers)

In a 250 mL flask, 4-acetamidocyclohexanone (4 g, 25.8 mmol), N-i-propyl piperazine (6 g, 46.9 mmol) were dissolved in dry toluene (125 mL) and added methanesulfonic acid (MSA) (1 mL) and refluxed using Dean-Stark apparatus for five hours with occasional decanting toluene from Dean-Stark apparatus 3-4 times. Then removed half of the toluene from the apparatus and cooled to 50° C. and added 75 mL ethanol then removed the Dean-Stark apparatus and cooled to 15-20° C. Added NaBH₄ (35.4 mmol) portion wise under nitrogen atmosphere. Continued stirring the reaction for overnight. After 30 min 20 mL of 4 N HCl was added drop wise to the reaction. The volatiles removed from the reaction under reduced pressure. Basified the gummy solid reaction mixture with 20 mL sat. K₂CO₃, diluted with another 20 mL water and further basified with 50% NaOH solution to pH about 13. Then added EtOAc (100 mL) and filtered the solid product and washed with EtOAc and ether and collected cyclohexanamine N-(4-(4-i-propylpiperazin-1-yl)cyclohexyl)acetamide (cis and trans isomers) as white powder (5.2 g, 75.6%). ESI-MS: m/z 268 (M+H)⁺. N-(4-(4-propylpiperazin-1-yl)cyclohexyl)acetamide (5.2 g, 19.5 mmol) was refluxed in 24% conc.HCl solution for 20 hours at 115° C. Then evaporated the solution under reduced pressure and the solid product obtained was dissolved and recrystallized from isopropyl alcohol to get the product as white hydrochloride salt (6.7 g). ESI-MS: m/z 226 (M+H)⁺.

### Preparation 16: 4-(4-i-Butylpiperazin-1-yl)cyclohexanamine (cis and trans isomers)

In a 250 mL flask, 4-acetamidocyclohexanone (5.47 g, 35.2 mmol), N-i-butyl piperazine (10 g, 70.3 mmol) were dissolved in dry toluene (75 mL) and added methanesulfonic acid (MSA) (250 μL) and refluxed using Dean-Stark apparatus for five hours with occasional decanting toluene from Dean-Stark apparatus 3-4 times. Then removed half of the toluene from the apparatus and cooled to 50° C. and added 75 mL ethanol then removed the Dean-Stark apparatus and cooled to 15-20° C. Added NaBH₄ (35.2 mmol) portion wise under nitrogen atmosphere. Continued stirring the reaction for overnight. Then added NaBH₄ (50 mmols), after 30 min added 12 mL of 4 N HCl drop wise to the reaction. The volatiles were removed from the reaction under reduced pressure. Basified the gummy solid reaction mixture with 12 mL sat.K₂CO₃, diluted with another 12 mL water and further basified with 50% NaOH solution to pH about 13. Then added EtOAc (100 mL) and filtered the solid product and washed with EtOAc and ether and collected N-(4-(4-i-burtylpiperazin-1-yl)cyclohexyl)acetamide (cis and trans isomers) as white powder (5.3 g, 54%). ESI-MS: m/z 282 (M+H)⁺. N-(4-(4-propylpiperazin-1-yl)cyclohexyl)acetamide (5.2 g, 18.5 mmol) was refluxed in 24% conc.HCl solution for 20 hours at 115° C. Then evaporated the solution under reduced pressure and the solid product obtained was dissolved and recrystallized from isopropyl alcohol to get the product as white hydrochloride salt (5.2 g). ESI-MS: m/z 240 (M+H)⁺.

### Preparation 17: 1′-(Ethyl)-1,4′-bipiperidin-4-amine hydrochloride

A round bottom flask was charged with piperidin-4-one hydrochloride monohydrate (5 g, 33 mmol), acetonitrile (40 mL), bromomethane (4 g, 33 mmol), and sodium carbonate (10 g). This mixture was heated overnight at 85° C., then cooled, filtered and the filtrate concentrated. The product was purified by flash chromatography (160 g column, 0-7% 2N NH₃ in MeOH:DCM) to give 1-(ethyl)piperidin-4-one as a yellow liquid (3.6 g, 62% yield).

To a solution of 1-(ethyl)piperidin-4-one (500 mg, 3.78 mmol) and 4-(BOC amino)piperidine (757 mg, 3.78 mmol) in THF (25 mL) was added AcOH (5 drops) and sodium triacetoxyborohydride (1.2 g, 5.67 mmol). This solution was allowed to stir overnight. Water (40 mL) was then added to the reaction, the layers separated, and the organic discarded. The aqueous layer was basified with sodium carbonate (10 mL sat.) and the product extracted into EtOAc. The organic layer was dried over sodium sulfate, filtered and concentrated to give tert-butyl 1′-(ethyl)-1,4′-bipiperidin-4-ylcarbamate (1 g, 85% yield). MS (ES) [M+H] found 312. tert-Butyl 1′-(ethyl)-1,4′-bipiperidin-4-ylcarbamate (1 g, 3.2 mmol) was dissolved in MeOH (15 mL), HCl was added (2 mL conc.) and the mixture was heated to 70° C. for 3 hours. The mixture was then cooled and to give a solid which was filtered off, washed with diethyl ether, and dried to yield the title compound (990 mg, 96% yield) as a white solid. MS (ES) [M+H] found 212.

### Preparation 18: 1′-Cyclopropylmethyl-1,4′-bipiperidin-4-amine hydrochloride

A round bottom flask was charged with piperidin-4-one hydrochloride monohydrate (10 g, 65.1 mmol), acetonitrile (70 mL), bromomethylcyclopropane (8.79 g, 65.1 mmol), and sodium carbonate (20.7 g, 195.3 mmol). This mixture was heated overnight at 85° C., then cooled, filtered and the filtrate concentrated. The product was purified by flash chromatography (160 g column, 0-7% 2N NH₃ in MeOH:DCM) to give 1-(cyclopropylmethyl)piperidin-4-one as a yellow liquid (5.6 g, 56% yield). ¹H NMR (400 MHz, CDCl₃) δ ppm 0.09-0.17 (m, 2H) 0.52-0.59 (m, 2H) 0.85-0.97 (m, 1H) 2.38 (d, J=6.57 Hz, 2H) 2.49 (t, J=6.19 Hz, 4H) 2.84 (t, J=6.19 Hz, 4H). MS (ES) [M+H] calculated for C₃₀H₃₇N₉O₂, 556; found 556. MS (ES) [M+H] calculated for C₉H₁₅NO, 154; found 154.

To a solution of 1-(cyclopropylmethyl)piperidin-4-one (750 mg, 4.9 mmol) and 4-(BOC amino)piperidine (982 mg, 4.9 mmol) in THF (30 mL) was added AcOH (20 drops) and sodium triacetoxyborohydride (3.1 g, 14.7 mmol). This solution was allowed to stir overnight. Water (40 mL) was then added to the reaction, the layers separated, and the organic discarded. The aqueous layer was basified with sodium carbonate (10 mL sat.) and the product extracted into EtOAc. The organic layer was dried over sodium sulfate, filtered and concentrated to give tert-butyl 1′-(cyclopropylmethyl)-1,4′-bipiperidin-4-ylcarbamate (513 mg, 31% yield). MS (ES) [M+H] calculated for C₁₉H₃₅N₃O₂, 338; found 338. tert-Butyl 1′-(cyclopropylmethyl)-1,4′-bipiperidin-4-ylcarbamate (513 mg, 1.52 mmol) was dissolved in MeOH (15 mL), HCl was added (2 mL conc.) and the mixture was heated to 70° C. for 3 hours. The mixture was then cooled and the product was filtered off, washed with diethyl ether, and dried to yield the title compound (530 mg, quant. yield) as a white solid. MS (ES) [M+H] found 238.

### Preparation 19: 1′-Isobutyl-1,4′-bipiperidin-4-yl-4-amine hydrochloride

To a solution of 1-(isobutyl)piperidin-4-one (3.10 g, 20 mmol) and 4-(BOC amino)piperidine (4.0 g, 20 mmol) in THF (80 mL) was added AcOH (60 drops) and sodium triacetoxyborohydride (12.72 g, 60 mmol). This solution was allowed to stir 4 hours. Water (75 mL) was then added to the reaction, the layers separated, and the organic discarded. The aqueous layer was basified with sodium carbonate (100 mL sat.) and the product extracted into EtOAc. The organic layer was dried over sodium sulfate, filtered and concentrated to yield 1′-(cyclopropylmethyl)-1,4′-bipiperidin-4-amine hydrochloride (1.7 g, 25%). MS (ES) [M+H] found 340. tert-butyl 1′-isobutyl-1,4′-bipiperidin-4-ylcarbamate (1.7 g, 5 mmol) was dissolved in MeOH (30 mL), conc.HCl was added (3 mL) and the mixture was heated to 70° C. for 3 hours. The mixture was then cooled and the product was filtered off, washed with diethyl ether, and dried to yield 1′-isobutyl-1,4′-bipiperidin-4-amine hydrochloride (1.71 g, quant. yield) as a white solid. MS (ES) [M+H] found 240.

### Preparation 20: 1′-Methyl-1,4′-bipiperidin-4-amine

To a solution of 1-methylpiperidin-4-one (1.13 g, 10 mmol) and N—BOC piperidin-4-amine (4 g, 20 mmol) in THF (150 mL) was added AcOH (1 ml) and sodium triacetoxyborohydride (6.3 g, 30 mmol). This solution was allowed to stir overnight. Water (40 mL) was then added to the reaction. The aqueous layer was basified with sodium carbonate (20 mL sat.) and the product of the reaction was extracted into EtOAc. The organic layer was dried over sodium sulfate, filtered and concentrated to yield the crude Boc-protected intermediate which was treated with 50% TFA (30 ml) in DCM (50 ml) at 40° C. for 24 h. The mixture was basified with sat. K₂CO₃ and then was extracted with DCM. The organic layer was dried over sodium sulfate, then concentrated to yield 1′-methyl-1,4′-bipiperidin-4-amine (400 mg, 20% yield). ¹H NMR (400 MHz, MeOD) δ ppm 1.45-1.67 (m, 4H), 1.82-1.91 (m, 2H), 1.92-2.01 (m, 2H), 2.09 (td, J=12.13, 2.27 Hz, 2H), 2.25-2.43 (m, 6H), 2.86-3.07 (m, 5H). MS (ES) [M+H] found 198.

### Preparation 21: 1-(1-(Cyclopropylmethyl)piperidin-4-yl)azetidin-3-amine

To a solution of 1-(cyclopropylmethyl)piperidin-4-one (500 mg, 3.27 mmol) and 4-(BOC amino)azetidine (559 mg, 3.27 mmol) in THF (40 mL) was added AcOH (5 drops) and sodium triacetoxyborohydride (2.07 g, 9.8 mmol). This solution was allowed to stir 5 hours. Water (75 mL) was then added to the reaction. The aqueous layer was basified with sodium carbonate (100 mL sat.) and the product extracted into EtOAc. The organic layer was dried over sodium sulfate, filtered and concentrated to yield tert-butyl 1-(1-(cyclopropylmethyl)piperidin-4-yl)azetidin-3-ylcarbamate (509 mg, 55%). MS (ES) [M+H] found 310.

tert-Butyl 1-(1-(cyclopropylmethyl)piperidin-4-yl)azetidin-3-ylcarbamate (509 mg, 1.8 mmol) was dissolved in MeOH (30 mL), conc.HCl was added (3 mL) and the mixture was heated to 70° C. for 3 hours. The mixture was then cooled and the product was filtered off, washed with diethyl ether, and dried to yield the title compound as the hydrochloride salt (525 g, quant. yield) as a white solid. MS (ES) [M+H] found 210.

### Preparation 22: (R)-3-(7-Cyano-6-ethyl-5-isopropyl-5,6-dihydroimidazo[1,5-f]pteridin-3-ylamino)-4-methoxybenzoic acid

(R)-3-Chloro-6-ethyl-5-isopropyl-5,6-dihydroimidazo[1,5-f]pteridine-7-carbonitrile (1.51 g, 5.0 mmo) and 3-amino-4-methoxybenzoic acid (1.25 g, 7.5 mmol) were combined in iso-propanol (50 mL) and a catalytic conc.HCl was added. The resulting suspension was stirred overnight at 95° C., then cooled to room temperature and filtered to give the title compound which can be used without further purification, yield 1.42 g, 66%. MS ES [M+H] found 434.

### Preparation 23: 4-Amino-N-((1r,4r)-4-(4-(cyclopropylmethyl)piperazin-1-yl)cyclohexyl)-2-fluoro-5-methoxybenzamide

4-N-Boc aminocyclohexanone (10.7 g, 50 mmol) was slowly dissolved in 100 mL of 4N HCl in dioxane under nitrogen atmosphere and stirred at room temperature. After 6 hours, the reaction mixture was evaporated under reduced pressure to yield 4-aminocyclohexanone hydrochloride as a white powder.

To a mixture of 2-fluoro-5-methoxy-4-nitrobenzoic acid (6.45 g, 30 mmol), 4-aminocyclohexanone hydrochloride (5.36 g, 36 mmol), DIEA (16.02 mL, 90 mmol) in 75 mL of anhydrous DMF was added HATU (13.69 g, 36 mmol) in two portions. The reaction mixture was stirred at room temperature for 2.5 hours, then evaporated most of the DMF under reduced pressure and re-suspended the reaction mixture in EtOAc (100 mL), washed three times (3×75 mL) with water and finally with brine solution. The organic layer was dried over Na₂SO₄ and evaporated to give a residue which was purified on silica gel column eluting with 0-100% EtOAc in hexanes to 2-fluoro-5-methoxy-4-nitro-N-(4-oxocyclohexyl)benzamide, 5.65 g (64%). ¹H NMR (400 MHz, DMSO-d₆) δ ppm 1.71-1.86 (m, 1H) 2.01-2.19 (m, 1H) 2.22-2.41 (m, 1H) 2.43-2.61 (m, 5H) 3.95 (s, 3H) 4.26 (m, 1H) 7.46 (d, J=5.56 Hz, 1H) 8.01 (d, J=8.84 Hz, 1H) 8.66 (d, J=7.33 Hz, 1H). MS (ES) [M+H] found 311.

2-Fluoro-5-methoxy-4-nitro-N-(4-oxocyclohexyl)benzamide (3.72 g, 12 mmol), 1-N-cyclopropylmethylpiperazine (3.36 g, 24 mmol) were dissolved in toluene (50 mL) and methanesulfonic acid (MSA) (225 μL) was added and the mixture was refluxed with a Dean-Stark reflux condenser. Toluene from the Dean-Stark condenser was removed three times and added fresh toluene to the reaction. After 5 hours, the reaction mixture was evaporated in vacuo to give a residue which was re-dissolved in ethanol (30 mL) before adding NaBH₄ (12 mmol) in portions at 20-25° C. After stirring overnight, aqueous 4N HCl solution (5 mL) was added and the reaction mixture was evaporated before adding sat. K₂CO₃ solution (12 mL) and further basified with 50% NaOH solution to pH=13. The mixture was then extracted twice with EtOAc (2×75 mL) and finally washed with brine, dried over Na₂SO₄ and evaporated in vacuo to give crude product. Treated the residue with ether and filtered off the product as solid, and then purified on silica gel column eluting with 0-100% EtOAc in hexanes to obtain 2.85 g containing as the major component N-((1r,4r)-4-(4-(cyclopropylmethyl)piperazin-1-yl)cyclohexyl)-2-fluoro-5-methoxy-4-nitrobenzamide and a minor amount of the cis-product. MS (ES) [M+H] found 435.

The N-((1r,4r)-4-(4-(cyclopropylmethyl)piperazin-1-yl)cyclohexyl)-2-fluoro-5-methoxy-4-nitrobenzamide and minor cis-isomer obtained above (2.82 g, 6.5 mmol) were dissolved in 60 mL of methanol:acetic acid (5:1), de-gassed using vacuum and a nitrogen balloon and added 500 mg of 10% Pd—C, then hydrogenated using hydrogen gas balloon for 8 hours. After completion, the reaction mixture was filtered through celite and evaporated to give a residue which was dissolved in EtOAc and washed with sat.NaHCO₃ solution and finally with brine solution. The organic layer was dried and evaporated to yield 1.73 g of the title compound. ¹H NMR (400 MHz, DMSO-d₆) δ ppm 0.10 (d, J=4.04 Hz, 2H) 0.36-0.56 (m, 2H) 0.84 (t, J=12.25 Hz, 1H) 1.24-1.42 (m, 4H) 1.74-1.97 (m, 2H) 2.28 (m, 2H) 2.53-2.78 (m, 4H) 3.55 (m, 1H) 3.76 (s, 3H) 5.57 (s, 2H) 6.24-6.48 (m, 1H) 6.92-7.10 (m, 1H) 7.33 (t, J=7.07 Hz, 1H). MS (ES) [M+H] found 405.

### Preparation 24: (R)-3-Chloro-6-ethyl-5-isopropyl-5,6-dihydroimidazo[1,5-f]pteridine-7-carbonitrile

To a stirred solution of aminoacetonitrile HCl (42 g, 454 mmol) in dichloromethane (450 mL), N,N-dimethylformamide dimethyl acetal (79 mL, 590 mmol) was added, followed by triethylamine (96 mL, 681 mmol). After stirring for 4 hours at room temperature the solvent was reduced to about 10% its original volume in vacuo, the resulting slurry was diluted with diethyl ether (450 mL) and then filtered. The filtrate was then concentrated to yield a tan liquid. This liquid was then distilled at 100° C., at 100 mbar vacuum to yield N′-(cyanomethyl)-N,N-dimethylformimidamide as a clear liquid (33 g, 65% yield). ¹H NMR (400 MHz, CHLOROFORM-d) δ ppm 2.89 (br. s., 6H) 4.19 (s, 2H) 7.44 (s, 1H).

Lithium hexamethyldisilazide (1.0 M in hexanes, 72 mL, 72 mmol) was added slowly to a solution of the (R)-2-chloro-7-ethyl-8-isopropyl-7,8-dihydropteridin-6(5H)-one (16.6 g, 65.4 mmol) stirring in THF (250 mL) in an acetone:dry ice bath under nitrogen. Diethyl chlorophosphate (10.3 mL, 72 mmol) was then added over the course of 2 minutes. The reaction was allowed to stir in the cold bath for 30 minutes before removing the bath and allowing the reaction to warm to room temperature and stir for 1 hour. ESI-MS: m/z 391 [M+H].

The solution above was cooled in an acetone/dry ice bath under nitrogen. N′-(cyanomethyl)-N,N-dimethylformimidamide was added slowly (14.5 g, 130.8 mmol), followed by lithium hexamethyldisilazide (1.0 M in hexanes, 131 mL, 131 mmol). After 45 minutes acetic acid was added (75 mL) and the solution heated to 70° C. for 20 minutes. The reaction was then cooled in an ice bath, filtered, and the filtrate poured into ice water (1.5 L) with vigorous stirring. After 10 minutes, the mixture was filtered to yield a yellow solid which was then sonicated in diethyl ether and filtered to yield the title compound as a cream colored solid (18 g, 92% yield). ¹H NMR (400 MHz, DMSO-d₆) δ ppm 0.71 (t, 3H) 1.39 (dd, J=6.69, 1.89 Hz, 6H) 1.75-1.88 (m, 2H) 4.37-4.49 (m, 1H) 5.34 (dd, J=6.44, 4.67 Hz, 1H) 8.72 (s, 1H) 8.80 (s, 1H). ESI-MS: m/z 302 [M+H].

### Example 1

### 4-(R)-7-Cyano-6-ethyl-5-isopropyl-5,6-dihydroimidazo[1,5-f]pteridin-3-ylamino)-N-((1r,4R)-4-(4-(cyclopropylmethyl)piperazin-1-yl)cyclohexyl)-2-fluoro-5-methoxybenzamide

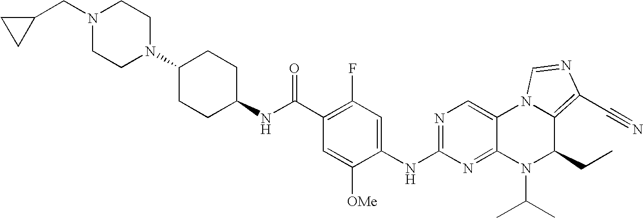

(R)-4-(7-Cyano-6-ethyl-5-isopropyl-5,6-dihydroimidazo[1,5-f]pteridin-3-ylamino)-2-fluoro-5-methoxybenzoic acid (104 mg, 0.230 mmol) and (1r,4r)-4-(4-(cyclopropylmethyl)piperazin-1-yl)cyclohexanamine hydrochloride (80.0 mg, 0.231 mmol) were dissolved in DMF (2 mL) and diisopropylethylamine (0.2 mL). HATU (100 mg, 0.289 mmol) was added and the reaction mixture was stirred for 2 h. It was diluted with water (10 mL) and basified with NaOH (50% aq.) to pH about 13. The resulting precipitate was filtered and dissolved in MeOH (2 mL) and purified using preparative reverse phase chromatography (eluting with a gradient of 20-40% 0.035% TFA/acetonitrile in 0.05% TFA/water). Product containing fractions were concentrated in vacuo to a small volume (about 3-5 mL), basified with NaOH (50% aq) to pH about 13 and extracted with EtOAc (3×3 mL). The combined organic extracts were dried (MgSO₄), filtered, concentrated in vacuo, chased with MeOH (3 mL) and the residue was crystallized with ether (3 mL). The product was filtered and dried in vacuum to afford the title compound as a white solid (47.5 mg, 31%). ¹H NMR (400 MHz, CHLOROFORM-d) δ ppm 0.10-0.19 (m, 2H) 0.47-0.59 (m, 2H) 0.78-0.96 (m, 4H) 1.18-1.38 (m, 3H) 1.38-1.57 (m, 8H) 1.75-1.88 (m, 1H) 1.88-2.07 (m, 3H) 2.21 (d, J=10.36 Hz, 2H) 2.26-2.42 (m, 3H) 2.41-2.99 (m, 8H) 3.87-4.02 (m, 4H) 4.73-4.87 (m, 1H) 5.09 (dd, J=8.34, 3.28 Hz, 1H) 6.62 (dd, J=15.03, 7.71 Hz, 1H) 7.58 (d, J=7.07 Hz, 1H) 7.86 (s, 1H) 7.96 (s, 1H) 8.34 (s, 1H) 8.44 (d, J=15.16 Hz, 1H). MS (ES) [M+H] found 671, m.p.: 251-255° C.

### Example 2

### 4-(R)-7-Cyano-5-cyclopentyl-6-ethyl-5,6-dihydroimidazo[1,5-f]pteridin-3-ylamino)-3-methoxy-N-(4-(4-methylpiperazin-1-yl)cyclohexyl)benzamide

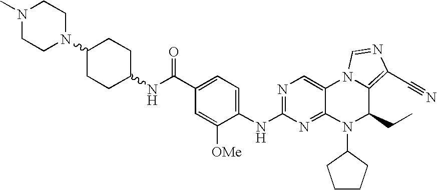

The title compound was prepared using a method analogous to Example 1 starting from (R)-4-(7-cyano-5-cyclopentyl-6-ethyl-5,6-dihydroimidazo[1,5-f]pteridin-3-ylamino)-3-methoxybenzoic acid and 4-(4-methylpiperazin-1-yl)cyclohexanamine. Final compounds (trans and cis-isomers) were separated using normal phase chromatography to give the product in two fractions. The faster fraction (minor): MS (ES) [M+H] found 639. The slower fraction (major): ¹H NMR (400 MHz, CDCl₃) δ ppm 0.86 (t, J=7.33 Hz, 3H), 1.25-1.35 (m, 2H), 1.38-1.54 (m, 2H), 1.70-1.93 (m, 11H), 2.17-2.30 (m, 7H), 2.48 (br. s., 3H), 2.63 (br. s., 4H), 3.85-4.02 (m, 4H), 4.50-4.66 (m, 1H), 4.96 (dd, J=8.97, 3.41 Hz, 1H), 5.95 (d, J=8.08 Hz, 1H), 7.23 (dd, J=8.59, 1.77 Hz, 1H), 7.43 (d, J=1.77 Hz, 1H), 7.77 (s, 1H), 7.94 (s, 1H), 8.31 (s, 1H), 8.49 (d, J=8.34 Hz, 1H), MS (ES) [M+H] found 639, m.p.: 187-192° C.

### Example 3

### 4-(R)-7-Cyano-5-cyclopentyl-6-ethyl-5,6-dihydroimidazo[1,5-f]pteridin-3-ylamino)-3-methoxy-N-(4-(4-ethylpiperazin-1-yl)cyclohexyl)benzamide

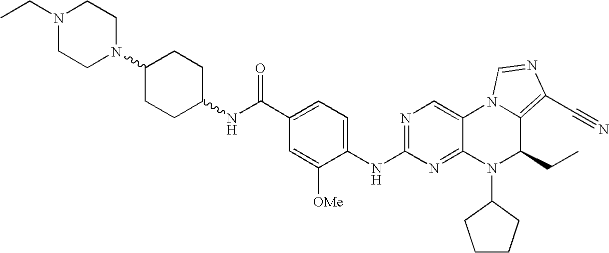

The title compound was prepared using a method analogous to Example 1 starting from (R)-4-(7-cyano-5-cyclopentyl-6-ethyl-5,6-dihydroimidazo[1,5-f]pteridin-3-ylamino)-3-methoxybenzoic acid and 4-(4-ethylpiperazin-1-yl)cyclohexanamine. The final compounds were separated using normal phase chromatography to give the product in two fractions. The faster fraction (minor): MS (ES) [M+H] found 653. Slower fraction (major): ¹H NMR (400 MHz, CDCl₃) δ ppm 0.86 (t, J=7.45 Hz, 3H), 1.10 (t, J=7.20 Hz, 3H), 1.24-1.35 (m, 2H), 1.39-1.52 (m, 2H), 1.70-2.07 (m, 11H), 2.12-2.37 (m, 5H), 2.44 (q, J=7.33 Hz, 2H), 2.49-2.73 (m, 6H), 3.83-3.94 (m, 1H), 3.98 (s, 3H), 4.59 (quin, J=8.15 Hz, 1H), 4.96 (dd, J=8.84, 3.54 Hz, 1H), 5.96 (d, J=8.08 Hz, 1H), 7.24 (dd, J=8.46, 1.89 Hz, 1H), 7.43 (d, J=1.77 Hz, 1H), 7.77 (s, 1H), 7.94 (s, 1H), 8.31 (s, 1H), 8.49 (d, J=8.34 Hz, 1H). MS (ES) [M+H] found 653, m.p.: 170-173° C.

### Example 4

### 4-(R)-7-Cyano-5-cyclopentyl-6-ethyl-5,6-dihydroimidazo[1,5-f]pteridin-3-ylamino)-N-(4-(4-isopropylpiperazin-1-yl)cyclohexyl)-3-methoxybenzamide

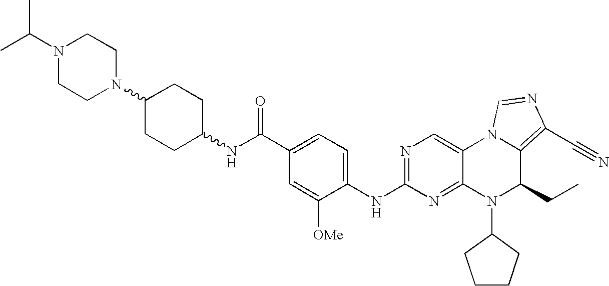

The title compound was prepared using a method analogous to Example 1 starting from (R)-4-(7-cyano-5-cyclopentyl-6-ethyl-5,6-dihydroimidazo[1,5-f]pteridin-3-ylamino)-3-methoxybenzoic acid and 4-(4-isopropylpiperazin-1-yl)cyclohexanamine. The final compounds were separated using normal phase chromatography to give the product in two fractions. The faster fraction (minor): ¹H [M+H] (400 MHz, CDCl₃) δ ppm 0.90 (t, 3H) 1.10 (d, J=6.32 Hz, 6H) 1.53-2.12 (m, 16H) 2.12-2.40 (m, 2H) 2.41-2.99 (m, 10H) 4.00 (s, 3H) 4.28 (br. s., 1H) 4.62 (dq, J=8.08, 7.92 Hz, 1H) 4.96 (dd, J=8.72, 3.41 Hz, 1H) 6.42 (br. s., 1H) 7.31 (d, J=8.08 Hz, 1H) 7.48 (s, 1H) 7.79 (s, 1H) 7.97 (s, 1H) 8.33 (s, 1H) 8.50 (d, J=8.34 Hz, 1H). MS (ES) [M+H] found 667. The slower fraction (major): ¹H [M+H] (400 MHz, CDCl₃) δ ppm 0.90 (t, 3H) 1.10 (d, J=6.32 Hz, 6H) 1.53-2.12 (m, 16H) 2.12-2.40 (m, 2H) 2.41-2.99 (m, 10H) 4.00 (s, 4H) 4.62 (dq, J=8.08, 7.92 Hz, 1H) 4.96 (dd, J=8.72, 3.41 Hz, 1H) 6.42 (br. s., 1H) 7.31 (d, J=8.08 Hz, 1H) 7.48 (s, 1H) 7.79 (s, 1H) 7.97 (s, 1H) 8.33 (s, 1H) 8.50 (d, J=8.34 Hz, 1H). MS (ES) [M+H] found 667. m.p.: 216-220° C.

### Example 5

### 4-(R)-7-Cyano-5-cyclopentyl-6-ethyl-5,6-dihydroimidazo[1,5-f]pteridin-3-ylamino)-N-((1r,4R)-4-(4-(cyclopropylmethyl)piperazin-1-yl)cyclohexyl)-3-methoxybenzamide

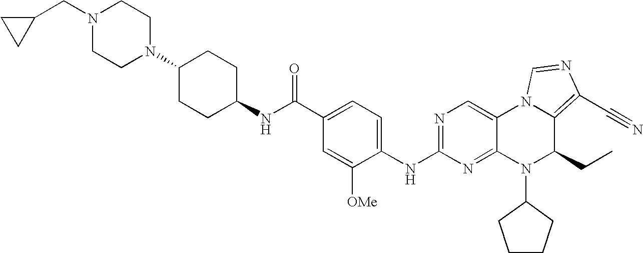

The title compound was prepared using a method analogous to Example 1 starting from (R)-4-(7-cyano-5-cyclopentyl-6-ethyl-5,6-dihydroimidazo[1,5-f]pteridin-3-ylamino)-3-methoxybenzoic acid and 4-(4-(cyclopropylmethyl)piperazin-1-yl)cyclohexanamine. ¹H NMR (400 MHz, CDCl₃) δ ppm 0.05-0.18 (m, 2H), 0.48-0.58 (m, 2H), 0.87 (t, J=7.45 Hz, 3H), 1.28-1.36 (m, 2H), 1.39-1.55 (m, 2H), 1.70-2.04 (m, 11H), 2.14-2.39 (m, 7H), 2.67 (br. s., 7H), 3.90-3.99 (m, 4H), 4.51-4.67 (m, 1H), 4.96 (dd, J=8.84, 3.54 Hz, 1H), 5.92 (d, J=8.08 Hz, 1H), 7.24 (dd, J=8.46, 1.89 Hz, 1H), 7.44 (d, J=2.02 Hz, 1H), 7.78 (s, 1H), 7.94 (s, 1H), 8.31 (s, 1H), 8.49 (d, J=8.59 Hz, 1H), MS (ES) [M+H] found 679, m.p.: 186-190° C.

### Example 6

### 4-(R)-7-Cyano-5-cyclopentyl-6-ethyl-5,6-dihydroimidazo[1,5-f]pteridin-3-ylamino)-2-fluoro-5-methoxy-N-(4-(4-methylpiperazin-1-yl)cyclohexyl)benzamide

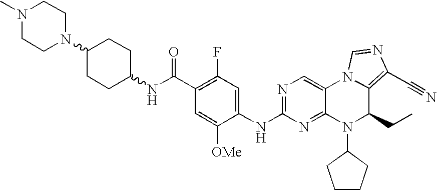

The title compound was prepared using a method analogous to Example 1 starting from (R)-4-(5-cyclopentyl-7-cyano-6-ethyl-5,6-dihydroimidazo[1,5-f]pteridin-3-ylamino)-2-fluoro-5-methoxybenzoic acid and 4-(4-methylpiperazin-1-yl)cyclohexanamine. The final compounds were separated using normal phase chromatography to give the product in two fractions. The faster fraction (minor): MS (ES) [M+H] found 657. The slower fraction (major): ¹H NMR (400 MHz, CDCl₃) δ ppm 0.84 (t, J=7.45 Hz, 3H), 1.22-1.33 (m, 2H), 1.36-1.49 (m, 2H), 1.72-1.94 (m, 11H), 2.15-2.34 (m, 7H), 2.47 (br. s., 3H), 2.61 (br. s., 4H), 3.85-3.98 (m, 4H), 4.53-4.64 (m, 1H), 4.97 (dd, J=8.72, 3.41 Hz, 1H), 6.56-6.62 (m, 1H), 7.54 (d, J=7.07 Hz, 1H), 7.82 (s, 1H), 7.97 (s, 1H), 8.33-8.39 (m, 2H). MS (ES) [M+H] found 657. m.p.: >205° C.

### Example 7

### 4-(R)-7-Cyano-5-cyclopentyl-6-ethyl-5,6-dihydroimidazo[1,5-f]pteridin-3-ylamino)-N-(4-(4-ethylpiperazin-1-yl)cyclohexyl)-2-fluoro-5-methoxybenzamide

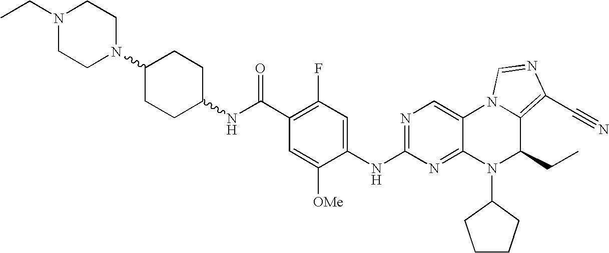

The title compound was prepared using a method analogous to Example 1 starting from (R)-4-(5-cyclopentyl-7-cyano-6-ethyl-5,6-dihydroimidazo[1,5-f]pteridin-3-ylamino)-2-fluoro-5-methoxybenzoic acid and 4-(4-ethylpiperazin-1-yl)cyclohexanamine. The final compounds were separated using normal phase chromatography to give the product in two fractions. The faster fraction (minor): MS (ES) [M+H] found 671. The slower fraction (major): ¹H NMR (400 MHz, CDCl₃) δ ppm 0.87 (t, J=7.45 Hz, 3H), 1.12 (t, J=7.33 Hz, 3H), 1.23-1.36 (m, 2H), 1.39-1.52 (m, 2H), 1.72-2.08 (m, 11H), 2.14-2.28 (m, 3H), 2.36 (tt, J=11.40, 3.13 Hz, 1H), 2.50 (q, J=7.16 Hz, 2H), 2.55-2.75 (m, 7H), 3.89-4.00 (m, 4H), 4.53-4.67 (m, 1H), 4.99 (dd, J=8.72, 3.41 Hz, 1H), 6.62-6.59 (m, 1H), 7.56 (d, J=7.33 Hz, 1H), 7.85 (s, 1H), 7.96 (s, 1H), 8.30-8.45 (m, 2H). MS (ES) [M+H] found 671.

### Example 8

### 4-(R)-7-Cyano-5-cyclopentyl-6-ethyl-5,6-dihydroimidazo[1,5-f]pteridin-3-ylamino)-2-fluoro-N-(4-(4-isobutylpiperazin-1-yl)cyclohexyl)-5-methoxybenzamide

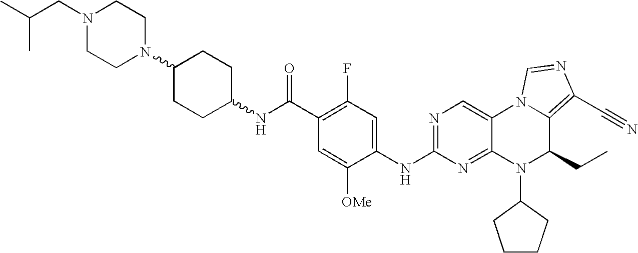

The title compound was prepared using a method analogous to Example 1 starting from (R)-4-(5-cyclopentyl-7-cyano-6-ethyl-5,6-dihydroimidazo[1,5-f]pteridin-3-ylamino)-2-fluoro-5-methoxybenzoic acid and 4-(4-isobutylpiperazin-1-yl)cyclohexanamine hydrochloride. ¹H NMR (400 MHz, DMSO-d₆) δ ppm 0.75 (t, J=7.33 Hz, 3H) 0.80-0.92 (m, 6H) 1.18-1.46 (m, 4H) 1.62 (br. s., 2H) 1.66-1.87 (m, 8H) 1.91-2.02 (m, 10H) 2.08 (br. s., 1H) 2.19 (br. s., 1H) 2.25-2.41 (m, 4H) 3.68 (m, 1H) 3.91 (s, 3H) 4.48 (quin, J=8.59 Hz, 1H) 5.21 (dd, J=8.21, 3.66 Hz, 1H) 7.19 (d, J=6.82 Hz, 1H) 7.83 (dd, J=7.71, 3.66 Hz, 1H) 8.03 (s, 1H) 8.24 (d, J=13.14 Hz, 1H) 8.65 (s, 1H) 8.75 (s, 1H). MS (ES) [M+H] found 699, m.p.: 155-158° C.

### Example 9

### 4-(R)-7-Cyano-5-isopropyl-6-ethyl-5,6-dihydroimidazo[1,5-f]pteridin-3-ylamino)-3-methoxy-N-(4-(4-methylpiperazin-1-yl)cyclohexyl)benzamide

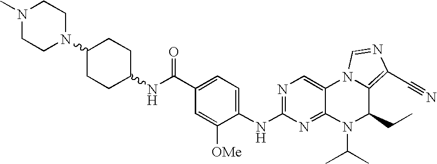

The title compound was prepared using a method analogous to Example 1 starting from (R)-4-(7-cyano-6-ethyl-5-isopropyl-5,6-dihydroimidazo[1,5-f]pteridin-3-ylamino)-3-methoxybenzoic acid and 4-(4-methylpiperazin-1-yl)cyclohexanamine. ¹H NMR (400 MHz, CDCl₃) δ ppm 0.86 (t, J=7.45 Hz, 3H), 1.27-1.35 (m, 2H), 1.39-1.56 (m, 8H), 1.82-2.03 (m, 6H), 2.18-2.21 (m, 2H), 2.28-2.65 (m, 9H), 3.87-3.96 (m, 1H), 3.99 (s, 3H), 4.72-4.86 (m, 1H), 5.06 (dd, J=8.34, 3.28 Hz, 1H), 5.90 (d, J=7.83 Hz, 1H), 7.25 (dd, J=8.46, 1.78 Hz, 1H), 7.45 (d, J=1.77 Hz, 1H), 7.81 (s, 1H), 7.94 (s, 1H), 8.32 (s, 1H), 8.51 (d, J=8.34 Hz, 1H). MS (ES) [M+H] found 613. m.p.: 195-198° C.

### Example 10

### 4-(R)-7-Cyano-6-ethyl-5-isopropyl-5,6-dihydroimidazo[1,5-f]pteridin-3-ylamino)-N-(4-(4-isopropylpiperazin-1-yl)cyclohexyl)-3-methoxybenzamide

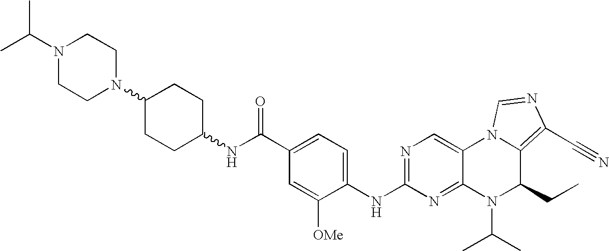

The title compound was prepared using a method analogous to Example 1 starting from (R)-4-(7-cyano-5-isopropyl-6-ethyl-5,6-dihydroimidazo[1,5-f]pteridin-3-ylamino)-3-methoxybenzoic acid and 4-(4-isopropylpiperazin-1-yl)cyclohexanamine. The final compounds were separated using normal phase chromatography to give the product in two fractions. The faster fraction (minor): ¹H [M+H] (400 MHz, CDCl₃) δ ppm 0.86 (t, J=7.45 Hz, 3H) 1.07 (d, J=6.57 Hz, 6H) 1.44 (d, J=6.82 Hz, 3H) 1.55 (d, J=6.82 Hz, 6H) 1.62-1.76 (m, 2H) 1.75-2.00 (m, 6H) 2.18 (t, J=9.09 Hz, 2H) 2.63 (ddd, J=12.88, 6.32, 6.06 Hz, 8H) 3.99 (s, 3H) 4.27 (d, J=3.79 Hz, 1H) 4.83 (quin, J=6.88 Hz, 1H) 5.07 (dd, J=8.34, 3.54 Hz, 1H) 6.25 (d, J=7.58 Hz, 1H) 7.26 (dd, J=8.34, 1.77 Hz, 1H) 7.83 (s, 1H) 7.95 (s, 1H) 8.33 (s, 1H) 8.51 (d, J=8.34 Hz, 1H). MS (ES) [M+H] found 641. The slower fraction(major): ¹H [M+H] (400 MHz, CDCl₃) δ ppm 0.86 (t, J=7.45 Hz, 3H) 1.07 (d, J=6.57 Hz, 6H) 1.44 (d, J=6.82 Hz, 3H) 1.55 (d, J=6.82 Hz, 6H) 1.62-1.76 (m, 2H) 1.75-2.00 (m, 6H) 2.18 (t, J=9.09 Hz, 2H) 2.63 (ddd, J=12.88, 6.32, 6.06 Hz, 8H) 3.99 (s, 4H) 4.83 (quin, J=6.88 Hz, 1H) 5.07 (dd, J=8.34, 3.54 Hz, 1H) 6.25 (d, J=7.58 Hz, 1H) 7.26 (dd, J=8.34, 1.77 Hz, 1H) 7.83 (s, 1H) 7.95 (s, 1H) 8.33 (s, 1H) 8.51 (d, J=8.34 Hz, 1H). MS (ES) [M+H] found 641.

### Example 11

### 4-(R)-7-Cyano-6-ethyl-5-isopropyl-5,6-dihydroimidazo[1,5-f]pteridin-3-ylamino)-N-((1r,4R)-4-(4-(cyclopropylmethyl)piperazin-1-yl)cyclohexyl)-3-methoxybenzamide

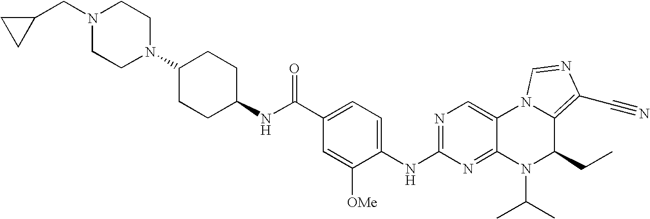

The title compound was prepared using a method analogous to Example 1 starting from (R)-4-(7-cyano-5-isopropyl-6-ethyl-5,6-dihydroimidazo[1,5-f]pteridin-3-ylamino)-3-methoxybenzoic acid and (1r,4r)-4-(4-(cyclopropylmethyl)piperazin-1-yl)cyclohexanamine hydrochloride. ¹H NMR (400 MHz, CHLOROFORM-d) δ ppm 0.08-0.15 (m, 2H) 0.48-0.57 (m, 2H) 0.66-0.90 (m, 4H) 1.17-1.36 (m, 3H) 1.39-1.55 (m, 8H) 1.78-2.08 (m, 4H) 2.19 (d, J=12.38 Hz, 2H) 2.24-2.38 (m, 3H) 2.38-2.97 (m, 8H) 3.84-4.04 (m, 4H) 4.73-4.89 (m, 1H) 5.06 (dd, J=8.34, 3.28 Hz, 1H) 5.89 (d, J=8.08 Hz, 1H) 7.22-7.26 (m, 1H) 7.45 (d, J=1.52 Hz, 1H) 7.81 (s, 1H) 7.94 (s, 1H) 8.32 (s, 1H) 8.51 (d, J=8.34 Hz, 1H). MS (ES) [M+H] found 653. m.p.: 198-204° C.

### Example 12

### 4-(R)-7-Cyano-6-ethyl-5-isopropyl-5,6-dihydroimidazo[1,5-f]pteridin-3-ylamino)-N-(4-(4-isobutylpiperazin-1-yl)cyclohexyl)-3-methoxybenzamide

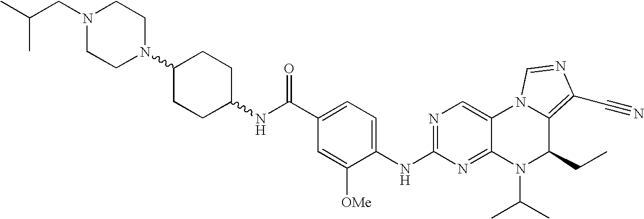

The title compound was prepared using a method analogous to Example 1 starting from (R)-4-(7-cyano-5-isopropyl-6-ethyl-5,6-dihydroimidazo[1,5-f]pteridin-3-ylamino)-3-methoxybenzoic acid and 4-(4-isobutylpiperazin-1-yl)cyclohexanamine hydrochloride. The final compounds were separated using normal phase chromatography to give the product in two fractions. The faster fraction (minor): ¹H NMR (400 MHz, DMSO-d₆) δ ppm 0.73 (br. s., 3H) 0.78-0.94 (m, 6H) 1.09 (br. s., 1H) 1.21-1.50 (m, 11H) 1.81 (br. s., 7H) 1.98 (br. s., 3H) 2.30 (m, 6H) 3.71 (br. s., 1H) 3.93 (s, 3H) 4.61 (m, 1H) 5.23 (m, 1H) 7.49 (m, 2H) 7.97 (br. s., 1H) 8.07 (m, 1H) 8.32 (br. s., 1H) 8.63 (br. s., 1H) 8.71 (br. s., 1H). MS (ES) [M+H] found 655. The slower fraction (major): ¹H NMR (400 MHz, DMSO-d₆) δ ppm 0.73 (t, J=7.33 Hz, 3H) 0.84 (d, J=6.57 Hz, 6H) 1.30-1.59 (m, 10H) 1.61-1.85 (m, 5H) 1.91 (br. s., 3H) 1.94-2.05 (m, 2H) 2.08 (br. s., 2H) 2.21-2.48 (m, 6H) 3.92 (m, 1H) 3.94 (s, 3H) 4.61 (dt, J=13.58, 6.73 Hz, 1H) 5.23 (dd, J=6.95, 4.67 Hz, 1H) 7.42-7.64 (m, 2H) 7.97 (s, 1H) 8.07 (d, J=7.33 Hz, 1H) 8.32 (d, J=8.34 Hz, 1H) 8.64 (s, 1H) 8.71 (s, 1H). MS (ES) [M+H] found 655. m.p.: 165-170° C.

### Example 13

### 4-(R)-7-Cyano-6-ethyl-5-isopropyl-5,6-dihydroimidazo[1,5-f]pteridin-3-ylamino)-2-fluoro-5-methoxy-N-(4-(4-methylpiperazin-1-yl)cyclohexyl)benzamide

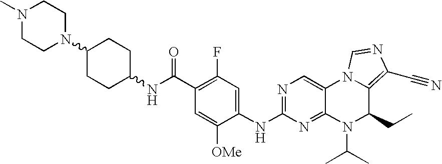

The title compound was prepared using a method analogous to Example 1 starting from (R)-4-(7-cyano-5-isopropyl-6-ethyl-5,6-dihydroimidazo[1,5-f]pteridin-3-ylamino)-2-fluoro-5-methoxybenzoic acid and 4-(4-methylpiperazin-1-yl)cyclohexanamine. The final compounds were separated using normal phase chromatography to give the product in two fractions. The faster fraction (minor): ¹H NMR (400 MHz, CDCl₃) δ ppm 0.86 (t, J=7.45 Hz, 3H) 1.10 (d, J=6.57 Hz, 6H) 1.45 (d, J=6.82 Hz, 3H) 1.56 (d, J=6.82 Hz, 4H) 1.67 (t, J=12.51 Hz, 5H) 1.75-1.88 (m, 3H) 1.94 (dddd, J=10.55, 7.20, 3.73, 3.54 Hz, 3H) 2.49-2.86 (m, 8H) 3.97 (s, 3H) 4.28 (d, J=3.03 Hz, 1H) 4.81 (quin, J=6.88 Hz, 1H) 5.09 (dd, J=8.21, 3.41 Hz, 1H) 6.94 (dd, J=15.41, 7.33 Hz, 1H) 7.58 (d, J=7.07 Hz, 1H) 7.87 (s, 1H) 7.96 (s, 1H) 8.34 (s, 1H) 8.38-8.50 (m, 1H) [M+H] found 631. The slower fraction (major): ¹H NMR (400 MHz, CDCl₃) δ ppm 0.86 (t, J=7.45 Hz, 3H) 1.10 (d, J=6.57 Hz, 6H) 1.45 (d, J=6.82 Hz, 3H) 1.56 (d, J=6.82 Hz, 4H) 1.67 (t, J=12.51 Hz, 5H) 1.75-1.88 (m, 3H) 1.94 (dddd, J=10.55, 7.20, 3.73, 3.54 Hz, 3H) 2.49-2.86 (m, 8H) 3.97 (s, 4H) 4.81 (quin, J=6.88 Hz, 1H) 5.09 (dd, J=8.21, 3.41 Hz, 1H) 6.94 (dd, J=15.41, 7.33 Hz, 1H) 7.58 (d, J=7.07 Hz, 1H) 7.87 (s, 1H) 7.96 (s, 1H) 8.34 (s, 1H) 8.38-8.50 (m, 1H). MS (ES) [M+H] found 631.

### Example 14

### 4-(R)-7-Cyano-6-ethyl-5-isopropyl-5,6-dihydroimidazo[1,5-f]pteridin-3-ylamino)-N-(4-(4-ethylpiperazin-1-yl)cyclohexyl)-2-fluoro-5-methoxybenzamide

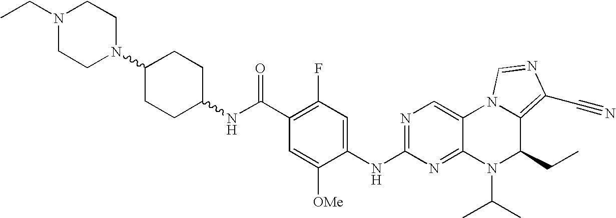

The title compound was prepared using a method analogous to Example 1 starting from (R)-4-(5-cyclopentyl-7-cyano-6-ethyl-5,6-dihydroimidazo[1,5-f]pteridin-3-ylamino)-2-fluoro-5-methoxybenzoic acid and 4-(4-ethylpiperazin-1-yl)cyclohexanamine. The final compounds were separated using normal phase chromatography to give the product in two fractions. The faster fraction (minor): ¹H NMR (400 MHz, CDCl₃) δ ppm 0.86 (t, J=7.33 Hz, 3H) 1.11 (t, J=7.20 Hz, 3H) 1.21-1.36 (m, 2H) 1.45 (d, J=6.82 Hz, 3H) 1.56 (d, J=6.82 Hz, 3H) 1.67 (t, J=12.38 Hz, 2H) 1.74-1.88 (m, 4H) 1.94 (dddd, J=10.45, 7.23, 3.79, 3.54 Hz, 3H) 2.27 (t, J=9.22 Hz, 3H) 2.46 (q, J=7.16 Hz, 3H) 2.65 (br. s., 4H) 3.97 (s, 4H) 4.81 (quin, J=6.82 Hz, 1H) 5.09 (dd, J=8.21, 3.41 Hz, 1H) 6.94 (dd, J=15.41, 7.33 Hz, 1H) 7.58 (d, J=7.07 Hz, 1H) 7.87 (s, 1H) 7.96 (s, 1H) 8.34 (s, 1H) 8.44 (d, J=15.16 Hz, 1H). MS (ES) [M+H] found 645. The slower fraction(major): ¹H NMR (400 MHz, CDCl₃) δ ppm 0.86 (t, J=7.33 Hz, 3H) 1.11 (t, J=7.20 Hz, 3H) 1.21-1.36 (m, 2H) 1.45 (d, J=6.82 Hz, 3H) 1.56 (d, J=6.82 Hz, 3H) 1.67 (t, J=12.38 Hz, 2H) 1.74-1.88 (m, 4H) 1.94 (dddd, J=10.45, 7.23, 3.79, 3.54 Hz, 3H) 2.27 (t, J=9.22 Hz, 3H) 2.46 (q, J=7.16 Hz, 3H) 2.65 (br. s., 4H) 3.97 (s, 3H) 4.27 (d, J=3.28 Hz, 1H) 4.81 (quin, J=6.82 Hz, 1H) 5.09 (dd, J=8.21, 3.41 Hz, 1H) 6.94 (dd, J=15.41, 7.33 Hz, 1H) 7.58 (d, J=7.07 Hz, 1H) 7.87 (s, 1H) 7.96 (s, 1H) 8.34 (s, 1H) 8.44 (d, J=15.16 Hz, 1H). MS (ES) [M+H] found 645.

### Example 15

### 4-(R)-7-Cyano-6-ethyl-5-isopropyl-5,6-dihydroimidazo[1,5-f]pteridin-3-ylamino)-2-fluoro-N-(4-(4-isopropylpiperazin-1-yl)cyclohexyl)-5-methoxybenzamide

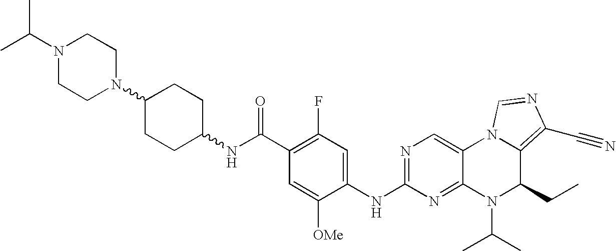

The title compound was prepared using a method analogous to Example 1 starting from (R)-4-(5-cyclopentyl-7-cyano-6-ethyl-5,6-dihydroimidazo[1,5-f]pteridin-3-ylamino)-2-fluoro-5-methoxybenzoic acid and 4-(4-isopropylpiperazin-1-yl)cyclohexanamine. The final compounds were separated using normal phase chromatography to give the product in two fractions. The faster fraction (minor): ¹H NMR (400 MHz, CDCl₃) δ ppm 0.86 (t, J=7.45 Hz, 3H) 1.10 (d, J=6.57 Hz, 6H) 1.45 (d, J=6.82 Hz, 3H) 1.56 (d, J=6.82 Hz, 4H) 1.67 (t, J=12.51 Hz, 5H) 1.75-1.88 (m, 3H) 1.94 (dddd, J=10.55, 7.20, 3.73, 3.54 Hz, 3H) 2.49-2.86 (m, 8H) 3.97 (s, 3H) 4.28 (d, J=3.03 Hz, 1H) 4.81 (quin, J=6.88 Hz, 1H) 5.09 (dd, J=8.21, 3.41 Hz, 1H) 6.94 (dd, J=15.41, 7.33 Hz, 1H) 7.58 (d, J=7.07 Hz, 1H) 7.87 (s, 1H) 7.96 (s, 1H) 8.34 (s, 1H) 8.38-8.50 (m, 1H). MS (ES) [M+H] found 659. The slower fraction (major): ¹H NMR (400 MHz, CDCl₃) δ ppm 0.86 (t, J=7.45 Hz, 3H) 1.10 (d, J=6.57 Hz, 6H) 1.45 (d, J=6.82 Hz, 3H) 1.56 (d, J=6.82 Hz, 4H) 1.67 (t, J=12.51 Hz, 5H) 1.75-1.88 (m, 3H) 1.94 (dddd, J=10.55, 7.20, 3.73, 3.54 Hz, 3H) 2.49-2.86 (m, 8H) 3.97 (s, 4H) 4.81 (quin, J=6.88 Hz, 1H) 5.09 (dd, J=8.21, 3.41 Hz, 1H) 6.94 (dd, J=15.41, 7.33 Hz, 1H) 7.58 (d, J=7.07 Hz, 1H) 7.87 (s, 1H) 7.96 (s, 1H) 8.34 (s, 1H) 8.38-8.50 (m, 1H). MS (ES) [M+H] found 659.

### Example 16

### 4-(R)-7-Cyano-6-ethyl-5-isopropyl-5,6-dihydroimidazo[1,5-f]pteridin-3-ylamino)-2-fluoro-5-methoxy-N-(4-(4-propylpiperazin-1-yl)cyclohexyl)benzamide

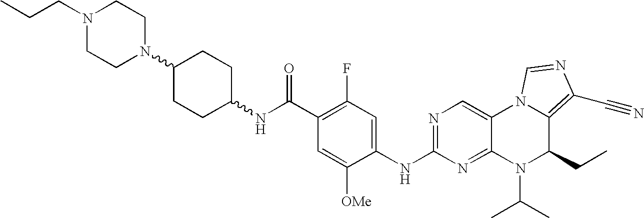

The title compound was prepared using a method analogous to Example 1 starting from (R)-4-(5-cyclopentyl-7-cyano-6-ethyl-5,6-dihydroimidazo[1,5-f]pteridin-3-ylamino)-2-fluoro-5-methoxybenzoic acid and 4-(4-propylpiperazin-1-yl)cyclohexanamine. The final compounds were separated using normal phase chromatography to give the product in two fractions. The faster fraction (minor): ¹H [M+H] (400 MHz, CDCl₃) δ ppm 0.89 (dt, J=19.01, 7.42 Hz, 6H) 1.32-1.61 (m, 10H) 1.60-2.06 (m, 10H) 2.15-2.37 (m, 3H) 2.38-2.92 (m, 6H) 3.97 (s, 3H) 4.27 (d, J=3.28 Hz, 1H) 4.81 (quin, J=6.82 Hz, 1H) 5.09 (dd, J=8.21, 3.41 Hz, 1H) 6.94 (dd, J=15.54, 7.45 Hz, 1H) 7.58 (d, J=7.07 Hz, 1H) 7.87 (s, 1H) 7.95 (d, J=2.02 Hz, 1H) 8.34 (s, 1H) 8.38-8.53 (m, 1H). MS (ES) [M+H] found 659. The slower fraction (major): ¹H [M+H] (400 MHz, CDCl₃) δ ppm 0.89 (dt, J=19.01, 7.42 Hz, 6H) 1.32-1.61 (m, 10H) 1.60-2.06 (m, 10H) 2.15-2.37 (m, 3H) 2.38-2.92 (m, 6H) 3.97 (s, 4H) 4.81 (quin, J=6.82 Hz, 1H) 5.09 (dd, J=8.21, 3.41 Hz, 1H) 6.94 (dd, J=15.54, 7.45 Hz, 1H) 7.58 (d, J=7.07 Hz, 1H) 7.87 (s, 1H) 7.95 (d, J=2.02 Hz, 1H) 8.34 (s, 1H) 8.38-8.53 (m, 1H). MS (ES) [M+H] found 659.

### Example 17

### 4-(R)-7-Cyano-5-cyclopentyl-6-ethyl-5,6-dihydroimidazo[1,5-f]pteridin-3-ylamino)-2-fluoro-5-methoxy-N-((1r,4S)-4-(4-methylpiperazin-1-yl)cyclohexyl)benzamide

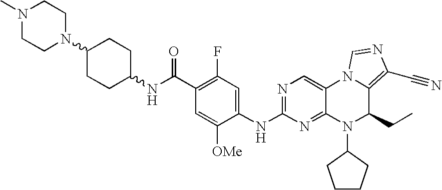

The title compound was prepared using a method analogous to Example 1 starting from (R)-4-(5-cyclopentyl-7-cyano-6-ethyl-5,6-dihydroimidazo[1,5-f]pteridin-3-ylamino)-2-fluoro-5-methoxybenzoic acid and 4-(4-methylpiperazin-1-yl)cyclohexanamine. ¹H NMR (400 MHz, CHLOROFORM-d) δ ppm 0.84 (t, J=7.45 Hz, 3H), 1.22-1.33 (m, 2H), 1.36-1.49 (m, 2H), 1.72-1.94 (m, 11H), 2.15-2.34 (m, 7H), 2.47 (br. s., 3H), 2.61 (br. s., 4H), 3.85-3.98 (m, 4H), 4.53-4.64 (m, 1H), 4.97 (dd, J=8.72, 3.41 Hz, 1H), 6.56-6.62 (m, 1H), 7.54 (d, J=7.07 Hz, 1H), 7.82 (s, 1H), 7.97 (s, 1H), 8.33-8.39 (m, 2H). MS (ES) [M+H] found 657.

### Example 18

### 4-(R)-7-Cyano-6-ethyl-5-isopropyl-5,6-dihydroimidazo[1,5-f]pteridin-3-ylamino)-2-fluoro-N-(4-(4-isobutylpiperazin-1-yl)cyclohexyl)-5-methoxybenzamide

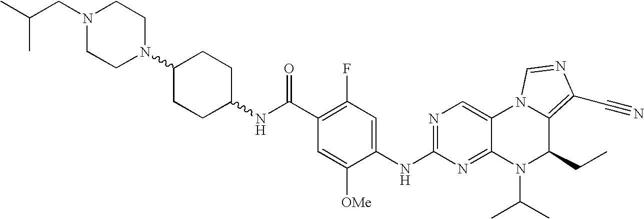

The title compound was prepared using a method analogous to Example 1 starting from (R)-4-(5-cyclopentyl-7-cyano-6-ethyl-5,6-dihydroimidazo[1,5-f]pteridin-3-ylamino)-2-fluoro-5-methoxybenzoic acid and 4-(4-isobutylpiperazin-1-yl)cyclohexanamine hydrochloride. The final compounds were separated using normal phase chromatography to give the product in two fractions. The slower fraction (major): ¹H NMR (400 MHz, DMSO-d₆) δ ppm 0.75 (t, J=7.33 Hz, 3H) 1.27-1.46 (m, 1H) 1.46-1.67 (m, 4H) 1.67-1.86 (m, 5H) 1.86-1.96 (m, 3H) 2.01-2.17 (m, 1H) 2.42 (m, 2H) 2.63-2.82 (m, 1H) 2.92 (dd, J=11.75, 3.16 Hz, 1H) 3.83 (dt, J=8.21, 3.98 Hz, 1H) 3.91 (s, 3H) 4.34-4.63 (m, 1H) 5.20 (dd, J=8.21, 3.66 Hz, 1H) 7.24 (d, J=6.82 Hz, 1H) 7.79 (dd, J=7.83, 5.31 Hz, 1H) 8.04 (s, 1H) 8.25 (d, J=13.64 Hz, 1H) 8.65 (s, 1H) 8.75 (s, 1H). MS (ES) [M+H] found 673, m.p.: 219-222° C. The faster fraction (minor): MS (ES) [M+H] found 673.

### Example 19

### (R)-4-(7-Cyano-6-ethyl-5-isopropyl-5,6-dihydroimidazo[1,5-f]pteridin-3-ylamino)-N-(1′-ethyl-1,4′-bipiperidin-4-yl)-3-methoxybenzamide

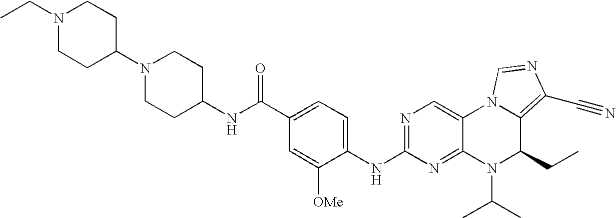

The title compound was prepared using a method analogous to Example 1 starting from (R)-4-(5-isopropyl-7-cyano-6-ethyl-5,6-dihydroimidazo[1,5-f]pteridin-3-ylamino)-5-methoxybenzoic acid and 1′-ethyl-1,4′-bipiperidin-4-amine hydrochloride. The title compound was purified by reverse chromatography to give 55 mg (37%). ¹H NMR (400 MHz, DMSO-d₆) δ ppm 0.73 (t, J=7.33 Hz, 3H) 0.99 (t, J=6.95 Hz, 3H) 1.41 (dd, 6H) 1.48-1.60 (m, 2H) 1.62-1.95 (m, 7H) 2.10-2.39 (m, 5H) 2.90 (br. s., 4H) 3.75 (br. s., 1H) 3.93 (s, 3H) 4.54-4.68 (m, 1H) 5.23 (dd, J=6.95, 4.67 Hz, 1H) 7.49 (s, 1H) 7.52 (dd, 1H) 7.98 (s, 1H) 8.11 (d, J=8.08 Hz, 1H) 8.33 (d, J=8.59 Hz, 1H) 8.64 (s, 1H) 8.71 (s, 1H). MS (ES) [M+H] found 627.

### Example 20

### (R)-4-(7-Cyano-6-ethyl-5-isopropyl-5,6-dihydroimidazo[1,5-f]pteridin-3-ylamino)-N-(1′-ethyl-1,4′-bipiperidin-4-yl)-2-fluoro-5-methoxybenzamide

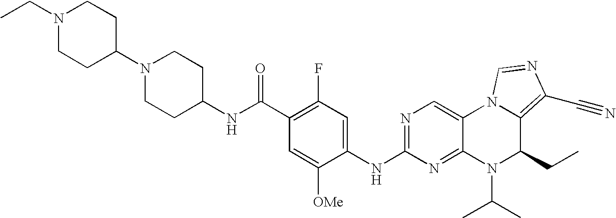

The title compound was prepared using a method analogous to Example 1 starting from (R)-4-(5-isopropyl-7-cyano-6-ethyl-5,6-dihydroimidazo[1,5-f]pteridin-3-ylamino)-2-fluoro-5-methoxybenzoic acid and 1′-ethyl-1,4′-bipiperidin-4-amine. The title compound was purified by reverse phase chromatography to give 183 mg (43%). ¹H NMR (400 MHz, DMSO-d₆) δ ppm 0.73 (t, J=7.33 Hz, 3H) 1.17 (t, J=7.20 Hz, 3H) 1.20-1.31 (m, 1H) 1.43 (dd, J=19.71, 6.82 Hz, 6H) 1.53-2.21 (m, 8H) 2.57-3.21 (m, 6H) 3.91 (s, 3H) 4.61 (quin, J=6.82 Hz, 1H) 5.26 (dd, J=6.82, 4.80 Hz, 1H) 7.19 (d, J=6.57 Hz, 1H) 8.06 (s, 2H) 8.31 (d, J=13.39 Hz, 1H) 8.65 (s, 1H) 8.75 (s, 1H). MS (ES) [M+H] found 645.

### Example 21

### (R)-4-(7-Cyano-6-ethyl-5-isopropyl-5,6-dihydroimidazo[1,5-f]pteridin-3-ylamino)-N-(1′-(cyclopropylmethyl)-1,4′-bipiperidin-4-yl)-3-methoxybenzamide

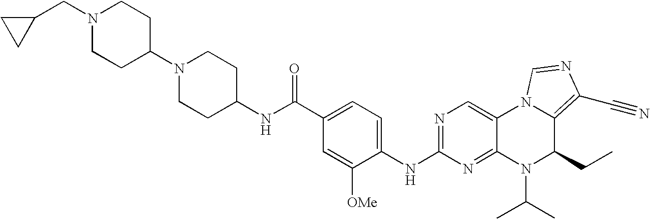

The title compound was prepared using a method analogous to Example 1 starting from (R)-4-(7-cyano-6-ethyl-5-isopropyl-5,6-dihydroimidazo[1,5-f]pteridin-3-ylamino)-3-methoxybenzoic acid and 1′-(cyclopropylmethyl)-1,4′-bipiperidin-4-amine hydrochloride. The title compound was purified by reverse phase chromatography. ¹H NMR (400 MHz, DMSO-d₆) δ ppm 0.08 (d, J=4.29 Hz, 2H) 0.33-0.52 (m, 2H) 0.73 (t, J=7.33 Hz, 3H) 0.79-0.93 (m, 1H) 1.41 (dd, 8H) 1.53 (br. s., 3H) 1.65-1.84 (m, 6H) 1.99 (s, 2H) 2.23 (br. s., 5H) 2.85-3.09 (m, 4H) 3.69-3.82 (m, 1H) 3.93 (s, 3H) 4.61 (quin, J=6.76 Hz, 1H) 5.23 (dd, J=6.95, 4.42 Hz, 1H) 7.49 (d, J=1.77 Hz, 1H) 7.51 (dd, 1H) 7.97 (s, 1H) 8.13 (d, J=7.58 Hz, 1H) 8.34 (d, J=8.59 Hz, 1H) 8.63 (s, 1H) 8.71 (s, 1H). MS (ES) [M+H] found 653.

### Example 22

### (R)-4-(7-Cyano-6-ethyl-5-isopropyl-5,6-dihydroimidazo[1,5-f]pteridin-3-ylamino)-N-(1′-(cyclopropylmethyl)-1,4′-bipiperidin-4-yl)-2-fluoro-5-methoxybenzamide

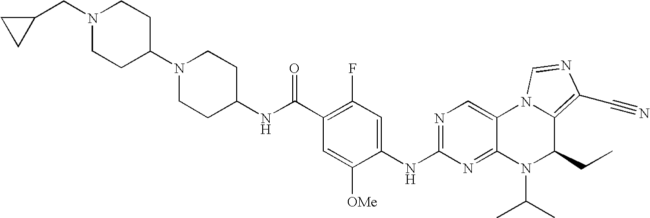

The title compound was prepared using a method analogous to Example 1 starting from (R)-4-(7-cyano-6-ethyl-5-isopropyl-5,6-dihydroimidazo[1,5-f]pteridin-3-ylamino)-2-fluoro-5-methoxybenzoic acid and 1′-(cyclopropylmethyl)-1,4′-bipiperidin-4-amine hydrochloride. The title compound was purified by reverse phase chromatography to give 283 mg (65%). ¹H NMR (400 MHz, DMSO-d₆) δ ppm 0.06 (d, J=5.56 Hz, 2H) 0.38-0.49 (m, 2H) 0.73 (t, J=7.33 Hz, 3H) 0.80 (br. s., 1H) 1.43 (dd, J=19.71, 6.82 Hz, 6H) 1.49-1.58 (m, 4H) 1.64-2.02 (m, 8H) 2.20 (br. s., 4H) 2.84 (br. s., 2H) 3.01 (br. s., 2H) 3.71 (d, J=11.12 Hz, 1H) 3.91 (s, 3H) 4.61 (quin, J=6.88 Hz, 1H) 5.25 (dd, J=6.95, 4.67 Hz, 1H) 7.19 (d, J=6.82 Hz, 1H) 7.86 (br. s., 1H) 8.04 (d, J=1.01 Hz, 1H) 8.30 (d, J=13.39 Hz, 1H) 8.64 (s, 1H) 8.75 (s, 1H). MS (ES) [M+H] found 671.

### Example 23

### (R)-4-(7-Cyano-6-ethyl-5-isopropyl-5,6-dihydroimidazo[1,5-f]pteridin-3-ylamino)-N-(1′-isobutyl-1,4′-bipiperidin-4-yl)-3-methoxybenzamide

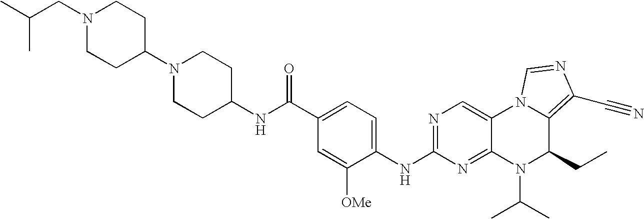

The title compound was prepared using a method analogous to Example 1 starting from (R)-4-(5-isopropyl-7-cyano-6-ethyl-5,6-dihydroimidazo[1,5-f]pteridin-3-ylamino)-5-methoxybenzoic acid and 1′-isobutyl-1,4′-bipiperidin-4-amine hydrochloride. The title compound was purified by reverse phase chromatography to give 48.8 mg (37%). ¹H NMR (400 MHz, DMSO-d₆) δ ppm 0.73 (t, J=7.33 Hz, 3H) 0.77-0.91 (m, 6H) 1.27-1.47 (m, 8H) 1.47-1.61 (m, 2H) 1.61-1.87 (m, 9H) 1.97 (d, J=7.33 Hz, 2H) 2.18 (t, J=10.86 Hz, 3H) 2.85 (dd, J=18.57, 11.49 Hz, 4H) 3.60-3.82 (m, 1H) 3.93 (s, 3H) 4.61 (quin, J=6.82 Hz, 1H) 5.23 (dd, J=6.82, 4.55 Hz, 1H) 7.47-7.59 (m, 2H) 7.97 (s, 1H) 8.11 (d, J=7.58 Hz, 1H) 8.33 (d, J=8.34 Hz, 1H) 8.64 (s, 1H) 8.71 (s, 1H). MS (ES) [M+H] found 655, m.p.: 165-170° C.

### Example 24

### (R)-4-(7-Cyano-6-ethyl-5-isopropyl-5,6-dihydroimidazo[1,5-f]pteridin-3-ylamino)-2-fluoro-N-(1′-isobutyl-1,4′-bipiperidin-4-yl)-5-methoxybenzamide

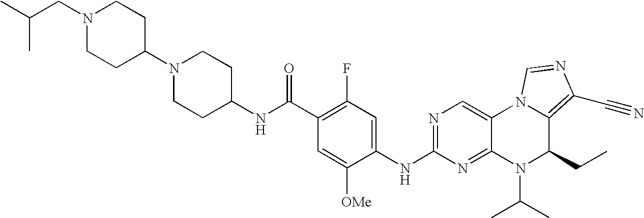

The title compound was prepared using a method analogous to Example 1 starting from (R)-4-(7-cyano-6-ethyl-5-isopropyl-5,6-dihydroimidazo[1,5-f]pteridin-3-ylamino)-2-fluoro-5-methoxybenzoic acid and 1′-isobutyl-1,4′-bipiperidin-4-amine hydrochloride. The title compound was purified by reverse phase chromatography to give 56 mg (42%). ¹H NMR (400 MHz, DMSO-d₆) δ ppm 0.73 (t, J=7.20 Hz, 3H) 0.83 (d, J=6.32 Hz, 6H) 1.30-1.55 (m, 10H) 1.57-1.75 (m, 4H) 1.78 (br. s., 5H) 1.97 (d, J=7.33 Hz, 2H) 2.19 (br. s., 3H) 2.81 (br. s., 4H) 3.71 (br. s., 1H) 3.91 (s, 3H) 4.48-4.73 (m, 1H) 5.25 (t, J=5.18 Hz, 1H) 7.19 (d, J=6.57 Hz, 1H) 7.77-7.92 (m, 1H) 8.03 (s, 1H) 8.30 (d, J=13.39 Hz, 1H) 8.65 (s, 1H) 8.75 (s, 1H). MS (ES) [M+H] found 673.

### Example 25

### 4-(R)-7-Cyano-5-cyclopentyl-6-ethyl-5,6-dihydroimidazo[1,5-f]pteridin-3-ylamino)-3-methoxy-N-(1′-methyl-1,4-bipiperidin-4-yl)benzamide

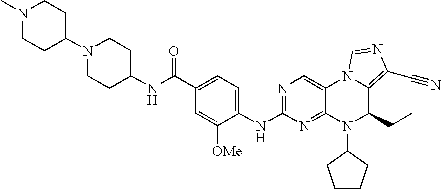

The title compound was prepared using a method analogous to Example 1 starting from (R)-4-(7-cyano-6-ethyl-5-cyclopentyl-5,6-dihydroimidazo[1,5-f]pteridin-3-ylamino)-3-methoxybenzoic and 1′-methyl-1,4′-bipiperidin-4-amine. The title compound was purified by reverse phase chromatography. ¹H NMR (400 MHz, CDCl₃) δ ppm 0.87 (t, J=7.45 Hz, 3H), 1.57-1.93 (m, 13H), 1.99-2.27 (m, 6H), 2.32 (s, 3H), 2.35-2.49 (m, 3H), 2.88-3.04 (m, 4H), 3.95-4.04 (m, 4H), 4.56-4.66 (m, 1H), 4.97 (dd, J=8.84, 3.54 Hz, 1H), 5.97 (d, J=8.08 Hz, 1H), 7.25 (dd, J=8.59, 1.77 Hz, 1H), 7.44 (d, J=1.77 Hz, 1H), 7.78 (s, 1H), 7.95 (s, 1H), 8.32 (s, 1H), 8.51 (d, J=8.34 Hz, 1H). MS (ES) [M+H] found 639.

### Example 26

### (R)-4-(7-Cyano-5-cyclopentyl-6-ethyl-5,6-dihydroimidazo[1,5-f]pteridin-3-ylamino)-N-(1′-(cyclopropylmethyl)-1,4′-bipiperidin-4-yl)-3-methoxybenzamide

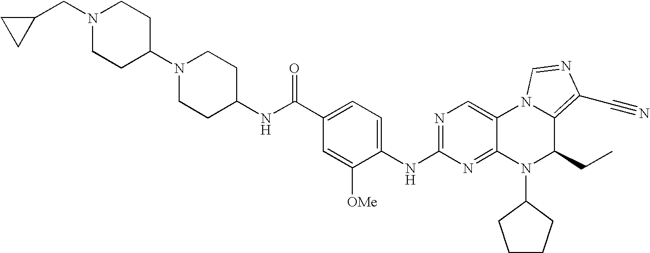

The title compound was prepared using a method analogous to Example 1 starting from (R)-4-(7-cyano-5-cyclopentyl-6-ethyl-5,6-dihydroimidazo[1,5-f]pteridin-3-ylamino)-3-methoxybenzoic acid and 1′-(cyclopropylmethyl)-1,4′-bipiperidin-4-amine hydrochloride. The title compound was purified by reverse phase chromatography to give 142 mg (65%). ¹H NMR (400 MHz, DMSO-d₆) δ ppm 0.07 (d, J=3.79 Hz, 2H) 0.40-0.52 (m, 2H) 0.74 (t, J=7.33 Hz, 3H) 0.82 (br. s., 1H) 1.35-1.65 (m, 6H) 1.66-2.07 (m, 15H) 2.11-2.34 (m, 5H) 2.88 (br. s., 2H) 3.02 (br. s., 2H) 3.74 (br. s., 1H) 3.92 (s, 3H) 4.41 (q, 1H) 5.17 (dd, J=8.21, 3.66 Hz, 1H) 7.46-7.52 (m, 2H) 8.00 (s, 1H) 8.12 (d, J=7.83 Hz, 1H) 8.24 (d, J=8.84 Hz, 1H) 8.64 (s, 1H) 8.70 (s, 1H). MS (ES) [M+H] found 679.

### Example 27

### (R)-4-(7-Cyano-5-cyclopentyl-6-ethyl-5,6-dihydroimidazo[1,5-f]pteridin-3-ylamino)-N-(1′-isobutyl-1,4′-bipiperidin-4-yl)-3-methoxybenzamide

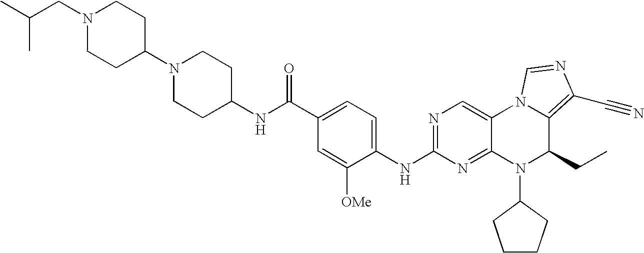

The title compound was prepared using a method analogous to Example 1 starting from ((R)-4-(7-cyano-5-cyclopentyl-6-ethyl-5,6-dihydroimidazo[1,5-f]pteridin-3-ylamino)-3-methoxybenzoic acid and 1′-isobutyl-1,4′-bipiperidin-4-amine hydrochloride with yield of 36 mg (27%). ¹H NMR (400 MHz, DMSO-d₆) δ ppm 0.74 (t, J=7.33 Hz, 3H) 0.83 (d, J=6.57 Hz, 6H) 1.42 (d, J=8.59 Hz, 2H) 1.47-1.66 (m, 5H) 1.66-1.85 (m, 10H) 1.98 (d, J=7.33 Hz, 6H) 2.18 (t, J=10.99 Hz, 3H) 2.87 (m, 4H) 3.73 (m, 1H) 3.92 (s, 3H) 4.36-4.46 (m, 1H) 5.17 (dd, J=8.21, 3.66 Hz, 1H) 7.39-7.56 (m, 2H) 8.00 (s, 1H) 8.10 (d, J=7.83 Hz, 1H) 8.24 (d, J=9.09 Hz, 1H) 8.64 (s, 1H) 8.70 (s, 1H). MS (ES) [M+H] found 681.

### Example 28

### (R)-4-(7-Cyano-5-cyclopentyl-6-ethyl-5,6-dihydroimidazo[1,5-f]pteridin-3-ylamino)-N-(1′-(cyclopropylmethyl)-1,4′-bipiperidin-4-yl)-2-fluoro-5-methoxybenzamide

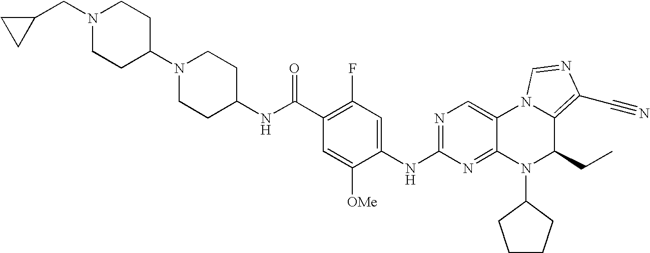

The title compound was prepared using a method analogous to Example 1 starting from (R)-4-(7-cyano-5-cyclopentyl-6-ethyl-5,6-dihydroimidazo[1,5-f]pteridin-3-ylamino)-2-fluoro-5-methoxybenzoic acid and 1′-(cyclopropylmethyl)-1,4′-bipiperidin-4-amine hydrochloride. The title compound was purified by reverse phase chromatography to give 92 mg (37%). ¹H NMR (400 MHz, DMSO-d₆) δ ppm 0.01-0.09 (m, 2H) 0.36-0.48 (m, 2H) 0.74 (t, J=7.45 Hz, 3H) 0.79-0.86 (m, 1H) 1.34-1.55 (m, 3H) 1.56-1.98 (m, 12H) 1.98-2.28 (m, 5H) 2.83 (d, 2H) 2.97 (d, 2H) 3.72 (br. s., 1H) 3.91 (s, 3H) 4.47 (q, 1H) 5.20 (dd, J=8.08, 3.79 Hz, 1H) 7.19 (d, J=6.57 Hz, 1H) 7.84-7.91 (m, 1H) 8.04 (s, 1H) 8.24 (d, J=13.39 Hz, 1H) 8.65 (s, 1H) 8.75 (s, 1H). MS (ES) [M+H] found 697.

### Example 29

### (R)-4-(7-Cyano-5-cyclopentyl-6-ethyl-5,6-dihydroimidazo[1,5-f]pteridin-3-ylamino)-2-fluoro-N-(1′-isobutyl-1,4′-bipiperidin-4-yl)-5-methoxybenzamide

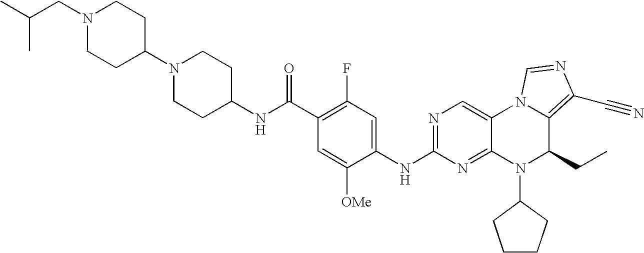

The title compound was prepared using a method analogous to Example 1 starting from (R)-4-(7-cyano-5-cyclopentyl-6-ethyl-5,6-dihydroimidazo[1,5-f]pteridin-3-ylamino)-2-fluoro-5-methoxybenzoic acid and 1′-isobutyl-1,4′-bipiperidin-4-amine hydrochloride with yield of 60 mg (43%). ¹H NMR (400 MHz, DMSO-d₆) δ ppm 0.74 (t, J=7.33 Hz, 3H) 0.83 (d, J=6.57 Hz, 6H) 1.31-1.58 (m, 5H) 1.58-1.87 (m, 12H) 1.87-2.03 (m, 5H) 2.07 (br. s., 1H) 2.10-2.32 (m, 3H) 2.83 (d, J=7.33 Hz, 4H) 3.71 (d, J=7.33 Hz, 1H) 3.91 (s, 3H) 4.39-4.53 (m, 1H) 5.20 (dd, J=8.08, 3.54 Hz, 1H) 7.19 (d, J=6.82 Hz, 1H) 7.86 (dd, J=7.71, 3.41 Hz, 1H) 8.03 (s, 1H) 8.24 (d, J=13.39 Hz, 1H) 8.65 (s, 1H) 8.75 (s, 1H). MS (ES) [M+H] found 699.

### Example 30

### (R)-4-(7-Cyano-6-ethyl-5-isopropyl-5,6-dihydroimidazo[1,5-f]pteridin-3-ylamino)-2-fluoro-5-methoxy-N-(4-((4-methylpiperazin-1-yl)methyl)phenyl)benzamide

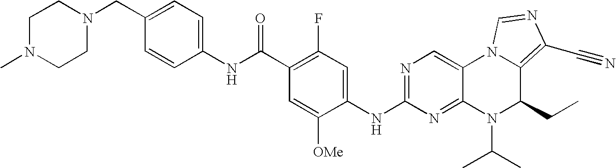

In a dry flask, (R)-4-(7-cyano-6-ethyl-5-isopropyl-5,6-dihydroimidazo[1,5-f]pteridin-3-ylamino)-2-fluoro-5-methoxybenzoic acid (90.2 mg, 0.2 mmol), 4-((4-methylpiperazin-1-yl)methyl)aniline hydrochloride (66.7 mg, 0.24 mmol) were dissolved in dry N,N-dimethylformamide (3.0 mL). Then diisopropylethylamine (107 μL, 6.0 mmol) and finally HATU (114 mg, 0.3 mmol) were added to the reaction mixture. The reaction was allowed to be continued at room temperature for 2 hours. After completion, the crude product was purified using reverse phase chromatography to give 80 mg (63% yield). ¹H NMR (400 MHz, DMSO-d₆) δ ppm 0.74 (t, J=7.33 Hz, 3H) 1.28-1.54 (m, 6H) 1.69-1.91 (m, 2H) 2.14 (s, 3H) 2.21-2.45 (m, 8H) 3.40 (s, 2H) 3.95 (s, 3H) 4.47-4.74 (m, 1H) 5.26 (dd, J=6.69, 4.67 Hz, 1H) 7.13-7.35 (m, 3H) 7.66 (d, J=8.34 Hz, 2H) 8.10 (s, 1H) 8.37 (d, J=13.14 Hz, 1H) 8.65 (s, 1H) 8.77 (s, 1H) 10.11 (s, 1H). MS (ES) [M+H] found 639.

### Example 31

### (R)-4-(7-Cyano-6-ethyl-5-isopropyl-5,6-dihydroimidazo[1,5-f]pteridin-3-ylamino)-2-fluoro-5-methoxy-N-(3-((4-methylpiperazin-1-yl)methyl)phenyl)benzamide

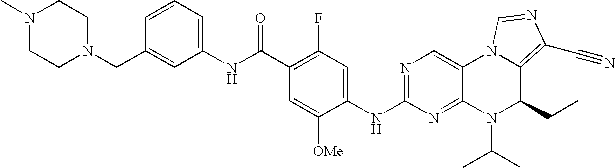

The title compound was prepared using a method analogous to Example 30 starting from (R)-4-(7-cyano-6-ethyl-5-isopropyl-5,6-dihydroimidazo[1,5-f]pteridin-3-ylamino)-2-fluoro-5-methoxybenzoic acid and 3-((4-methylpiperazin-1-yl)methyl)aniline hydrochloride with yield of 54 mg (43%). ¹H NMR (400 MHz, DMSO-d₆) δ ppm 0.74 (t, J=7.33 Hz, 3H) 1.38-1.55 (m, 6H) 1.65-1.90 (m, 2H) 2.15 (s, 3H) 2.21-2.48 (m, 8H) 3.43 (s, 2H) 3.95 (s, 3H) 4.64 (quin, J=6.82 Hz, 1H) 5.27 (dd, J=6.69, 4.67 Hz, 1H) 7.02 (d, J=7.58 Hz, 1H) 7.19-7.36 (m, 2H) 7.53-7.76 (m, 2H) 8.11 (s, 1H) 8.37 (d, J=13.14 Hz, 1H) 8.66 (s, 1H) 8.77 (s, 1H) 10.13 (s, 1H). MS (ES) [M+H] found 639.

### Example 33

### (R)-4-(7-Cyano-5-cyclopentyl-6-ethyl-5,6-dihydroimidazo[1,5-f]pteridin-3-ylamino)-N-(1′-ethyl-1,4′-bipiperidin-4-yl)-2-fluoro-5-methoxybenzamide

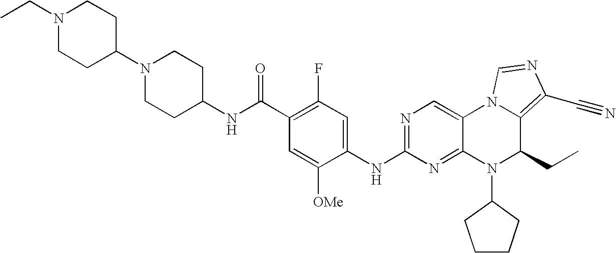

The title compound was prepared using a method analogous to Example 1 starting from (R)-4-(7-cyano-5-cyclopentyl-6-ethyl-5,6-dihydroimidazo[1,5-f]pteridin-3-ylamino)-2-fluoro-5-methoxybenzoic acid and 1′-ethyl-1,4′-bipiperidin-4-amine hydrochloride. The title compound was purified by reverse phase chromatography to give 122 mg (57%). ¹H NMR (400 MHz, DMSO-d₆) δ ppm 0.74 (t, J=7.45 Hz, 3H) 0.98 (t, J=7.07 Hz, 3H) 1.38-1.98 (m, 19H) 2.07 (br. s., 1H) 2.13-2.39 (m, 5H) 2.85 (d, J=10.86 Hz, 4H) 3.72 (d, J=5.56 Hz, 1H) 3.91 (s, 3H) 4.41-4.54 (m, 1H) 5.20 (dd, J=8.21, 3.66 Hz, 1H) 7.19 (d, J=6.57 Hz, 1H) 7.87 (dd, J=7.58, 3.54 Hz, 1H) 8.03 (s, 1H) 8.24 (d, J=13.14 Hz, 1H) 8.65 (s, 1H) 8.75 (s, 1H). MS (ES) [M+H] found 671.

### Example 34

### (R)-4-(7-Cyano-6-ethyl-5-isopropyl-5,6-dihydroimidazo[1,5-f]pteridin-3-ylamino)-3-methoxy-N-(4-((4-methylpiperazin-1-yl)methyl)phenyl)benzamide

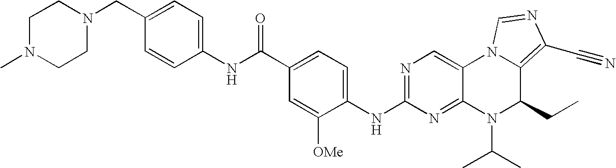

The title compound was prepared using a method analogous to Example 30 starting from (R)-4-(7-cyano-6-ethyl-5-isopropyl-5,6-dihydroimidazo[1,5-f]pteridin-3-ylamino)-3-methoxybenzoic acid and 4-((4-methylpiperazin-1-yl)methyl)aniline hydrochloride with yield of 61 mg (49%). ¹H NMR (400 MHz, DMSO-d₆) δ ppm 0.74 (t, J=7.20 Hz, 3H) 1.31-1.55 (m, 6H) 1.67-1.90 (m, 2H) 2.14 (s, 3H) 2.33 (br. s., 8H) 3.41 (s, 2H) 3.98 (s, 3H) 4.56-4.70 (m, 1H) 5.14-5.30 (m, 1H) 7.25 (d, J=8.34 Hz, 2H) 7.52-7.81 (m, 4H) 8.04 (s, 1H) 8.42 (d, J=8.34 Hz, 1H) 8.65 (s, 1H) 8.73 (s, 1H) 10.09 (s, 1H). MS (ES) [M+H] found 621.

### Example 36

### (R)-4-(7-Cyano-5-cyclopentyl-6-ethyl-5,6-dihydroimidazo[1,5-f]pteridin-3-ylamino)-2-fluoro-5-methoxy-N-(4-((4-methylpiperazin-1-yl)methyl)phenyl)benzamide

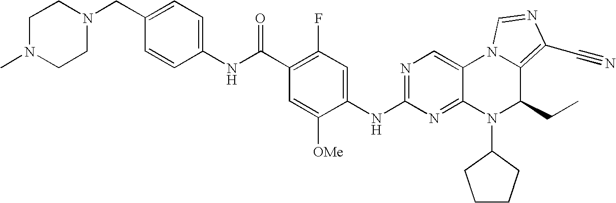

The title compound was prepared using a method analogous to Example 30 starting from (R)-4-(7-cyano-5-cyclopentyl-6-ethyl-5,6-dihydroimidazo[1,5-f]pteridin-3-ylamino)-2-fluoro-5-methoxybenzoic acid and 4-((4-methylpiperazin-1-yl)methyl)aniline hydrochloride with yield of 72 mg (54%). ¹H NMR (400 MHz, DMSO-d₆) δ ppm 0.76 (t, 3H) 1.63 (d, J=4.80 Hz, 2H) 1.67-1.88 (m, 4H) 1.88-2.03 (m, 3H) 2.08 (br. s., 1H) 2.14 (s, 3H) 2.21-2.45 (m, 8H) 3.41 (s, 2H) 3.95 (s, 3H) 4.44-4.55 (m, 1H) 5.22 (dd, J=8.21, 3.66 Hz, 1H) 7.22-7.31 (m, 3H) 7.66 (d, J=8.34 Hz, 2H) 8.10 (s, 1H) 8.31 (d, J=13.14 Hz, 1H) 8.66 (s, 1H) 8.77 (s, 1H) 10.11-10.13 (m, 1H). MS (ES) [M+H] found 665.

### Example 37

### (R)-4-(7-Cyano-6-ethyl-5-isopropyl-5,6-dihydroimidazo[1,5-f]pteridin-3-ylamino)-N-(1-(1-(cyclopropylmethyl)piperidin-4-yl)azetidin-3-yl)-3-methoxybenzamide

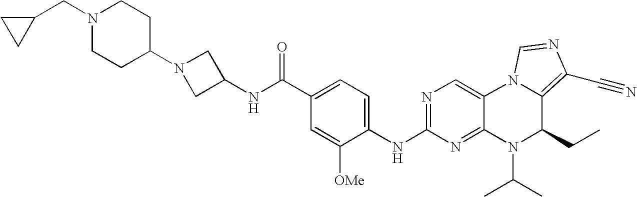

The title compound was prepared using a method analogous to Example 1 starting from (R)-4-(7-cyano-6-ethyl-5-isopropyl-5,6-dihydroimidazo[1,5-f]pteridin-3-ylamino)-3-methoxybenzoic acid and 1-(1-(cyclopropylmethyl)piperidin-4-yl)azetidin-3-amine hydrochloride with yield of 46 mg (16%). The title compound was purified by reverse phase chromatography. ¹H NMR (400 MHz, DMSO-d₆) δ ppm −0.04-0.08 (m, 2H) 0.36-0.47 (m, 2H) 0.72 (t, J=7.45 Hz, 3H) 0.76-0.84 (m, 1H) 1.17 (br. s., 2H) 1.41 (dd, 6H) 1.60 (br. s., 2H) 1.78 (t, J=7.45 Hz, 2H) 1.93 (br. s., 3H) 2.13 (d, J=6.57 Hz, 2H) 2.83 (d, J=11.12 Hz, 2H) 2.95 (t, J=7.07 Hz, 2H) 3.51 (t, J=7.33 Hz, 2H) 3.93 (s, 3H) 4.38-4.48 (m, 1H) 4.55-4.68 (m, 1H) 5.23 (dd, J=6.95, 4.67 Hz, 1H) 7.50 (d, J=1.77 Hz, 1H) 7.53 (dd, J=8.46, 1.89 Hz, 1H) 7.99 (s, 1H) 8.34 (d, J=8.34 Hz, 1H) 8.61 (d, J=6.82 Hz, 1H) 8.63 (s, 1H) 8.71 (s, 1H). MS (ES) [M+H] found 625.

### Example 38

### (R)-4-(7-Cyano-6-ethyl-5-isopropyl-5,6-dihydroimidazo[1,5-f]pteridin-3-ylamino)-N-(1-(1-(cyclopropylmethyl)piperidin-4-yl)azetidin-3-yl)-2-fluoro-5-methoxybenzamide

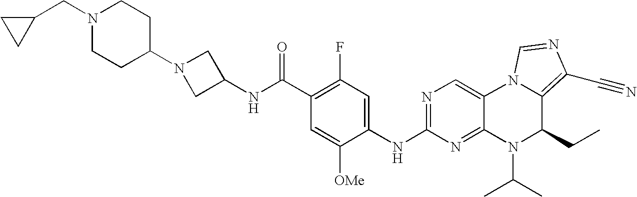

The title compound was prepared using a method analogous to Example 1 starting (R)-4-(7-cyano-6-ethyl-5-isopropyl-5,6-dihydroimidazo[1,5-f]pteridin-3-ylamino)-2-fluoro-5-methoxybenzoic acid and 1-(1-(cyclopropylmethyl)piperidin-4-yl)azetidin-3-amine hydrochloride with yield of 48 mg (17%). The title compound was purified by reverse phase chromatography. ¹H NMR (400 MHz, DMSO-d₆) δ ppm −0.02-0.10 (m, 2H) 0.38-0.49 (m, 2H) 0.73 (t, J=7.33 Hz, 3H) 0.77-0.84 (m, 1H) 1.06-1.26 (m, 3H) 1.43 (dd, 6H) 1.59 (br. s., 2H) 1.79 (t, J=7.33 Hz, 2H) 1.93 (br. s., 3H) 2.13 (d, J=6.57 Hz, 2H) 2.81 (br. s., 2H) 2.91 (t, J=7.33 Hz, 2H) 3.51 (t, J=7.20 Hz, 2H) 3.91 (s, 3H) 4.34-4.48 (m, 1H) 4.55-4.66 (m, 1H) 5.26 (dd, J=6.44, 4.93 Hz, 1H) 7.20 (d, J=6.57 Hz, 1H) 8.06 (d, J=1.01 Hz, 1H) 8.31 (d, J=13.39 Hz, 1H) 8.41 (dd, J=7.07, 3.03 Hz, 1H) 8.65 (s, 1H) 8.75 (s, 1H). MS (ES) [M+H] found 643.

### Example 39

### (R)-4-(7-Cyano-6-ethyl-5-isopropyl-5,6-dihydroimidazo[1,5-f]pteridin-3-ylamino)-N-(1-(piperidin-4-yl)azetidin-3-yl)-3-methoxybenzamide

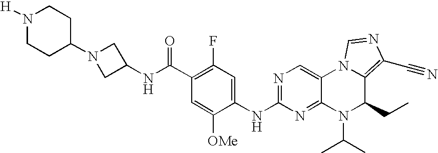

The title compound was prepared using a method analogous to Example 1 starting from (R)-4-(5-cyclopentyl-7-cyano-6-ethyl-5,6-dihydroimidazo[1,5-f]pteridin-3-ylamino)-2-fluoro-5-methoxybenzoic acid and tert-butyl 4-(3-aminoazetidin-1-yl)piperidine-1-carboxylate. The reaction was poured into water and a fine tan solid filtered and dried. This solid was then treated with TFA:methylenechloride (1:1) for 30 minutes, the solvent removed and the title compound purified by reverse phase chromatography. ¹H NMR (400 MHz, DMSO-d₆) δ ppm 0.74 (t, J=7.45 Hz, 3H) 0.93-1.06 (m, 2H) 1.44 (dd, 6H) 1.53-1.60 (m, 2H) 1.77-1.85 (m, 2H) 2.00-2.10 (m, 1H) 2.36-2.45 (m, 2H) 2.91 (t, J=7.45 Hz, 4H) 3.52 (t, J=7.45 Hz, 2H) 3.92 (s, 3H) 4.39-4.48 (m, 1H) 4.58-4.65 (m, 1H) 5.23-5.30 (m, 1H) 7.21 (d, J=6.82 Hz, 1H) 8.07 (s, 1H) 8.32 (d, J=13.39 Hz, 1H) 8.39-8.43 (m, 1H) 8.66 (s, 1H) 8.77 (s, 1H). MS (ES) [M+H] found 589.

### Example 40

### 4-(R)-7-cyano-6-ethyl-5-isopropyl-5,6-dihydroimidazo[1,5-f]pteridin-3-ylamino)-N-((1r,4R)-4-(4-(cyclopropylmethyl)piperazin-1-yl)cyclohexyl)-2-fluoro-5-methoxybenzamide

4-Amino-N-((1r,4r)-4-(4-(cyclopropylmethyl)piperazin-1-yl)cyclohexyl)-2-fluoro-5-methoxybenzamide (969 mg, 2.4 mmol) and (R)-3-chloro-6-ethyl-5-isopropyl-5,6-dihydroimidazo[1,5-f]pteridine-7-carbonitrile (604 mg, 2.0 mmol) were dissolved in 10 mL of 1:1 dioxane:dimethylacetamide then xanthophos (231.5 mg, 0.4 mmol), Cs₂CO₃ (2.60 g, 8.0 mmol) and Pd(OAc)₂ (45 mg, 0.2 mmol) were added. The mixture was microwave irradiated at 160° C. for 20 min, then cooled to room temperature and diluted with water and filtered through Celite®. The Celite® layer was then washed with EtOAc. The two layers were separated and aqueous layer neutralized with dil.HCl to pH-7 and extracted several times with EtOAc. The combined EtOAc layers were dried over Na₂SO₄ and evaporated to yield crude solid product, that were purified using preparative HPLC (eluted with a gradient of water and acetonitrile (0.05% TFA) to yield the title compound (865 mg). ¹H NMR (400 MHz, DMSO-d₆) δ ppm −0.08-0.10 (m, 2H) 0.28-0.51 (m, 2H) 0.62-0.88 (m, 4H) 1.22-1.37 (m, 4H) 1.44 (d, J=19.71 Hz, 3H) 1.42 (d, J=19.96 Hz, 3H) 1.70-1.98 (m, 7H) 2.12 (d, J=6.32 Hz, 4H) 2.28-2.45 (m, 6H) 3.68 (br. s., 1H) 3.91 (s, 3H) 4.56-4.66 (m, 1H) 5.26 (dd, J=6.69, 4.67 Hz, 1H) 7.19 (d, J=6.82 Hz, 1H) 7.83 (dd, J=7.83, 3.54 Hz, 1H) 8.04 (s, 1H) 8.30 (d, J=13.39 Hz, 1H) 8.64 (s, 1H) 8.75 (s, 1H). MS (ES) [M+H] found 671.

### Example 41

### 4-(R)-7-cyano-6-ethyl-5-isopropyl-5,6-dihydroimidazo[1,5-f]pteridin-3-ylamino)-N-((1r,4R)-4-(4-(cyclopropylmethyl)piperazin-1-yl)cyclohexyl)-2-fluoro-5-methoxybenzamide

(R)-3-Chloro-6-ethyl-5-isopropyl-5,6-dihydroimidazo[1,5-f]pteridine-7-carbonitrile (7.05 g, 23.3 mmol) and 4-amino-N-((1r,4r)-4-(4-(cyclopropylmethyl)piperazin-1-yl)cyclohexyl)-2-fluoro-5-methoxybenzamide (9.9 g, 24.5 mmol) were dissolved in isopropanol (200 mL) and conc. HCl (3.95 mL, 47.8 mmol) was added. The reaction mixture was slowly heated to 95° C. and then heating was continued for 2 days to give a suspension. The precipitated product was collected by filtration at room temperature, washed with small amount of isopropanol (20 mL) and diethyl ether (20 mL), and dried to yield the title compound as its hydrochloride salt (8.8 g). The method was repeated four times, the combined HCl salts were taken into isopropanol (300 mL) and heated at 80° C. and filtered hot, cooled to give a solid. The collected solid was then washed with diethyl ether and dried. The solid HCl salt (38.8 g) was dissolved in methanol (1000 mL) and cooled in ice bath. Solid NaHCO₃ (28.8 g) was slowly added. After 20 minutes the mixture was allowed to warm and stir at room temperature for 1 hour. The solution was then filtered through filter paper and then the volume of the solvent was reduced using rotavapor to about 600 mL before water was added (450 mL) and NaHCO3 (sat. 100 mL). The aqueous mixture was extracted with EtOAc (2×500 mL), the organic layer was dried over Na₂SO₄ and evaporated to give a solid. The solid was dissolved in a minimal amount of hot EtOH, the sides of the flask scratched and left overnight to give a solid (9.9 g, Example 41, portion 1). The mother liquor was treated with activated charcoal for 10 min., filtered through Celite®, the plug washed with EtOH, and then the solvent reduced to about 100 mL before heating to 60° C., seeded with portion 1. After about 18 hours, the mixture was filtered and dried to yield a light tan solid (Example 41, portion 2). The mother liquor was concentrated and purified by flash chromatography 100 g NH2 column, elute with dichloromethane. The purified fractions were concentrated, and the residue recrystallized from hot EtOH to give 2.88 g of a solid (Example 41, portion 3). Portion 1 (9.9 g from above), was dissolved in a minimal amount of hot EtOH and left to cool. A small amount of solid formed which was filtered off and the filtrate was combined with portion 2 and portion 3 along with enough MeOH to form a solution. This solution was treated with activated charcoal and stirred for 10 minutes, filtered through Celite®, and the plug washed with EtOH, the filtration was evaporated in vacuo to near dryness and EtOH added (125 mL), heated to 60° C., seeded with portion 1 and left overnight to give a solid. The solid was filtered, washed with EtOH and diethyl ether, and dried for 4 days in a drying oven under vacuum at 45° C. to give the title compound (15.6 g). ¹H NMR (400 MHz, DMSO-d₆) δ ppm 0.02-0.08 (m, 2H) 0.41-0.47 (m, 2H) 0.73 (t, J=7.33 Hz, 3H) 0.77-0.83 (m, 1H) 1.31 (q, 4H) 1.43 (dd, J=19.96, 6.82 Hz, 6H) 1.76-1.86 (m, 4H) 1.86-1.94 (m, 2H) 2.13 (d, J=6.57 Hz, 2H) 2.15-2.22 (m, 1H) 2.38 (br. s., 3H) 3.70 (br. s., 1H) 3.91 (s, 3H) 4.55-4.67 (m, 1H) 5.26 (dd, J=6.69, 4.67 Hz, 1H) 7.19 (d, J=6.82 Hz, 1H) 7.83 (dd, J=7.83, 3.79 Hz, 1H) 8.04 (s, 1H) 8.31 (d, J=13.39 Hz, 1H) 8.65 (s, 1H) 8.75 (s, 1H). MS (ES) [M+H] found 671, m.p: 250-254° C.

### Example 42

### 4-((R)-7-cyano-6-ethyl-5-isopropyl-5,6-dihydroimidazo[1,5-f]pteridin-3-ylamino)-N-((1r,4R)-4-(4-(cyclopropylmethyl)piperazin-1-yl)cyclohexyl)-2-fluoro-5-methoxybenzamide

The free base was prepared using a method analogous to Example 41 and 8.7 g was recrystallized from hot EtOH. The solid obtained from the EtOH was then dissolved in EtOH (150 mL) and MeOH (300 mL), treated with activated charcoal and stirred for 10 minutes. The solution was then filtered through Celite®, and the plug washed with EtOH. The solvent was then reduced about 50 mL, heated to 60° C., seeded and left overnight to give a solid. The solid was filtered, washed with EtOH and diethyl ether, and dried in a drying oven under vacuum at 45° C. for 4 days to yield 5.1 g of the title compound. ¹H NMR (400 MHz, DMSO-d₆) δ ppm 0.01-0.09 (m, 2H) 0.39-0.48 (m, 2H) 0.74 (t, J=7.45 Hz, 3H) 0.77-0.85 (m, 1H) 1.31 (q, 4H) 1.43 (dd, J=19.96, 6.82 Hz, 6H) 1.73-1.87 (m, 4H) 1.87-1.97 (m, 2H) 2.13 (d, J=6.32 Hz, 2H) 2.15-2.24 (m, 1H) 2.33 (br. s., 3H) 3.70 (br. s., 1H) 3.91 (s, 3H) 4.56-4.66 (m, 1H) 5.26 (dd, J=6.82, 4.55 Hz, 1H) 7.19 (d, J=6.82 Hz, 1H) 7.84 (dd, J=7.71, 3.66 Hz, 1H) 8.04 (s, 1H) 8.31 (d, J=13.14 Hz, 1H) 8.65 (s, 1H) 8.76 (s, 1H). MS (ES) [M+H] found 671.

### Example 43

### 4-(R)-7-Cyano-6-ethyl-5-isopropyl-5,6-dihydroimidazo[1,5-f]pteridin-3-ylamino)-N-((1r,4R)-4-(4-(cyclopropylmethyl)piperazin-1-yl)cyclohexyl)-2-fluoro-5-methoxybenzamide methanesulfonic acid salt

4-(R)-7-Cyano-6-ethyl-5-isopropyl-5,6-dihydroimidazo[1,5-f]pteridin-3-ylamino)-N-((1r,4R)-4-(4-(cyclopropylmethyl)piperazin-1-yl)cyclohexyl)-2-fluoro-5-methoxybenzamide (64.8 mg, 0.097 mmol) was dissolved in MeOH (8 mL) and methanesulfonic acid was added (9.2 mg, 0.097 mmol). The solvent was removed in vacuum and acetonitrile/water was added (3 mL, 1:1). The solution was frozen and lyophilized to yield the product as a white solid. ¹H NMR (400 MHz, DMSO-d₆) δ ppm 0.07-0.12 (m, 1H) 0.32-0.37 (m, 1H) 0.48 (d, 1H) 0.58-0.67 (m, 1H) 0.72 (t, 3H) 1.27-1.38 (m, 3H) 1.42 (dd, J=19.96, 6.82 Hz, 6H) 1.79 (br. s., 3H) 1.88-2.15 (m, 3H) 2.22-2.26 (m, 1H) 2.28 (s, 3H) 2.32 (br. s., 1H) 2.88-3.13 (m, 5H) 3.38-3.54 (m, 2H) 3.72 (br. s., 1H) 3.90 (s, 3H) 4.54-4.64 (m, 1H) 5.22-5.29 (m, 1H) 7.17 (d, J=6.57 Hz, 1H) 7.81-7.87 (m, 1H) 8.05 (s, 1H) 8.30 (d, J=13.39 Hz, 1H) 8.64 (s, 1H) 8.74 (s, 1H). MS (ES) [M+H] found 671.

### Example 44

### 4-(R)-7-Cyano-6-ethyl-5-isopropyl-5,6-dihydroimidazo[1,5-f]pteridin-3-ylamino)-N-((1r,4R)-4-(4-(cyclopropylmethyl)piperazin-1-yl)cyclohexyl)-2-fluoro-5-methoxybenzamide tri-hydrochloride salt

4-(R)-7-Cyano-6-ethyl-5-isopropyl-5,6-dihydroimidazo[1,5-f]pteridin-3-ylamino)-N-((1r,4R)-4-(4-(cyclopropylmethyl)piperazin-1-yl)cyclohexyl)-2-fluoro-5-methoxybenzamide (460 mg, 0.687 mmol) and HCl (4.0M in 1,4-dioxane) (514 μL, 2.56 mmol) was mixed in MeOH (20 mL) to give a yellow solution. After 20 minutes, the solvent was evaporated and then the resulting cream solid was dried in vacuo. (Yield: 500.8 mg, 94%). ¹H NMR (400 MHz, DMSO-d₆) δ ppm 0.07-0.12 (m, 1H) 0.32-0.37 (m, 1H) 0.48 (d, 1H) 0.58-0.67 (m, 1H) 0.72 (t, 3 H) 1.27-1.38 (m, 3H) 1.42 (dd, J=19.96, 6.82 Hz, 6H) 1.79 (br. s., 3H) 1.88-2.15 (m, 3H) 2.22-2.26 (m, 1H) 2.28 (s, 3H) 2.32 (br. s., 1H) 2.88-3.13 (m, 5H) 3.38-3.54 (m, 2H) 3.72 (br. s., 1H) 3.90 (s, 3H) 4.54-4.64 (m, 1H) 5.22-5.29 (m, 1H) 7.17 (d, J=6.57 Hz, 1H) 7.81-7.87 (m, 1H) 8.05 (s, 1H) 8.30 (d, J=13.39 Hz, 1H) 8.64 (s, 1H) 8.74 (s, 1H). MS (ES) [M+H] found 671.

### Example 45

### 4-(R)-7-cyano-6-ethyl-5-isopropyl-5,6-dihydroimidazo[1,5-f]pteridin-3-ylamino)-N-((1r,4R)-4-(4-(cyclopropylmethyl)piperazin-1-yl)cyclohexyl)-2-fluorobenzamide

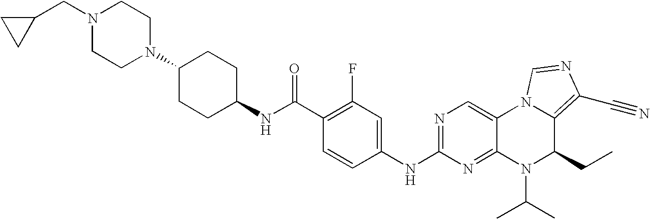

The title compound was prepared using a method analogous to Example 11 starting from (R)-4-(7-cyano-6-ethyl-5-isopropyl-5,6-dihydroimidazo[1,5-f]pteridin-3-ylamino)-2-fluorobenzoic acid and (1r,4r)-4-(4-(cyclopropylmethyl)piperazin-1-yl)cyclohexanamine. ¹H NMR (400 MHz, DMSO-d₆) δ ppm 0.15 (s, 2H) 0.44 (d, J=7.58 Hz, 2H) 0.67-0.91 (m, 6H) 1.24 (d, J=2.27 Hz, 6H) 1.37-1.53 (m, 6H) 1.80 (d, J=7.58 Hz, 7H) 2.13 (br. s., 6H) 3.56-3.77 (m, 1H) 4.53-4.78 (m, 1H) 5.24 (dd, J=6.95, 4.42 Hz, 1H) 7.37-7.66 (m, 2H) 7.88 (dd, J=14.27, 1.64 Hz, 2H) 8.64 (s, 1H) 8.74 (s, 1H) 9.93 (s, 1H). [M+H] found 641. m.p: 219-222° C.

### Example 46

### 4-(R)-7-cyano-5-cyclopentyl-6-ethyl-5,6-dihydroimidazo[1,5-f]pteridin-3-ylamino)-N-((1r,4R)-4-(4-(cyclopropylmethyl)piperazin-1-yl)cyclohexyl)-2-fluorobenzamide

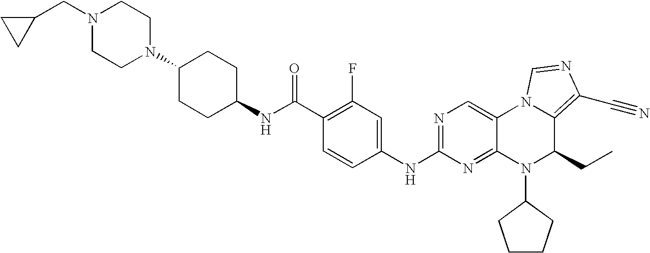

The title compound was prepared using a method analogous to Example 5 starting from (R)-4-(7-cyano-6-ethyl-5-isopropyl-5,6-dihydroimidazo[1,5-f]pteridin-3-ylamino)-2-fluorobenzoic acid and (1r,4r)-4-(4-(cyclopropylmethyl)piperazin-1-yl)cyclohexanamine. ¹H NMR (400 MHz, DMSO-d₆) δ ppm 0.27-0.42 (m, 1H) 0.48 (d, J=7.58 Hz, 1H) 0.64 (d, J=5.81 Hz, 1H) 0.70-0.90 (m, 6H) 1.21-1.42 (m, 6H) 1.45-2.17 (m, 16H) 2.84-3.16 (m, 4H) 3.40-3.52 (m, 2H) 3.69 (br. s., 1H) 4.48-4.63 (m, 1H) 5.19 (dd, J=8.72, 3.66 Hz, 1H) 7.40-7.47 (m, 1H) 7.47-7.57 (m, 1H) 7.76-8.02 (m, 2H) 8.64 (s, 1H) 8.74 (s, 1H) 9.89 (br. s., 1H). MS (ES) [M+H] found 667.

The compounds of the invention can be administered alone or in the form of a pharmaceutical composition. In practice, the compounds of the invention are usually administered in the form of pharmaceutical compositions, that is, in admixture with pharmaceutically acceptable excipients the proportion and nature of which are determined by the properties of the selected compound of the invention, the chosen route of administration, and standard pharmaceutical practice.

In another embodiment, the present invention provides pharmaceutical compositions comprising: a compound of invention and a pharmaceutically acceptable excipient.

In effecting treatment of a patient in need of such treatment, a compound of the invention can be administered in any form and route which makes the compound bioavailable. The compounds of the invention can be administered by a variety of routes, including oral and parenteral routes, more particularly by inhalation, subcutaneously, intramuscularly, intravenously, transdermally, intranasally, rectally, vaginally, occularly, topically, sublingually, and buccally, intraperitoneally, intravenously, intraarterially, transdermally, sublingually, intramuscularly, rectally, transbuccally, intranasally, intraadiposally, intrathecally and via local delivery for example by catheter or stent.

One skilled in the art can readily select the proper form and route of administration depending upon the particular characteristics of the compound selected, the disorder or condition to be treated, the stage of the disorder or condition, and other relevant circumstances. The pharmaceutical compositions of the invention may be administered to the patient, for example, in the form of tablets, capsules, cachets, papers, lozenges, wafers, elixirs, ointments, transdermal patches, aerosols, inhalants, suppositories, solutions, and suspensions.

The pharmaceutical compositions of the present invention are prepared in a manner well known in the pharmaceutical art and include at least one of the compounds of the invention as the active ingredient. The amount of a compound of the present invention may be varied depending upon its particular form and may conveniently be between 1% to about 70% of the weight of the unit dosage form. The term “pharmaceutically acceptable excipient” refers to those typically used in preparing pharmaceutical compositions and should be pharmaceutically pure and non-toxic in the amounts used. They generally are a solid, semi-solid, or liquid material which can serve as a vehicle or medium for the active ingredient. Some examples of pharmaceutically acceptable excipients are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

The present pharmaceutical compositions are preferably formulated in a unit dosage form, each dosage typically containing from about 0.5 mg to about 200 mg of the compounds of the invention. The term “unit dosage form” refers to a physically discrete unit suitable as single dosages, each unit containing a predetermined quantity of active ingredient, in association with a suitable pharmaceutical excipient, by which one or more is used throughout the dosing regime to produce the desired therapeutic effect.

In one particular variation, the composition is a pharmaceutical composition adapted for oral administration, such as a liquid formulation, for example, a solution or suspension, adapted for oral administration or a tablet or a capsule. In still another particular variation, the pharmaceutical composition is a liquid formulation adapted for parenteral administration.

In another embodiment, the invention provides a method of inhibiting a PLK: comprising, contacting the kinase with a compound of the invention. In another embodiment, the invention provides a method of inhibiting a PLK: comprising, administering a compound of the invention to a patient in order to inhibit the kinase in vivo. In a further embodiment, the invention provides a method of inhibiting a PLK: comprising, administering a first compound to a subject that is converted in vivo to a compound of the invention.

In another embodiment, compounds of the invention, including the compound of formula I, are provided for use as a medicament. The invention also provides the use of compounds of the invention for the manufacture of a medicament to treat the conditions associated with PLK described herein. The present invention also provides methods of treating conditions associated with PLK, comprising: administering to a patient in need thereof an effective amount of a compound of the invention.

As used herein terms “condition,” “disorder,” and “disease” relate to any unhealthy or abnormal state. The term “conditions associated with PLK” includes disorders and diseases such as cancer; inflammatory conditions; autoimmune diseases; cardiovascular diseases; infectious diseases; nephrological diseases; neurodegenerative diseases; skin diseases; bone diseases; the protection of proliferating cells; and other conditions. More specifically, a condition associated with PLK is selected from the group consisting of cancer of the breast, ovary, cervix, prostate, testis, esophagus, larynx, stomach, lung, including non-small cell lung cancers, bone, colon, rectum, small intestine, pancreas, thyroid, bladder, liver, kidney, pharynx, tongue, lip, mouth, brain, blood, including leukemias, and skin, including melanomas; psoriasis, alopecia, including chemotherapy agent-induced alopecia and mucositis, multiple sclerosis, colitis, and arthritis; wound healing; cardiovascular diseases, including arterioscleroses, stenoses, restenoses, and hypertrophy; viral, bacterial, fungal and/or parasitic infectious diseases, for example, cytomegalic infections, herpes, hepatitis B and C, Karposi's sarcoma, HIV diseases; nephrological diseases, for example, glomerulonephritis; chronic and acute neurodegenerative diseases, for example, Huntington's disease, amyotrophic lateral sclerosis, Parkinson's disease, AIDS dementia, Alzheimer's disease, ischemias of the brain and neurotraumas; psoriasis; bone diseases; the protection of proliferating cells, for example, hair, intestinal, blood and progenitor cells from DNA damage caused by radiation, UV treatment and/or cytostatic treatment. Where general terms are used herein to describe conditions associated with PLK it is understood that the more specifically described conditions mentioned in the various diagnostic manuals and other materials are included within the scope of this invention. For example, it is understood that the treatment of inflammatory conditions includes the treatment of arthritis and that arthritis is presently categorized into several more specific disorders, all of which are contemplated by the invention.

The terms “treat,” “treatment,” and “treating” include improvement of the conditions described herein. Also, it is also recognized that one skilled in the art may affect the conditions by treating a patient presently afflicted with the disorders or by prophylactically treating a patient believed to be susceptible to such conditions with an effective amount of a compound of invention. Thus, the terms “treat,” “treatment,” and “treating” include all processes providing slowing, interrupting, arresting, controlling, or stopping of the progression of the conditions described herein, but does not necessarily indicate a total elimination of all symptoms or a cure of the condition, and is intended to include prophylactic and therapeutic treatment of such disorders.

As used herein the term “patient” includes humans and non-human animals, for example, mammals, such as mice, rats, guinea pigs, dogs, cats, rabbits, cows, horses, sheep, goats, and pigs. The term also includes birds, fish, reptiles, amphibians, and the like. It is understood that a more particular patient is a human. Also, more particular patients are non-human mammals, such as mice, rats, and dogs.

As used herein, the term “effective amount” refers to the amount of compound of the invention which treats, upon single or multiple dose administration, a patient suffering from the mentioned condition. An effective amount can be readily determined by the attending diagnostician, as one skilled in the art, by the use of known techniques and by observing results obtained under analogous circumstances. In determining the effective amount, the dose, a number of factors are considered by the attending diagnostician, including, but not limited to: the species of patient; its size, age, and general health; the specific condition, disorder, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances. An effective amount of the present use invention, including a compound of the invention, is expected to vary from about 0.1 milligram per kilogram of body weight per day (mg/kg/day) to about 20 mg/kg/day. Specific amounts can be determined by the skilled person.

In a particular embodiment the present invention provides a method for treating cancer, comprising: administering to a patient in need thereof an effective amount of a compound of invention. In a more particular embodiment the present invention provides a method for treating therapy-resistant cancers comprising: administering to a patient in need thereof an effective amount of a compound of the invention.

A wide variety of therapeutic agents may have a therapeutic additive or synergistic effect with PLK inhibitors according to the present invention. Combination therapies that comprise one or more compounds of the present invention with one or more other therapeutic agents can be used, for example, to enhance the therapeutic effect(s) of the one or more compounds of the present invention and/or the one or more other therapeutic agents; reduce the side effects exhibited by the one or more compounds of the present invention and/or the one or more other therapeutic agents; and/or reduce the effective dose of the one or more compounds of the present invention and/or the one or more other therapeutic agents. For example, such other therapeutic agents may additively or synergistically combine with the kinase inhibitors to inhibit undesirable cell growth, such as inappropriate cell growth resulting in undesirable benign conditions or tumor growth.

Examples of such agents include anticancer agents, alkylating agents (e.g. chlorambucil, cyclophosphamide, ifosfamide, mechlorethamine, melphalan, uracil mustard, thiotepa, busulfan, carmustine, lomustine, streptozocin, altretamine, dacarbazine, procarbazine, carboplastin and cisplatin), antibiotic agents (e.g. doxorubicin, daunorubicin, epirubicin, idarubicin and anthracenedione, mitomycin C, bleomycin, dactinomycin, plicatomycin), antimetabolic agents (e.g., fluorouracil, floxuridine, methotrexate, leucovorin, hydroxyurea, thioguanine, mercaptopurine, cytarabine, pentostatin, fludarabine phosphate, cladribine, asparaginase, and gemcitabine), hormonal agents (e.g. tamoxifen, toremifene, fluoxymesterol and raloxifene), bicalutamide, nilutamide, flutamide, aminoglutethimide, anastrozole and tetrazole, ketoconazole, goserelin acetate, leuprolide, megestrol acetate and mifepristone), plant-derived agents (e.g., vincristine, vinblastine, vindesine, vinzolidine and vinorelbine, etoposide and teniposide, paclitaxel and docetaxel), and biologic agents (e.g., immuno-modulating proteins such as cytokines, monoclonal antibodies against tumor antigens, tumor suppressor genes, and cancer vaccines) and radiation therapy and radiotherapy.

The invention also provides an article of manufacture: comprising at least one compound of the invention and a label. The label may include information about the manufacturer, doses, conditions to be treated, and the use of the compound or pharmaceutical composition.

In another embodiment the invention provides a kit: comprising, at least one compound of the invention, a label, and apparatus for administration. The apparatus may include mixing vials, liquids for forming solutions or suspensions, tubing, syringes, and the like.

The activity of compounds as PLK inhibitors may be determined by a variety of methods, including in vitro, in vivo, and ex vivo methods. Example A is an in vitro enzymatic activity assay for activity against PLK1.

### Example A

Purified PLK1 may be obtained as follows. cDNA encoding human PLK1 SEQ ID No. 1, Accession Number: NM_{—}005030 was isolated by polymerase chain reaction PCR with primers SEQ ID Nos. 2 and 3 and cloned into pcDNA4/His-Max-TOPO Invitrogen, USA according to the manufacturer's manual. PCR was performed using the vector as a template. In the PCR, primers containing sequences encoding a FLAG-tag DYKDDDDK in the amino-terminal region SEQ ID No. 4 and vector sequence SEQ ID No. 5 were used. After XbaI and XhoI digestion of the PCR product, the fragment was subcloned into pFASTBAC1 Invitrogen, USA. Recombinant baculoviruses were prepared according to the procedure of the Bac-to-Bac baculovirus expression system Invitrogen, USA. Sf21 cells were purchased from Invitrogen and grown in Sf-900 II SFM medium containing 10% fetal bovine serum, 50 gentamicin and 0.1% pluronic F-68 Invitrogen, USA at 28° C. For preparation of PLK1 enzyme, Sf21 cells were infected with recombinant baculoviruses and cultured at 28° C. for 72 h. Cells were lysed and FLAG-tagged PLK1 protein SEQ ID No. 6 was purified by affinity chromatography using anti-FLAG M2 affinity gel Sigma, USA.

It should be noted that a variety of other expression systems and hosts are also suitable for the expression of PLK1, as would be readily appreciated by one of skill in the art.

The inhibition of PLK1 by the compounds was determined with the following assay that measures the phosphorylation of alpha casein by recombinant PLK1. Kinase reactions were performed at room temperature for 40 min in the kinase reaction buffer 25 mmol/L HEPES, pH 7.5, 10 mmol/L magnesium acetate, 1 mmol/L dithiothreitol containing 50 ng PLK1 enzyme, 0.1 μCi [γ-³²P]ATP, 500 nmol/L ATP and 3 μg alpha casein MP Biomedicals Inc., USA in a final volume of 50 μL. The incubation was terminated by the addition of 10% trichloroacetic acid Wako, Japan. Phosphorylated proteins were filtrated in GF/C filter plates Packard, USA with a Cell harvester Packard, USA and washed out free [γ-³²P]-ATP with 250 mmol/L phosphoric acid. Then, the plates were air dried for 60 min at 45° C., followed by the addition of 20 μL of MicroScint-O Packard, USA. The radioactivity was counted by a Top-count scintillation counter Packard, USA. The IC₅₀ values for test compounds were calculated by Prism 3.02 GraphPad Software, USA.

Examples B and C provides a method of determining in vivo activity.

### Example B

The cell culture and proliferation assay may be carried out as follows: the HT29 human colorectal adenocarcinoma cell line ATCC, USA was maintained in Dulbecco's Modified Eagle's Medium Invitrogen, USA supplemented with 10% fetal bovine serum JRH, USA. The cell proliferation assay was carried out with the Cell Titer-Glo luminescent cell viability assay Promega, USA according to the manufacture's instruction after the 72 hr treatment of the cells in the presence of the compounds. The cell viability was shown as a percentage of DMSO treated cells. The IC₅₀ values for the compounds were calculated by Prism 4 GraphPad Software, USA.

### Example C

For analysis of the cell cycle distribution and the phosphorylation of histone H3, the cells were harvested and fixed with ice-cold 70% ethanol after the 48 hr treatment of the cells in the presence of the compounds. The cells were washed twice with PBS containing 2% FCS JRH, then incubated with Alexa Flour 647-conjugated anti-phospho-histone H3 antibody Cell signaling, USA and RNase Invitrogen for 30 min at room temperature. After washing twice with PBS containing 2% FCS, the cells were counterstained with propidium iodide. The cell cycle distribution and phosphorylation of histone H3 were analyzed using the FACSCalibur system BD Bioscience, San Jose, Calif., USA.

The activity of compounds as PLK inhibitors can be further assessed in vivo using BALB/cA Jcl-nu/nu mice bearing the HCT116 or the HT29 cells inoculated subcutaneously in auxiliary area. The growth retardation may be determined, for example, by caliper measurements of the tumor volume.

pIC50 values are the negative of the log of the IC₅₀s and may be calculated by non-linear curve fitting of the compound concentrations and fluorescence intensities to the standard pIC₅₀ equation. The method of Example A gave pIC₅₀ values greater than 7 for the examples above. Values for particular compounds from Example A are given in Table 1 below.

TABLE 1

| Example Number | pIC₅₀ PLK1 |
|---|---|
| 19 | 8.7 |
| 20 | 8.4 |
| 21 | 8.5 |
| 22 | 8.4 |
| 26 | 9.0 |
| 27 | 8.7 |
| 29 | 8.9 |
| 31 | 8 |
| 37 | 8.5 |
| 38 | 8.5 |

What is claimed is:

1. A compound of the formula 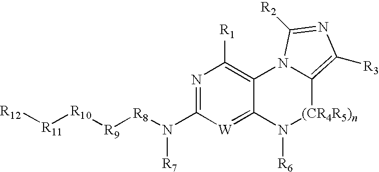
wherein
R₁ is selected from the group consisting of hydrogen, halo, amino, alkylamino, C₁₋₄ alkoxy, C₁₋₄ alkyl, and SOₓX₁
x is selected from the group consisting of 0, 1, and 2;
Xᵢ is selected from the group consisting of optionally substituted C₁₋₄ alkyl, optionally substituted C₃₋₈ cycloalkyl, and optionally substituted C₄₋₁₂ aryl;
R₂ is selected from the group consisting of hydrogen, halo, and C₁₋₄ alkyl;
R₃ is selected from the group consisting of amino, hydrogen, halo, nitro, cyano, optionally substituted C₁₋₆ alkyl, C₁₋₄ alkoxy, C₁₋₉ amide, C₁₋₅ oxycarbonyl, and N(X₂)(X₃),
X₂ is selected from the group consisting of hydrogen and C₁₋₄ alkyl and X₃ is selected from the group consisting of C₁₋₄ alkyl, C₁₋₇ alkylcarbonyl, and C₁₋₆ sulfonyl;
n is selected from the group consisting of 1 and 2;
R₄, each time taken, is independently selected from the group consisting of hydrogen, halo, hydroxy, optionally substituted C₁₋₁₀ alkyl, optionally substituted C₄₋₁₂ aryloxy, optionally substituted heteroC₁₋₁₀ aryloxy, optionally substituted C₃₋₈ cycloalkyl, optionally substituted C₄₋₁₂ aryl, and optionally substituted heteroC₁₋₁₀ aryl;
R₅, each time taken, is independently selected from the group consisting of hydrogen, halo, optionally substituted C₁₋₁₀ alkyl, optionally substituted C₃₋₈ cycloalkyl, optionally substituted C₄₋₁₂ aryl, and optionally substituted heteroC₁₋₁₀ aryl; or
R₄ and R₅ taken together with the carbon to which they are attached to form C═O; or
R₄ and R₅ taken together with the carbon to which they are attached form an optionally substituted C₃₋₈ cycloalkyl ring; or
when n is 2, one of R₄ or R₅ on different carbons is taken together along with the carbons to which they are attached to form an optionally substituted C₃₋₈ cycloalkyl ring;
R₆ is selected from the group consisting of hydrogen, optionally substituted C₁₋₆ alkyl, C₁₋₃ sulfonyl, and optionally substituted C₃₋₈ cycloalkyl;
R₇ is hydrogen or a substituent convertible in vivo to hydrogen;
R₈ is selected from the group consisting of optionally substituted C₄₋₁₂ arylene, optionally substituted heteroC₁₋₁₀ arylene, optionally substituted C₃₋₈ cycloalkylene, optionally substituted heteroC₃₋₆ cycloalkylene, optionally substituted C₇₋₁₂ bicycloalkylene, and optionally substituted heteroC₃₋₁₂ bicycloalkylene;
R₉ is selected from the group consisting of —CONJ₉-, —NJ₉CO—, —NJ₉-, —SO₂NJ₉-, —NJ₉SO₂—,
J₉ is selected from the group consisting of hydrogen and C₁₋₄ alkyl;
R₁₀ is selected from the group consisting of optionally substituted C₄₋₁₂ arylene, optionally substituted heteroC₁₋₁₀ arylene, optionally substituted C₃₋₈ cycloalkylene, optionally substituted heteroC₃₋₆ cycloalkylene, optionally substituted C₇₋₁₂ bicycloalkylene, and optionally substituted heteroC₃₋₁₂ bicycloalkylene;
R₁₁ is selected from the group consisting of optionally substituted C₁₋₄ alkylene, optionally substituted C₁₋₄ azaalkylene, optionally substituted C₃₋₈ cycloalkylene, and optionally substituted heteroC₃₋₆ cycloalkylene;
R₁₂ is optionally substituted C₃₋₈ cycloalkyl, when R₁₁ is selected from the group consisting of optionally substituted C₁₋₄ alkylene and optionally substituted C₁₋₄ azaalkylene;
R₁₂ is optionally substituted C₁₋₄ alkyl, when R₁₁ is selected from the group consisting of optionally substituted C₃₋₈ cycloalkylene and optionally substituted heteroC₃₋₆ cycloalkylene;
W is N or CR₁₃;
R₁₃ is selected from the group consisting of hydrogen, halo, nitro, cyano, optionally substituted C₁₋₆ alkyl, and optionally substituted C₁₋₄ alkoxy;
and pharmaceutically acceptable salts thereof.

2. A compound of claim 1 wherein R₈ is optionally substituted 1,4-phenylene counting from the point of attachment to the nitrogen bearing R₈ being the 1-position wherein one substituent is 3-fluoro or 3-chloro.

3. A compound of claim 1 wherein R₈ is optionally substituted 1,4-phenylene counting from the point of attachment to the nitrogen bearing R₈ being the 1-position wherein one substituent is 6-methoxy.

4. A compound of any one of claims 1 to 3 wherein R₂ is hydrogen.

5. A compound of any one of claims 1 to 4 wherein R₃ is selected from the group consisting of hydrogen, halo, cyano, trifluoromethyl, hydroxy substituted C₁₋₄ alkyl, C₁₋₉ amide, and C₁₋₅ oxycarbonyl.

6. A compound of anyone of claim 3, 4, or 5 wherein is optionally substituted C₄₋₁₂ arylene wherein one substituent is C₁₋₄ alkoxy and one substituent is halogen, R₉ is —CONH—, R₁₀ is C₃₋₈ cycloalkylene, and R₁₁ is optionally substituted heteroC₃₋₆ cycloalkylenecycloalkylene.

7. A compound of any one of claims 1 to 6 wherein one of R₄ or R₅ are not hydrogen, (C₁₋₁₀)alkyl, or (C₃₋₁₂)cycloalkyl.

8. The compound of claim 1 which is 4-(7-cyano-6-ethyl-5-isopropyl-5,6-dihydroimidazo[1,5-f]pteridine-3-ylamino)-N-(4-(4-cyclopropylmethyl)piperazin-1-yl)cyclohexyl-2-fluoro-5-methoxybenzamide.

9. The compound of claim 1 which is 4-(R)-7-Cyano-6-ethyl-5-isopropyl-5,6-dihydroimidazo[1,5-f]pteridin-3-ylamino)-N-((1r,4R)-4-(4-(cyclopropylmethyl)piperazin-1-yl)cyclohexyl)-2-fluoro-5-methoxybenzamide.

10. A pharmaceutical composition, comprising: a compound of any one of claims 1 to 8 and a pharmaceutically acceptable excipient.

11. The use of a compound of any one of claims 1 to 9 as a medicament.

12. The use of a compound of any one of claims 1 to 9 for the manufacture of a medicament to treat the conditions associated with PLK.

13. A method of treating conditions associated with PLK, comprising: administering to a patient in need thereof an effective amount of a compound of any one of claims 1 to 9.


SEQUENCE LISTING (6 sequences)

SEQ ID NO: 1 - DNA, 1809 bp, Homo sapien
atgagtgctgcagtgactgcagggaagctggcacgggcaccggccgaccctgggaaagccggggtccccggagttgcagctcccggagctccggcggcggctccaccggcgaaagagatcccggaggtcctagtggacccacgcagccggcggcgctatgtgcggggccgctttttgggcaagggcggctttgccaagtgcttcgagatctcggacgcggacaccaaggaggtgttcgcgggcaagattgtgcctaagtctctgctgctcaagccgcaccagagggagaagatgtccatggaaatatccattcaccgcagcctcgcccaccagcacgtcgtaggattccacggctttttcgaggacaacgacttcgtgttcgtggtgttggagctctgccgccggaggtctctcctggagctgcacaagaggaggaaagccctgactgagcctgaggcccgatactacctacggcaaattgtgcttggctgccagtacctgcaccgaaaccgagttattcatcgagacctcaagctgggcaaccttttcctgaatgaagatctggaggtgaaaataggggattttggactggcaaccaaagtcgaatatgacggggagaggaagaagaccctgtgtgggactcctaattacatagctcccgaggtgctgagcaagaaagggcacagtttcgaggtggatgtgtggtccattgggtgtatcatgtataccttgttagtgggcaaaccaccttttgagacttcttgcctaaaagagacctacctccggatcaagaagaatgaatacagtattcccaagcacatcaaccccgtggccgcctccctcatccagaagatgcttcagacagatcccactgcccgcccaaccattaacgagctgcttaatgacgagttctttacttctggctatatccctgcccgtctccccatcacctgcctgaccattccaccaaggttttcgattgctcccagcagcctggaccccagcaaccggaagcccctcacagtcctcaataaaggcttggagaaccccctgcctgagcgtccccgggaaaaagaagaaccagtggttcgagagacaggtgaggtggtcgactgccacctcagtgacatgctgcagcagctgcacagtgtcaatgcctccaagccctcggagcgtgggctggtcaggcaagaggaggctgaggatcctgcctgcatccccatcttctgggtcagcaagtgggtggactattcggacaagtacggccttgggtatcagctctgtgataacagcgtgggggtgctcttcaatgactcaacacgcctcatcctctacaatgatggtgacagcctgcagtacatagagcgtgacggcactgagtcctacctcaccgtgagttcccatcccaactccttgatgaagaagatcaccctccttaaatatttccgcaattacatgagcgagcacttgctgaaggcaggtgccaacatcacgccgcgcgaaggtgatgagctcgcccggctgccctacctacggacctggttccgcacccgcagcgccatcatcctgcacctcagcaacggcagcgtgcagatcaacttcttccaggatcacaccaagctcatcttgtgcccactgatggcagccgtgacctacatcgacgagaagcgggacttccgcacataccgcctgagtctcctggaggagtacggctgctgcaaggagctggccagccggctccgctacgcccgcactatggtggacaagctgctgagctcacgctcggccagcaaccgtctcaaggcctcc

SEQ ID NO: 2 - DNA, 27 bp, Artificial Sequence
  DNA sequence encoding PCR Primers
agtgctgcagtgactgcagggaagctg

SEQ ID NO: 3 - DNA, 29 bp, Artificial Sequence
  DNA sequence encoding PCR primers
ttaggaggccttgagacggttgctggccg

SEQ ID NO: 4 - DNA, 69 bp, Artificial Sequence
  DNA sequence encoding PCR primers
aaatctagagccaccatggactacaaggacgacgatgacaagagtgctgcagtgactgcagggaagctg

SEQ ID NO: 5 - DNA, 27 bp, Artificial Sequence
  DNA sequence encoding PCR primers
tggcaactagaaggcacagtcgaggct

SEQ ID NO: 6 - protein, 1682 aa, Artificial Sequence
  Protein sequence encoding FLAG-tagged PLK1
METASPTYRLYSASPASPASPASPLYSSERALAALAVALTHRALAGLYLYSLEALAARGALAPRALAASPPRGLYLYSALAGLYVALPRGLYVALALAALAPRGLYALAPRALAALAALAPRPRALALYSGLILEPRGLVALLEVALASPPRARGSERARGARGARGTYRVALARGGLYARGPHELEGLYLYSGLYGLYPHEALALYSCYSPHEGLILESERASPALAASPTHRLYSGLVALPHEALAGLYLYSILEVALPRLYSSERLELELELYSPRHISGLNARGGLLYSMETSERMETGLILESERILEHISARGSERLEALAHISGLNHISVALVALGLYPHEHISGLYPHEPHEGLASPASNASPPHEVALPHEVALVALLEGLLECYSARGARGARGSERLELEGLLEHISLYSARGARGLYSALALETHRGLPRGLALAARGTYRTYRLEARGGLNILEVALLEGLYCYSGLNTYRLEHISARGASNARGVALILEHISARGASPLELYSLEGLYASNLEPHELEASNGLASPLEGLVALLYSILEGLYASPPHEGLYLEALATHRLYSVALGLTYRASPGLYGLARGLYSLYSTHRLECYSGLYTHRPRASNTYRILEALAPRGLVALLESERLYSLYSGLYHISSERPHEGLVALASPVALTRPSERILEGLYCYSILEMETTYRTHRLELEVALGLYLYSPRPRPHEGLTHRSERCYSLELYSGLTHRTYRLEARGILELYSLYSASNGLTYRSERILEPRLYSHISILEASNPRVALALAALASERLEILEGLNLYSMETLEGLNTHRASPPRTHRALAARGPRTHRILEASNGLLELEASNASPGLPHEPHETHRSERGLYTYRILEPRALAARGLEPRILETHRCYSLETHRILEPRPRARGPHESERILEALAPRSERSERLEASPPRSERASNARGLYSPRLETHRVALLEASNLYSGLYLEGLASNPRLEPRGLARGPRARGGLLYSGLGLPRVALVALARGGLTHRGLYGLVALVALASPCYSHISLESERASPMETLEGLNGLNLEHISSERVALASNALASERLYSPRSERGLARGGLYLEVALARGGLNGLGLALAGLASPPRALACYSILEPRILEPHETRPVALSERLYSTRPVALASPTYRSERASPLYSTYRGLYLEGLYTYRGLNLECYSASPASNSERVALGLYVALLEPHEASNASPSERTHRARGLEILELETYRASNASPGLYASPSERLEGLNTYRILEGLARGASPGLYTHRGLSERTYRLETHRVALSERSERHISPRASNSERLEMETLYSLYSILETHRLELELYSTYRPHEARGASNTYRMETSERGLHISLELELYSALAGLYALAASNILETHRPRARGGLGLYASPGLLEALAARGLEPRTYRLEARGTHRTRPPHEARGTHRARGSERALAILEILELEHISLESERASNGLYSERVALGLNILEASNPHEPHEGLNASPHISTHRLYSLEILELECYSPRLEMETALAALAVALTHRTYRILEASPGLLYSARGASPPHEARGTHRTYRARGLESERLELEGLGLTYRGLYCYSCYSLYSGLLEALASERARGLEARGTYRALAARGTHRMETVALASPLYSLELESERSERARGSERALASERASNARGLELYSALASER